(19) United States
(12) Patent Application Publication
(10) Pub. No.: US 2014/0228366 A1
(43) Pub. Date: Aug. 14, 2014

(54) HETEROARYL-SUBSTITUTED PYRAZOLOPYRIDINES AND USE THEREOF AS SOLUBLE GUANYLATE CYCLASE STIMULATORS

(71) Applicants: Markus Follmann, Koln (DE); Johannes-Peter Stasch, Solingen (DE); Gorden Redlich, Bochum (DE); Nils Griebenow, Dormagen (DE); Dieter Lang, Velbert (DE); Frank Wunder, Wuppertal (DE)

(72) Inventors: Markus Follmann, Koln (DE); Johannes-Peter Stasch, Solingen (DE); Gorden Redlich, Bochum (DE); Nils Griebenow, Dormagen (DE); Dieter Lang, Velbert (DE); Frank Wunder, Wuppertal (DE)

(73) Assignee: BAYER INTELLECTUAL PROPERTY GMBH, Monheim (DE)

(21) Appl. No.: 14/131,017

(22) PCT Filed: Jul. 5, 2012
(86) PCT No.: PCT/EP2012/063155

§ 371 Date: Feb. 27, 2014

(30) Foreign Application Priority Data
Jul. 6, 2011 (DE) 102011078715.1
Jan. 11, 2012 (DE) 102012200354.1

(51) Int. Cl. C07D 471/04; A61K 31/519; A61K 31/53

(52) U.S. Cl. CPC C07D 471/04; A61K 31/53; A61K 31/519
USPC 514/243; 544/184; 544/280; 514/265.1

(57) ABSTRACT

The present application relates to novel heteroaryl-substituted pyrazolopyridines, to processes for their preparation, to their use alone or in combinations for the treatment and/or prophylaxis of diseases, and to their use for producing medicaments for the treatment and/or prophylaxis of diseases, in particular for the treatment and/or prophylaxis of cardiovascular disorders.

The present application relates to novel heteroaryl-substituted pyrazolopyridines, to processes for their preparation, to their use alone or in combinations for the treatment and/or prophylaxis of diseases, and to their use for producing medicaments for the treatment and/or prophylaxis of diseases, in particular for the treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family known to date can be divided into two groups either according to structural features or according to the type of ligands: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory site. This is of central importance for the activation mechanism NO can bind to the iron atom of heme and thus markedly increase the activity of the enzyme. Heme-free preparations cannot, by contrast, be stimulated by NO. Carbon monoxide (CO) is also able to bind to the central iron atom of heme, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the abovementioned processes. Under pathophysiological conditions, the NO/cGMP system can be suppressed, which can lead, for example, to hypertension, platelet activation, increased cell proliferation, endothelial dysfunction, atherosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Hitherto, for the therapeutic stimulation of the soluble guanylate cyclase use has exclusively been made of compounds such as organic nitrates whose effect is based on NO. The latter is formed by bioconversion and activates soluble guanylate cyclase by attack at the central iron atom of heme. In addition to the side effects, the development of tolerance is one of the decisive disadvantages of this type of treatment.

In recent years, some substances have been described which stimulate soluble guanylate cyclase directly, i.e. without prior release of NO, such as, for example, 3-(5′-hydroxymethyl-2′-furyl)-1-benzylindazole [YC-1; Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

WO 00/06569 discloses fused pyrazole derivatives and WO 03/095451 carbamate-substituted 3-pyrimidinylpyrazolopyridines as stimulators of soluble guanylate cyclase. WO 2010/065275 and WO 2011/149921 disclose substituted pyrrolo- and dihydropyridopyrimidines as sGC activators. 3-Furylindazoles having heteroaryl substituents in the 1-position as sGC stimulators are described in Straub A. et al., Bioorg. Med. Chem. Lett. 11 (2001), 781-784 and WO 98/16507.

It was an object of the present invention to provide novel substances which act as stimulators of soluble guanylate cyclase and which have an identical or improved therapeutic profile compared to compounds known from the prior art, for example with respect to their in vivo properties such as their pharmacokinetic and pharmacodynamic behaviour and/or their metabolic profile and/or their dose-activity relationship.

The present invention provides compounds of the general formula (I)

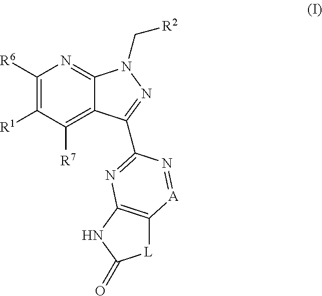

in which
A represents nitrogen or CR³,
   where
   R³ represents hydrogen, deuterium, halogen, difluoromethyl, trifluoromethyl, (C₁-C₄)-alkyl, (C₂-C₄)-alkenyl, (C₂-C₄)-alkynyl, cyclopropyl, cyclobutyl, hydroxy, amino, phenyl or 5- or 6-membered heteroaryl,
      in which (C₁-C₄)-alkyl, (C₂-C₄)-alkenyl, (C₂-C₄)-alkynyl, phenyl and 5- or 6-membered heteroaryl may be substituted by 1 to 3 substituents independently of one another selected from the group consisting of fluorine, difluoromethyl, trifluoromethyl, (C₁-C₄)-alkyl, difluoromethoxy, trifluoromethoxy, (C₁-C₄)-alkoxy, (C₁-C₄)-alkoxycarbonyl, cyclopropyl and cyclobutyl,
         L represents a *—CR^{4A}R^{4B}—(CR^{5A}R^{5B})ₚ-# group,
   where
   * represents the point of attachment to the carbonyl group,
   # represents the point of attachment to the pyrimidine ring or triazine ring,
   p represents a number 0, 1 or 2,
   R^{4A} represents hydrogen, fluorine, (C₁-C₄)-alkyl, hydroxy or amino,
      in which (C₁-C₄)-alkyl may be substituted by 1 to 3 substituents independently of one another selected from the group consisting of fluorine, trifluoromethyl, hydroxy, hydroxycarbonyl, (C₁-C₄)-alkoxycarbonyl and amino,
   R^{4B} represents hydrogen, fluorine, difluoromethyl, trifluoromethyl, (C₁-C₆)-alkyl, (C₁-C₄)-alkoxycarbonylamino, cyano, (C₃-C₇)-cycloalkyl, difluoromethoxy, trifluoromethoxy, phenyl or a group of the formula -M-R⁸,
      in which (C₁-C₆)-alkyl may be substituted by 1 to 3 substituents independently of one another selected from the group consisting of fluorine, cyano, trifluoromethyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl and amino,
      and in which
      M represents a bond or (C₁-C₄)-alkanediyl,
      R⁸ represents —(C═O)ᵣ—OR⁹, —(C═O)ᵣ—NR⁹R¹⁰, —C(═S)—NR⁹R¹⁰, —NR⁹—(C═O)—R¹², —NR⁹—(C═O)—NR¹⁰R¹¹, —NR⁹—SO₂—NR¹⁰R¹¹, —NR⁹—SO₂—R¹², —S(O)ₛ—R¹², —SO₂—NR⁹R¹⁰, 4- to 7-membered heterocyclyl, phenyl or 5- or 6-membered heteroaryl,
      in which
      r represents the number 0 or 1,
      s represents the number 0, 1 or 2,
      R⁹, R¹⁰ and R¹¹ independently of one another each represent hydrogen, (C₁-C₆)-alkyl, (C₃-C₈)-cycloalkyl, 4- to 7-membered heterocyclyl, phenyl or 5- or 6-membered heteroaryl,
      or
      R⁹ and R¹⁰ together with the atom(s) to which they are respectively attached form a 4- to 7-membered heterocycle,
         in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkylamino and di-(C₁-C₆)-alkylamino,
      or
      R¹⁰ and R¹¹ together with the atom(s) to which they are respectively attached form a 4- to 7-membered heterocycle,
         in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkylamino and di-(C₁-C₆)-alkylamino,
      R¹² represents (C₁-C₆)-alkyl or (C₃-C₇)-cycloalkyl,
      or
      R⁹ and R¹² together with the atom(s) to which they are respectively attached form a 4- to 7-membered heterocycle,
         in which the 4- to 7-membered heterocycle for its part may be substituted by 1 or 2 substituents independently of one another selected from the group consisting of cyano, trifluoromethyl, (C₁-C₆)-alkyl, hydroxy, oxo, alkoxy, trifluoromethoxy, (C₁-C₆)-alkoxycarbonyl, amino, mono-(C₁-C₆)-alkylamino and di-(C₁-C₆)-alkylamino,
      and
      in which 4- to 7-membered heterocyclyl, phenyl and 5- or 6-membered heteroaryl for their part may be substituted by 1 to 3 substituents independently of one another selected from the group consisting of halogen, cyano, difluoromethyl, trifluoromethyl, (C₁-C₆)-alkyl, (C₃-C₇)-cycloalkyl, hydroxy, oxo, thioxo and (C₁-C₄)-alkoxy,
      and
      in which the aforementioned (C₁-C₄)-alkyl, (C₁-C₆)-alkyl, (C₃-C₈)-cycloalkyl and 4- to 7-membered heterocyclyl groups, unless stated otherwise, may each independently of one another additionally be substituted by 1 to 3 substituents independently of one another selected from the group consisting of fluorine, difluoromethyl, trifluoromethyl, (C₁-C₆)-alkyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl, amino, phenyl, 4- to 7-membered heterocyclyl and 5- or 6-membered heteroaryl,
   or
   R^{4A} and R^{4B} together with the carbon atom to which they are attached form a (C₂-C₄)-alkenyl group, an oxo group, a 3- to 6-membered carbocycle or a 4- to 7-membered heterocycle,
      in which the 3- to 6-membered carbocycle and the 4- to 7-membered heterocycle may be substituted by 1 or 2 substituents independently of one another selected from the group consisting of fluorine and (C₁-C₄)-alkyl,
   R^{5A} represents hydrogen, fluorine, (C₁-C₄)-alkyl, (C₁-C₄)-alkoxycarbonyl or hydroxy,
   R^{5B} represents hydrogen, fluorine, (C₁-C₄)-alkyl or trifluoromethyl,
R¹ represents hydrogen, halogen, cyano, difluoromethyl, trifluoromethyl, (C₁-C₄)-alkyl or (C₃-C₇)-cycloalkyl,
R² represents 5- or 6-membered heteroaryl,
   where 5- and 6-membered heteroaryl may be substituted by 1 or 2 fluorine substituents,
R⁶ represents hydrogen, cyano, difluoromethyl, trifluoromethyl, (C₁-C₄)-alkyl or (C₃-C₇)-cycloalkyl,
R⁷ represents hydrogen, cyano, difluoromethyl, trifluoromethyl, (C₁-C₄)-alkyl or (C₃-C₇)-cycloalkyl,
and their N-oxides, salts, solvates, salts of the N-oxides and solvates of the N-oxides and salts.

Compounds according to the invention are the compounds of the formula (I) and the N-oxides, salts, solvates and solvates of the N-oxides and salts thereof, the compounds, encompassed by formula (I), of the formulae specified hereinafter and the N-oxides, salts, solvates and solvates of the N-oxides and salts thereof, and the compounds encompassed by formula (I) and specified hereinafter as working examples and the N-oxides, salts, solvates and solvates of the N-oxides and salts thereof, to the extent that the compounds encompassed by formula (I) and specified hereinafter are not already N-oxides, salts, solvates and solvates of the N-oxides and salts.

Preferred salts in the context of the present invention are physiologically acceptable salts of the compounds according to the invention. Also encompassed are salts which are not themselves suitable for pharmaceutical applications but can be used, for example, for isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates refer to those forms of the compounds according to the invention which, in the solid or liquid state, form a complex by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers or diastereomers and the respective mixtures thereof. The stereoisomerically uniform constituents can be isolated from such mixtures of enantiomers and/or diastereomers in a known manner; chromatography processes are preferably used for this, in particular HPLC chromatography on an achiral or chiral phase.

Where the compounds according to the invention can occur in tautomeric forms, the present invention encompasses all the tautomeric forms.

The present invention also encompasses all suitable isotopic variants of the inventive compounds. An isotopic variant of a compound according to the invention is understood here to mean a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I, and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. Furthermore, the incorporation of isotopes, for example of deuterium, can lead to particular therapeutic advantages as a consequence of greater metabolic stability of the compound, for example an extension of the half-life in the body or a reduction in the active dose required; such modifications of the compounds according to the invention may therefore, in some cases, also constitute a preferred embodiment of the present invention. Isotopic variants of the compounds according to the invention can be prepared by the processes known to those skilled in the art, for example by the methods described below and the instructions reproduced in the working examples, by using corresponding isotopic modifications of the particular reagents and/or starting compounds therein.

Moreover, the present invention also encompasses prodrugs of the compounds according to the invention. The term “prodrugs” here denotes compounds which may themselves be biologically active or inactive, but are converted (for example metabolically or hydrolytically) to inventive compounds during their residence time in the body.

In the context of the present invention, the substituents, unless specified otherwise, are each defined as follows:

Alkyl in the context of the invention is a straight-chain or branched alkyl radical having the number of carbon atoms specified in each case. The following may be mentioned by way of example and by way of preference: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl and 2-ethylbutyl.

Cycloalkyl or carbocycle in the context of the invention is a monocyclic saturated alkyl radical having the number of carbon atoms specified in each case. The following may be mentioned by way of example and by way of preference: cyclopropyl, cyclobutyl, cyclopentyl, cyclohexyl and cycloheptyl.

Alkanediyl in the context of the invention is a straight-chain or branched divalent alkyl radical having 1 to 4 carbon atoms. The following may be mentioned by way of example and by way of preference: methylene, ethane-1,2-diyl, ethane-1,1-diyl, propane-1,3-diyl, propane-1,1-diyl, propane-1,2-diyl, propane-2,2-diyl, butane-1,4-diyl, butane-1,2-diyl, butane-1,3-diyl and butane-2,3-diyl.

Alkenyl in the context of the invention is a straight-chain or branched alkenyl radical having 2 to 4 carbon atoms and a double bond. The following may be mentioned by way of example and by way of preference: vinyl, allyl, isopropenyl and n-but-2-en-1-yl.

Alkynyl in the context of the invention is a straight-chain or branched alkynyl radical having 2 to 4 carbon atoms and one triple bond. The following may be mentioned by way of example and by way of preference: ethynyl, n-prop-1-yn-1-yl, n-prop-2-yn-1-yl, n-but-2-yn-1-yl and n-but-3-yn-1-yl.

Alkoxy in the context of the invention is a straight-chain or branched alkoxy radical having 1 to 6 or 1 to 4 carbon atoms. The following may be mentioned by way of example: methoxy, ethoxy, n-propoxy, isopropoxy, 1-methylpropoxy, n-butoxy, isobutoxy, tert-butoxy, n-pentoxy, isopentoxy, 1-ethylpropoxy, 1-methylbutoxy, 2-methylbutoxy, 3-methylbutoxy and n-hexoxy. Preference is given to a straight-chain or branched alkoxy radical having 1 to 4 carbon atoms. The following may be mentioned by way of example and by way of preference: methoxy, ethoxy, n-propoxy, isopropoxy, 1-methylpropoxy, n-butoxy, isobutoxy, tert-butoxy.

Alkoxycarbonyl in the context of the invention is a straight-chain or branched alkoxy radical having 1 to 4 carbon atoms and a carbonyl group attached to the oxygen. The following may be mentioned by way of example and by way of preference: methoxycarbonyl, ethoxycarbonyl, n-propoxycarbonyl, isopropoxycarbonyl and tert-butoxycarbonyl.

Alkoxycarbonylamino in the context of the invention is an amino group having a straight-chain or branched alkoxycarbonyl substituent which has 1 to 4 carbon atoms in the alkyl chain and is attached via the carbonyl group to the nitrogen atom. The following may be mentioned by way of example and by way of preference: methoxycarbonylamino, ethoxycarbonylamino, propoxycarbonylamino, n-butoxycarbonylamino, isobutoxycarbonylamino and tert-butoxycarbonylamino.

Monoalkylamino in the context of the invention is an amino group having a straight-chain or branched alkyl substituent having 1 to 6 carbon atoms. The following may be mentioned by way of example and by way of preference: methylamino, ethylamino, n-propylamino, isopropylamino and tert-butylamino.

Dialkylamino in the context of the invention is an amino group having two identical or different, straight-chain or branched alkyl substituents each having 1 to 6 carbon atoms. The following may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Heterocyclyl or heterocycle in the context of the invention is a saturated heterocycle which has a total of 4 to 7 ring atoms and contains one or two ring heteroatoms from the group consisting of N, O, S, SO and/or SO₂. The following may be mentioned by way of example: azetidinyl, oxetanyl, pyrrolidinyl, pyrazolidinyl, imidazolinyl, tetrahydrofuranyl, piperidinyl, piperazinyl, tetrahydropyranyl, morpholinyl, thiomorpholinyl and dioxidothiomorpholinyl. Preference is given to azetidinyl, oxetanyl, pyrrolidinyl, tetrahydrofuranyl, piperidinyl, tetrahydropyranyl and morpholinyl.

5- or 6-membered heteroaryl in the context of the invention is a monocyclic aromatic heterocycle (heteroaromatic) which has a total of 5 or 6 ring atoms, contains up to three identical or different ring heteroatoms from the group consisting of N, O and/or S and is attached via a ring carbon atom or optionally via a ring nitrogen atom. The following may be mentioned by way of example and by way of preference: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl. Preference is given to: pyrazolyl, oxazolyl, thiazolyl, triazolyl, oxadiazolyl, thiadiazolyl, pyridyl and pyrimidinyl.

8- or 9-membered heteroaryl in the context of the invention is a bicyclic aromatic or partly unsaturated heterocycle which has a total of 8 or 9 ring atoms and contains at least two nitrogen atoms and up to two further, identical or different ring heteroatoms from the group of N, O and/or S. The following may be mentioned by way of example: dihydrothienopyrazolyl, thienopyrazolyl, pyrazolopyrazolyl, imidazothiazolyl, tetrahydrocyclopentapyrazolyl, dihydrocyclopentapyrazolyl, tetrahydroindazolyl, dihydroindazolyl, indazolyl, pyrazolopyridinyl, tetrahydropyrazolopyridinyl, pyrazolopyrimidinyl, imidazopyridinyl and imidazopyridazinyl.

Halogen in the context of the invention is fluorine, chlorine, bromine and iodine. Preference is given to bromine and iodine.

An oxo group in the context of the invention is an oxygen atom attached via a double bond to a carbon atom.

A thiooxo group in the context of the invention is a sulphur atom attached via a double bond to a carbon atom.

In the formula of the group which may represent L, the end point of the line marked by the symbol * or # does not represent a carbon atom or a CH₂ group, but is part of the bond to the respective atom to which L is attached.

When radicals in the compounds according to the invention are substituted, the radicals, unless specified otherwise, may be mono- or polysubstituted. In the context of the present invention, all radicals which occur more than once are defined independently of one another. Substitution by one, two or three identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” or “preclusion” are used synonymously in the context of the present invention and refer to the avoidance or reduction of the risk of contracting, experiencing, suffering from or having a disease, a condition, a disorder, an injury or a health problem, or a development or progression of such states and/or the symptoms of such states.

The treatment or prevention of a disease, a condition, a disorder, an injury or a health problem may be partial or complete.

The compounds of the formula (I-1) form a sub-group of the compounds of the formula (I) according to the invention in which R⁶ and R⁷ represent hydrogen.

In the context of the present invention, preference is given to compounds of the formula (I) in which
A represents nitrogen or CR³,
   where
   R³ represents hydrogen, deuterium, fluorine, iodine, difluoromethyl, trifluoromethyl, (C₁-C₄)-alkyl, vinyl, allyl, ethynyl, cyclopropyl, cyclobutyl, hydroxy, pyrazolyl or pyridyl,
      where (C₁-C₄)-alkyl, vinyl, allyl, ethynyl and pyridyl may be substituted by 1 or 2 substituents independently of one another selected from the group consisting of methyl, cyclopropyl and cyclobutyl,
L represents a *—CR^{4A}R^{4B}—(CR^{5A}R^{5B})^{p}—# group,
   where
   * represents the point of attachment to the carbonyl group,
   # represents the point of attachment to the pyrimidine ring or triazine ring,
   p represents a number 0, 1 or 2,
   R^{4A} represents hydrogen, fluorine, methyl, ethyl, hydroxy or amino,
   R^{4B} represents hydrogen, fluorine, difluoromethyl, trifluoromethyl, (C₁-C₄)-alkyl, methoxycarbonylamino, cyano, cyclopropyl, cyclobutyl, cyclopentyl, phenyl or a group of the formula -M-R⁸,
      in which (C₁-C₄)-alkyl may be substituted by 1 to 3 substituents independently of one another selected from the group consisting of fluorine, cyano, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino, and in which
      M represents a bond or methylene,
      R⁸ represents —(C═O)ᵣ—NR⁹R¹⁰, —C(═S)—NR⁹R¹⁰, oxadiazolonyl, oxadiazolethionyl, phenyl, oxazolyl, thiazolyl, pyrazolyl, triazolyl, oxadiazolyl, thiadiazolyl, pyridyl, pyrimidinyl or pyrazinyl, in which
      r represents the number 0 or 1,
      R⁹ and R¹⁰ independently of one another each represent hydrogen, methyl, ethyl, isopropyl, cyclopropyl, cyclobutyl, cyclopentyl, oxetanyl, azetidinyl, tetrahydrofuranyl, pyrrolidinyl, tetrahydropyranyl, piperidinyl, piperazinyl, morpholinyl, phenyl, pyrazolyl or pyridyl,
         in which methyl, ethyl and isopropyl may additionally be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
      and
      in which oxadiazolonyl, oxadiazolethionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
   or
   R^{4A} and R^{4B} together with the carbon atom to which they are attached form a cyclopropyl, cyclobutyl, cyclopentyl, azetidinyl, tetrahydrofuranyl, pyrrolidinyl or tetrahydropyranyl ring,
      in which the cyclopropyl, cyclobutyl, cyclopentyl, azetidinyl, tetrahydrofuranyl, pyrrolidinyl and tetrahydropyranyl ring may be substituted by 1 or 2 substituents independently of one another selected from the group consisting of fluorine and methyl,
   R^{5A} represents hydrogen, fluorine, methyl, ethyl or hydroxy,
   R^{5B} represents hydrogen, fluorine, methyl, ethyl or trifluoromethyl,
R¹ represents hydrogen or fluorine,
R² represents thienyl, pyridyl, pyrimidinyl, pyrazinyl or pyridazinyl,
   where thienyl, pyridyl, pyrimidinyl, pyrazinyl and pyridazinyl may be substituted by 1 or 2 fluorine substituents,
R⁶ represents hydrogen or methyl,
R⁷ represents hydrogen,
and the salts, solvates and solvates of the salts thereof.

In the context of the present invention, preference is also given to compounds of the formula (I) in which
A represents nitrogen or CR³,
   where
   R³ is hydrogen, fluorine, difluoromethyl, trifluoromethyl, methyl, ethyl, cyclopropyl or cyclobutyl,
L represents a *—CR^{4A}R^{4B}—(CR^{5A}R^{5B})ₚ—# group, where
   * represents the point of attachment to the carbonyl group,
   # represents the point of attachment to the pyrimidine ring or triazine ring,
   p represents a number 0,
   R^{4A} represents hydrogen, fluorine, methyl, ethyl, hydroxy or amino,
   R^{4B} represents hydrogen, fluorine, difluoromethyl, trifluoromethyl, methyl, ethyl, methoxycarbonylamino, cyclopropyl, cyclobutyl, cyclopentyl or a group of the formula -M-R⁸,
      in which methyl and ethyl may be substituted by 1 to 3 substituents independently of one another selected from the group consisting of fluorine, cyano, trifluoromethyl, cyclopropyl, cyclobutyl, hydroxy, difluoromethoxy, trifluoromethoxy, methoxy, ethoxy, hydroxycarbonyl, methoxycarbonyl, ethoxycarbonyl and amino,
      and in which
      M represents a bond,
      R⁸ represents —(C═O)ᵣ—NR⁹R¹⁰, phenyl, thiazolyl, triazolyl, oxadiazolyl, thiadiazolyl or pyrimidinyl,
         in which
         r represents the number 1,
         R⁹ and R¹⁰ independently of one another each represent hydrogen or cyclopropyl,
         and
         in which phenyl, thiazolyl, triazolyl, oxadiazolyl, thiadiazolyl and pyrimidinyl for their part may be substituted by 1 or 2 substituents independently of one another selected from the group consisting of fluorine, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl and cyclobutylmethyl,
   or
   R^{4A} and R^{4B} together with the carbon atom to which they are attached form a cyclopropyl, cyclobutyl, cyclopentyl, azetidinyl, tetrahydrofuranyl, pyrrolidinyl or tetrahydropyranyl ring,
      in which the cyclopropyl, cyclobutyl, cyclopentyl, azetidinyl, tetrahydrofuranyl, pyrrolidinyl and tetrahydropyranyl ring may be substituted by 1 or 2 substituents independently of one another selected from the group consisting of fluorine and methyl,
R¹ represents hydrogen or fluorine,
R² represents thienyl, pyridyl or pyrimidinyl,
   where thienyl, pyridyl and pyrimidinyl may be substituted by 1 or 2 fluorine substituents,
R⁶ represents hydrogen or methyl,
R⁷ represents hydrogen,
and the salts, solvates and solvates of the salts thereof.

In the context of the present invention, particular preference is given to compounds of the formula (I) in which
A represents nitrogen or CR³,
   where
   R³ represents hydrogen,
L represents a *—CR^{4A}R^{4B}—(CR^{5A}R^{5B})ₚ—# group,
   where
   * represents the point of attachment to the carbonyl group,
   # represents the point of attachment to the pyrimidine ring or triazine ring,
   p represents a number 0,
   R^{4A} represents hydrogen, fluorine, methyl or hydroxy,
   R^{4B} represents hydrogen, fluorine, trifluoromethyl, 2,2,2-trifluoroethyl or methyl,
R¹ represents hydrogen or fluorine,
R² represents thienyl, pyridyl or pyrimidinyl,
   where thienyl, pyridyl and pyrimidinyl may be substituted by 1 or 2 fluorine substituents,
R⁶ represents hydrogen,
R⁷ represents hydrogen,
and the salts, solvates and solvates of the salts thereof.

In the context of the present invention, preference is also given to compounds of the formula (I-1) in which

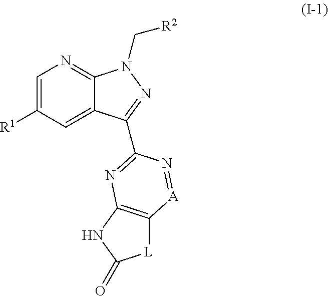

in which
A represents nitrogen or CR³,
   where
   R³ represents hydrogen, deuterium, halogen, difluoromethyl, trifluoromethyl, (C₁-C₄)-alkyl, cyclopropyl, cyclobutyl, hydroxy or amino,
L represents a *—CR^{4A}R^{4B}—(CR^{5A}R^{5B})ₚ—# group,
   where
   * represents the point of attachment to the carbonyl group,
   # represents the point of attachment to the pyrimidine or triazine ring,
   p represents a number 0, 1 or 2,
   R^{4A} represents hydrogen, fluorine, (C₁-C₄)-alkyl, hydroxy or amino,
      in which (C₁-C₄)-alkyl may be substituted by 1 to 3 substituents independently of one another selected from the group consisting of fluorine, trifluoromethyl, hydroxy, hydroxycarbonyl, (C₁-C₄)-alkoxycarbonyl and amino,
   R^{4B} represents hydrogen, fluorine, (C₁-C₄)-alkyl, trifluoromethyl, (C₁-C₄)-alkoxycarbonylamino or phenyl,
      in which (C₁-C₄)-alkyl may be substituted by 1 to 3 substituents independently of one another selected from the group consisting of fluorine, trifluoromethyl, hydroxy, hydroxycarbonyl, (C₁-C₄)-alkoxycarbonyl and amino,
   or
   R^{4A} and R^{4B} together with the carbon atom to which they are attached form an oxo group, a 3- to 6-membered carbocycle or a 4- to 6-membered heterocycle,
      in which the 3- to 6-membered carbocycle and the 4- to 6-membered heterocycle may be substituted by 1 or 2 substituents independently of one another selected from the group consisting of fluorine and (C₁-C₄)-alkyl,
   or
   R^{4A} and R^{4B} together with the carbon atom to which they are attached form a (C₂-C₄)-alkenyl group,
   R^{5A} represents hydrogen, fluorine, (C₁-C₄)-alkyl or hydroxy,
   R^{5B} represents hydrogen, fluorine, (C₁-C₄)-alkyl or trifluoromethyl,
R¹ represents hydrogen or fluorine,
R² represents 5- or 6-membered heteroaryl,
   where 5- and 6-membered heteroaryl may be substituted by 1 or 2 fluorine substituents,
and their N-oxides, salts, solvates, salts of the N-oxides and solvates of the N-oxides and salts.

In the context of the present invention, preference is given to compounds of the formula (I-1) in which
A represents nitrogen or CR³,
   where
   R³ represents hydrogen, deuterium, fluorine, iodine, difluoromethyl, trifluoromethyl, (C₁-C₄)-alkyl, cyclopropyl, cyclobutyl or hydroxy,
L represents a *—CR^{4A}R^{4B}—(CR^{5A}R^{5B})ₚ—# group,
   where
   * represents the point of attachment to the carbonyl group,
   # represents the point of attachment to the pyrimidine or triazine ring,
   p represents a number 0, 1 or 2,
   R^{4A} represents hydrogen, fluorine, methyl, ethyl or hydroxy,
   R^{4B} represents hydrogen, fluorine, methyl, ethyl or trifluoromethyl,
   or
   R^{4A} and R^{4B} together with the carbon atom to which they are attached form a cyclopropyl, cyclobutyl, cyclopentyl, azetidinyl, pyrrolidinyl, tetrahydrofuranyl, piperidinyl or tetrahydropyranyl ring,
   R^{5A} represents hydrogen, fluorine, methyl, ethyl or hydroxy,
   R^{5B} represents hydrogen, fluorine, methyl, ethyl or trifluoromethyl,
R¹ represents hydrogen or fluorine,
R² represents pyridyl, pyrimidinyl, pyrazinyl or pyridazinyl,
   where pyridyl, pyrimidinyl, pyrazinyl and pyridazinyl may be substituted by 1 or 2 fluorine substituents,
and the salts, solvates and solvates of the salts thereof.

In the context of the present invention, preference is also given to compounds of the formula (I-1) in which
A represents CR³,
   where
   R³ represents amino,
L represents a *—CR^{4A}R^{4B}—(CR^{5A}R^{5B})ₚ—# group,
   where
   * represents the point of attachment to the carbonyl group,
   # represents the point of attachment to the pyrimidine ring or triazine ring,
   p represents a number 0, 1 or 2,
   R^{4A} represents hydrogen, fluorine, methyl, ethyl or hydroxy,
   R^{4B} represents hydrogen, fluorine, methyl, ethyl or trifluoromethyl,
   or
   R^{4A} and R^{4B} together with the carbon atom to which they are attached form a cyclopropyl, cyclobutyl, cyclopentyl, azetidinyl, pyrrolidinyl, tetrahydrofuranyl, piperidinyl or tetrahydropyranyl ring,
   R^{5A} represents hydrogen, fluorine, methyl, ethyl or hydroxy,
   R^{5B} represents hydrogen, fluorine, methyl, ethyl or trifluoromethyl,
R¹ represents hydrogen or fluorine,
R² represents pyridyl, pyrimidinyl, pyrazinyl or pyridazinyl,
   where pyridyl, pyrimidinyl, pyrazinyl and pyridazinyl may be substituted by 1 or 2 fluorine substituents,
and the salts, solvates and solvates of the salts thereof.

In the context of the present invention, particular preference is given to compounds of the formula (I-1) in which
A represents CR³,
   where
   R³ represents hydrogen,
L represents a *—CR^{4A}R^{4B}—(CR^{5A}R^{5B})ₚ—# group,
   where
   * represents the point of attachment to the carbonyl group,
   # represents the point of attachment to the pyrimidine ring,
   p represents a number 0 or 1,
   R^{4A} represents hydrogen, methyl or hydroxy,
   R^{4B} represents hydrogen, fluorine, methyl or trifluoromethyl,
   or
   R^{4A} and R^{4B} together with the carbon atom to which they are attached form a cyclopropyl or cyclobutyl ring,
      in which the cyclopropyl and the cyclobutyl ring may be substituted by 1 or 2 substituents independently of one another selected from the group consisting of fluorine and methyl,
R¹ represents hydrogen or fluorine,
R² represents 3-fluoropyrid-2-yl or pyrimidin-2-yl,
and the salts, solvates and solvates of the salts thereof.

In the context of the present invention, particular preference is also given to compounds of the formula (I-1) in which
A represents nitrogen,
L represents a *—CR^{4A}R^{4B}—(CR^{5A}R^{5B})ₚ—# group,
   where
   * represents the point of attachment to the carbonyl group,
   # represents the point of attachment to the triazine ring,
   p represents a number 0 or 1,
   R^{4A} represents hydrogen, methyl or hydroxy,
   R^{4B} represents hydrogen, fluorine, methyl or trifluoromethyl,
   or
   R^{4A} and R^{4B} together with the carbon atom to which they are attached form a cyclopropyl or cyclobutyl ring,
      in which the cyclopropyl and the cyclobutyl ring may be substituted by 1 or 2 substituents independently of one another selected from the group consisting of fluorine and methyl,
R¹ represents hydrogen or fluorine,
R² represents 3-fluoropyrid-2-yl or pyrimidin-2-yl,
and the salts, solvates and solvates of the salts thereof.

In the context of the present invention, preference is also given to compounds of the formulae (I) and (I-1) in which R¹ represents H, and the salts, solvates and solvates of the salts thereof.

In the context of the present invention, preference is also given to compounds of the formulae (I) and (I-1) in which R¹ represents fluorine, and the salts, solvates and solvates of the salts thereof.

In the context of the present invention, preference is also given to compounds of the formulae (I) and (I-1) in which A represents N or CH, and the salts, solvates and solvates of the salts thereof.

In the context of the present invention, preference is also given to compounds of the formulae (I) and (I-1) in which A represents N, and the salts, solvates and solvates of the salts thereof.

In the context of the present invention, preference is also given to compounds of the formulae (I) and (I-1) in which A represents CH, and the salts, solvates and solvates of the salts thereof.

In the context of the present invention, preference is also given to compounds of the formulae (I) and (I-1) in which
A represents nitrogen or CR³,
   where
   R³ represents hydrogen, deuterium, fluorine, iodine, difluoromethyl, trifluoromethyl, (C₁-C₄)-alkyl, cyclopropyl, cyclobutyl or hydroxy,
      and the salts, solvates and solvates of the salts thereof.

In the context of the present invention, preference is also given to compounds of the formulae (I) and (I-1) in which
A represents CR³,
   where
   R³ represents hydrogen, deuterium, fluorine, iodine, difluoromethyl, trifluoromethyl, (C₁-C₄)-alkyl, cyclopropyl, cyclobutyl or hydroxy,
      and the salts, solvates and solvates of the salts thereof.

In the context of the present invention, preference is also given to compounds of the formulae (I) and (I-1) in which
A represents CR³,
   where
   R³ represents amino,
      and the salts, solvates and solvates of the salts thereof.

In the context of the present invention, preference is also given to compounds of the formulae (I) and (I-1) in which
A represents CR³,
   where
   R³ represents hydrogen,
L represents a *—CR^{4A}R^{4B}—(CR^{5A}R^{5B})ₚ—# group,
   where
   * represents the point of attachment to the carbonyl group,
   # represents the point of attachment to the pyrimidine or triazine ring,
   p represents a number 0,
   R^{4A} represents hydrogen, fluorine, methyl or hydroxy,
   R^{4B} represents hydrogen, fluorine, methyl or trifluoromethyl,
      and the salts, solvates and solvates of the salts thereof.

In the context of the present invention, preference is also given to compounds of the formulae (I) and (I-1) in which
A represents CR³,
   where
   R³ represents hydrogen,
L represents a *—CR^{4A}R^{4B}—(CR^{5A}R^{5B})ₚ—# group,
   where
   * represents the point of attachment to the carbonyl group,
   # represents the point of attachment to the pyrimidine or triazine ring,
   p represents a number 0,
   R^{4A} represents methyl,
   R^{4B} represents methyl,
      and the salts, solvates and solvates of the salts thereof.

In the context of the present invention, preference is also given to compounds of the formulae (I) and (I-1) in which
A represents nitrogen,
L represents a *—CR^{4A}R^{4B}—(CR^{5A}R^{5B})ₚ—# group,
   where
   * represents the point of attachment to the carbonyl group,
   # represents the point of attachment to the triazine ring,
   p represents a number 0,
   R^{4A} represents hydrogen, fluorine, methyl or hydroxy,
   R^{4B} represents hydrogen, fluorine, methyl or trifluoromethyl,
      and the salts, solvates and solvates of the salts thereof.

In the context of the present invention, preference is also given to compounds of the formulae (I) and (I-1) in which
A represents nitrogen,
L represents a *—CR^{4A}R^{4B}—(CR^{5A}R^{5B})ₚ—# group,
   where
   * represents the point of attachment to the carbonyl group,
   # represents the point of attachment to the triazine ring,
   p represents a number 0,
   R^{4A} represents methyl,
   R^{4B} represents methyl,
      and the salts, solvates and solvates of the salts thereof.

In the context of the present invention, preference is also given to compounds of the formulae (I) and (I-1) in which
L represents a *—CR^{4A}R^{4B}—(CR^{5A}R^{5B})ₚ—# group,
   where
   * represents the point of attachment to the carbonyl group,
   # represents the point of attachment to the pyrimidine or triazine ring,
   p represents a number 0,
   R^{4A} and R^{4B} together with the carbon atom to which they are attached form a cyclopropyl, cyclobutyl, cyclopentyl, azetidinyl, pyrrolidinyl, tetrahydrofuranyl, piperidinyl or tetrahydropyranyl ring,
      and the salts, solvates and solvates of the salts thereof.

In the context of the present invention, preference is also given to compounds of the formulae (I) and (I-1) in which
where
* represents the point of attachment to the carbonyl group,
# represents the point of attachment to the pyrimidine or triazine ring,
p represents a number 0,
R^{4A} represents hydrogen, fluorine, methyl or hydroxy,
R^{4B} represents hydrogen, fluorine, methyl or trifluoromethyl,
   and the salts, solvates and solvates of the salts thereof.

In the context of the present invention, preference is also given to compounds of the formulae (I) and (I-1) in which
L represents a *—CR^{4A}R^{4B}—(CR^{5A}R^{5B})ₚ) —# group,
   where
   * represents the point of attachment to the carbonyl group,
   # represents the point of attachment to the pyrimidine or triazine ring,
   p represents a number 0,
   R^{4A} represents methyl,
   R^{4B} represents methyl,
      and the salts, solvates and solvates of the salts thereof.

In the context of the present invention, preference is also given to compounds of the formulae (I) and (I-1) in which
L represents a *—CR^{4A}R^{4B}—(CR^{5A}R^{5B})ₚ—# group,
   where
   * represents the point of attachment to the carbonyl group,
   # represents the point of attachment to the pyrimidine or triazine ring,
   p represents a number 0,
   R^{4A} and R^{4B} together with the carbon atom to which they are attached form a cyclopropyl or cyclobutyl ring,
      in which the cyclopropyl and the cyclobutyl ring may be substituted by 1 or 2 substituents independently of one another selected from the group consisting of fluorine and methyl,
         and the salts, solvates and solvates of the salts thereof.

In the context of the present invention, preference is also given to compounds of the formulae (I) and (I-1) in which
R² represents furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl or triazinyl,
   where furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl may be substituted by 1 or 2 fluorine substituents,
      and the salts, solvates and solvates of the salts thereof.

In the context of the present invention, preference is also given to compounds of the formulae (I) and (I-1) in which
R² represents pyridyl, pyrimidinyl, pyridazinyl, pyrazinyl or triazinyl,
   where pyridyl, pyrimidinyl, pyridazinyl, and pyrazinyl may be substituted by 1 or 2 fluorine substituents,
      and the salts, solvates and solvates of the salts thereof.

In the context of the present invention, preference is also given to compounds of the formulae (I) and (I-1) in which
R² represents pyrid-2-yl, pyrid-4-yl, pyrimidin-2-yl, pyrimidin-5-yl or pyrazin-2-yl,
   where pyrid-2-yl and pyrid-4-yl may be substituted by 1 or 2 fluorine substituents, and the salts, solvates and solvates of the salts thereof.

In the context of the present invention, preference is also given to compounds of the formulae (I) and (I-1) in which
R² represents 3-fluoropyrid-2-yl or pyrimidin-2-yl,
   and the salts, solvates and solvates of the salts thereof.

In the context of the present invention, preference is also given to compounds of the formulae (I) and (I-1) in which
R² represents 3-fluoropyrid-2-yl,
   and the salts, solvates and solvates of the salts thereof.

In the context of the present invention, preference is also given to compounds of the formula (I) in which
A represents CR³,
   where
   R³ represents hydrogen,
L represents a *—CR^{4A}R^{4B}—(CR^{5A}R^{5B})ₚ—# group,
   where
   * represents the point of attachment to the carbonyl group,
   # represents the point of attachment to the pyrimidine ring,
   p represents a number 0,
   R^{4A} represents hydrogen, fluorine, methyl, ethyl, hydroxy or amino,
   R^{4B} represents a group of the formula -M-R⁸,
      in which
      M represents a bond,
      R⁸ represents —(C═O)ᵣ—NR⁹R¹⁰, —C(═S)—NR⁹R¹⁰, oxadiazolonyl, oxadiazolethionyl, phenyl, oxazolyl, thiazolyl, pyrazolyl, triazolyl, oxadiazolyl, thiadiazolyl, pyridyl, pyrimidinyl or pyrazinyl,
         in which
         r represents the number 0,
         R⁹ and R¹⁰ independently of one another each represent hydrogen, methyl, ethyl, isopropyl, cyclopropyl, cyclobutyl, cyclopentyl, oxetanyl, azetidinyl, tetrahydrofuranyl, pyrrolidinyl, tetrahydropyranyl, piperidinyl, piperazinyl, morpholinyl, phenyl, pyrazolyl or pyridyl,
            in which methyl, ethyl and isopropyl for their part may be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
         and
         in which oxadiazolonyl, oxadiazolethionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
            and the salts, solvates and solvates of the salts thereof.

In the context of the present invention, preference is also given to compounds of the formula (I) in which
A represents N,
L represents a *—CR^{4A}R^{4B}—(CR^{5A}R^{5B})ₚ—# group,
   where
   * represents the point of attachment to the carbonyl group,
   # represents the point of attachment to the triazine ring,
   p represents a number 0,
   R^{4A} represents hydrogen, fluorine, methyl, ethyl, hydroxy or amino,
   R^{4B} represents a group of the formula -M-R⁸,
      in which
      M represents a bond,
      R⁸ represents —(C═O)ᵣ—NR⁹R¹⁰, —C(═S)—NR⁹R¹⁰, oxadiazolonyl, oxadiazolethionyl, phenyl, oxazolyl, thiazolyl, pyrazolyl, triazolyl, oxadiazolyl, thiadiazolyl, pyridyl, pyrimidinyl or pyrazinyl,
         in which
         r represents the number 0,
         R⁹ and R¹⁰ independently of one another each represent hydrogen, methyl, ethyl, isopropyl, cyclopropyl, cyclobutyl, cyclopentyl, oxetanyl, azetidinyl, tetrahydrofuranyl, pyrrolidinyl, tetrahydropyranyl, piperidinyl, piperazinyl, morpholinyl, phenyl, pyrazolyl or pyridyl,
            in which methyl, ethyl and isopropyl for their part may be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
         and
         in which oxadiazolonyl, oxadiazolethionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
            and the salts, solvates and solvates of the salts thereof.

The individual radical definitions specified in the particular combinations or preferred combinations of radicals are, independently of the particular combinations of the radicals specified, also replaced as desired by radical definitions of other combinations.

Particular preference is given to combinations of two or more of the preferred ranges mentioned above.

The invention furthermore provides a process for preparing the compounds of the formula (I) according to the invention, characterized in that a compound of the formula (II)

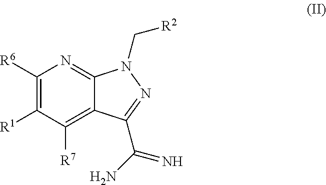

in which R¹, R², R⁶ and R⁷ each have the meanings given above
[A] is reacted in an inert solvent in the presence of a suitable base with a compound of the formula (III)

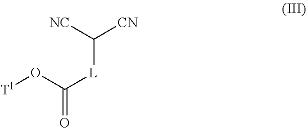

in which L has the meaning given above and
T¹ represents (C₁-C₄)-alkyl
to give a compound of the formula (IV)

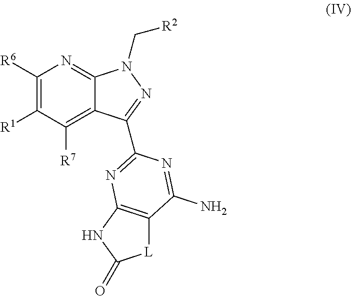

in which L, R¹, R², R⁶ and R⁷ each have the meanings given above,
this is then converted with isopentyl nitrite and a halogen equivalent into a compound of the formula (V)

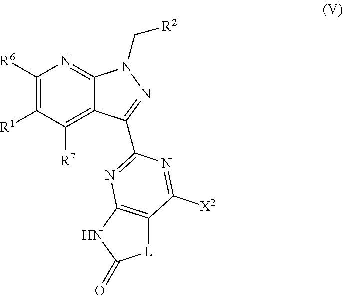

in which L, R¹, R², R⁶ and R⁷ each have the meanings given above and
X² represents bromine or iodine,
and this is then reacted in an inert solvent, in the presence of a suitable transition metal catalyst, to give a compound of the formula (I-A)

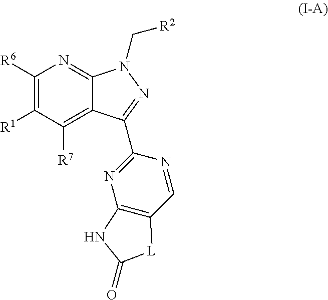

in which L, R¹, R², R⁶ and R⁷ each have the meanings given above,
or
[B] is reacted in an inert solvent in the presence of a suitable base with hydrazine hydrate to give a compound of the formula (VI)

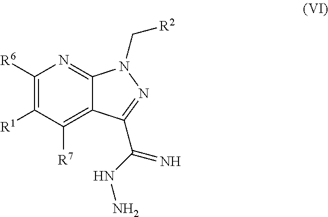

in which R¹, R², R⁶ and R⁷ each have the meanings given above, this is then reacted in an inert solvent with a compound of the formula (VII)

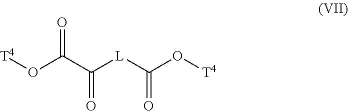

in which L has the meaning given above and
T⁴ represents (C₁-C₄)-alkyl
to give a compound of the formula (VIII)

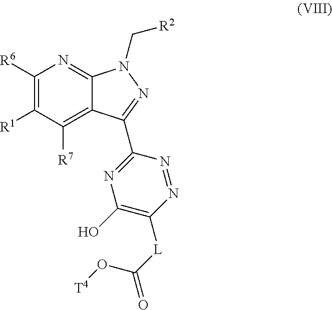

in which L, R¹, R², R⁶, R⁷ and T⁴ each have the meanings given above, this is then converted with phosphoryl chloride into a compound of the formula (IX)

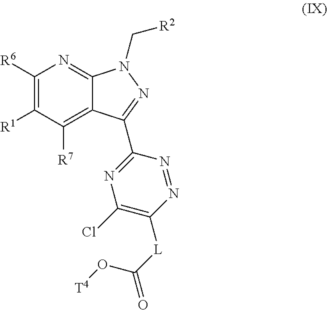

in which L, R¹, R², R⁶, R⁷ and T⁴ each have the meanings given above,
and this is reacted directly with ammonia to give a compound of the formula (X)

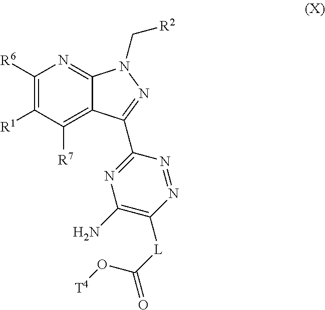

in which L, R¹, R², R⁶, R⁷ and T⁴ each have the meanings given above,
and finally cyclized in an inert solvent, optionally in the presence of a suitable base, to give a compound of the formula (I-B)

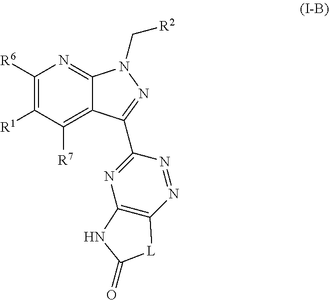

in which L, R¹, R², R⁶ and R⁷ each have the meanings given above,
   and the resulting compounds of the formulae (I-A) and (I-B) are, where appropriate, converted with the appropriate (i) solvents and/or (ii) acids or bases into their solvates, salts and/or solvates of the salts.

The compounds of the formulae (I-A) and (I-B) together form the group of the compounds of the formula (I) according to the invention.

Inert solvents for the process step (II)+(III) are, for example, alcohols such as methanol, ethanol, n-propanol, isopropanol, n-butanol or tert-butanol, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulphoxide (DMSO), N,N′-dimethylpropyleneurea (DMPU), N-methylpyrrolidone (NMP), pyridine, acetonitrile, sulpholane or else water. It is likewise possible to use mixtures of the solvents mentioned. Preference is given to tert-butanol or methanol.

Suitable bases for the process step (II)+(III) are alkali metal hydroxides such as, for example, lithium hydroxide, sodium hydroxide or potassium hydroxide, alkali metal carbonates such as lithium carbonate, sodium carbonate, potassium carbonate or caesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to potassium tert-butoxide or sodium methoxide.

The reaction (II)+(III) is generally carried out in a temperature range of from +20° C. to +150° C., preferably at from +75° C. to +100° C., optionally in a microwave. The reaction can be carried out at atmospheric, elevated or reduced pressure (for example from 0.5 to 5 bar). In general, atmospheric pressure is employed.

Process step (IV)→(V) is carried out with or without solvent. Suitable solvents are all organic solvents which are inert under the reaction conditions. The preferred solvent is dimethoxyethane.

The reaction (IV)→(V) is generally carried out in a temperature range of from +20° C. to +100° C., preferably within the range from +50° C. to +100° C., optionally in a microwave. The reaction can be performed at atmospheric, elevated or reduced pressure (for example in the range from 0.5 to 5 bar). The reaction is generally carried out at atmospheric pressure.

Suitable halogen sources in the reaction (IV)→(V) are, for example, diiodomethane, a mixture of caesium iodide, iodine and copper(I) iodide or copper(II) bromide.

Process step (IV)→(V), in the case of diiodomethane as the halogen source, is carried out with a molar ratio of 10 to 30 mol of isopentyl nitrite and 10 to 30 mol of the iodine equivalent based on 1 mol of the compound of the formula (IV).

Inert solvents for the process step (V)→(I-A) are alcohols such as methanol, ethanol, n-propanol, isopropanol, n-butanol, tert-butanol or 1,2-ethanediol, ethers such as diethyl ether, dioxane, tetrahydrofuran, glycol dimethyl ether or diethylene glycol dimethyl ether, or other solvents such as dimethylformamide (DMF), N,N′-dimethylpropyleneurea (DMPU), N-methylpyrrolidone (NMP), pyridine or else water. It is likewise possible to use mixtures of the solvents mentioned. Preference is given to DMF.

The reduction (V)→(I-A) is carried out with hydrogen in conjunction with transition metal catalysts, for example palladium (10% on activated carbon), Raney nickel or palladium hydroxide.

The reaction (V)→(I-A) is generally carried out in a temperature range of from +20° C. to +50° C. The reaction can be performed at atmospheric or elevated pressure (for example in the range from 0.5 to 5 bar). In general, atmospheric pressure is employed.

The reaction (VIII)→(IX) can be carried out in a solvent which is inert under the reaction conditions, or without solvent. The preferred solvent is sulpholane.

The reaction (VIII)→(IX) is generally carried out in a temperature range of from +70° C. to +150° C., preferably from +80° C. to +130° C., optionally in a microwave. The reaction can be performed at atmospheric or elevated pressure (for example in the range from 0.5 to 5 bar). The reaction is generally carried out at atmospheric pressure.

Especially preferably, the reaction (VIII)→(IX) is carried out without solvent in a temperature range from 0° C. to +50° C. at atmospheric pressure.

Process step (IX)→(X) is carried out in a solvent which is inert under the reaction conditions. Suitable solvents are, for example, ethers such as diethyl ether, dioxane, tetrahydrofuran, glycol dimethyl ether or diethylene glycol dimethyl ether, or other solvents such as dimethylformamide (DMF), dimethyl sulphoxide (DMSO), N,N′-dimethylpropyleneurea (DMPU), N-methylpyrrolidone (NMP), pyridine, acetonitrile or else water. It is likewise possible to use mixtures of the solvents mentioned. Preference is given to acetonitrile.

The reaction (IX)→(X) is generally carried out in a temperature range of from +20° C. to +100° C., preferably from +40° C. to +70° C., optionally in a microwave. The reaction can be performed at atmospheric or elevated pressure (for example in the range from 0.5 to 5 bar). The reaction is generally carried out at atmospheric pressure.

The cyclization (X)→(I-B) is carried out in a solvent which is inert under the reaction conditions, for example alcohols such as methanol, ethanol, n-propanol, isopropanol, n-butanol or tert-butanol, ethers such as diethyl ether, dioxane, dimethoxyethane, tetrahydrofuran (THF), glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulphoxide (DMSO), N,N′-dimethylpropyleneurea (DMPU), N-methylpyrrolidone (NMP), pyridine, acetonitrile or sulpholane. It is likewise possible to use mixtures of the solvents mentioned. Preference is given to THF.

Suitable bases for the process step (X)→(I-B) are alkali metal hydroxides such as, for example, lithium hydroxide, sodium hydroxide or potassium hydroxide, alkali metal carbonates such as lithium carbonate, sodium carbonate, potassium carbonate or caesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to potassium tert-butoxide.

The reaction (X)→(I-B) is generally carried out in a temperature range of from 0° C. to +50° C., preferably from +10° C. to +30° C., optionally in a microwave. The reaction can be performed at atmospheric or elevated pressure (for example in the range from 0.5 to 5 bar). The reaction is generally carried out at atmospheric pressure.

The cyclization to give (I-B) is preferably carried out directly in the course of the reaction (IX)→(X) without addition of further reagents.

In an alternative procedure for process [B], the conversion (VI)+(VII)→(VIII)→(IX)→(X)→(I-B) is performed without isolation of the intermediates.

The reactions (VIII)→(IX)→(X)→(I-B) are preferably carried out without isolation of the intermediates.

Inert solvents for the process step (VI)+(VII)→(VIII) are, for example, alcohols such as methanol, ethanol, n-propanol, isopropanol, n-butanol or tert-butanol, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulphoxide (DMSO), N,N′-dimethylpropyleneurea (DMPU), N-methylpyrrolidone (NMP), pyridine or acetonitrile. It is likewise possible to use mixtures of the solvents mentioned. Preference is given to methanol or ethanol.

The reaction (VI)+(VII)→(VIII) is generally carried out in a temperature range of from +50° C. to +120° C., preferably from +50° C. to +100° C., optionally in a microwave. The reaction can be performed at atmospheric or elevated pressure (for example in the range from 0.5 to 5 bar). The reaction is generally carried out at atmospheric pressure.

Inert solvents for the process step (II)→(VI) are, for example, alcohols such as methanol, ethanol, n-propanol, isopropanol, n-butanol or tert-butanol, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulphoxide (DMSO), N,N′-dimethylpropyleneurea (DMPU), N-methylpyrrolidone (NMP), pyridine or acetonitrile. It is likewise possible to use mixtures of the solvents mentioned. Preference is given to ethanol.

Suitable bases for the process step (II)→(VI) are alkali metal hydroxides such as, for example, lithium hydroxide, sodium hydroxide or potassium hydroxide, alkali metal carbonates such as lithium carbonate, sodium carbonate, potassium carbonate or caesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to triethylamine.

The reaction (II)→(VI) is generally carried out in a temperature range of from 0° C. to +60° C., preferably from +10° C. to +30° C. The reaction can be performed at atmospheric or elevated pressure (for example in the range from 0.5 to 5 bar). In general, atmospheric pressure is employed.

The preparation processes described can be illustrated by way of example by the following synthesis schemes (Schemes 1 and 2):

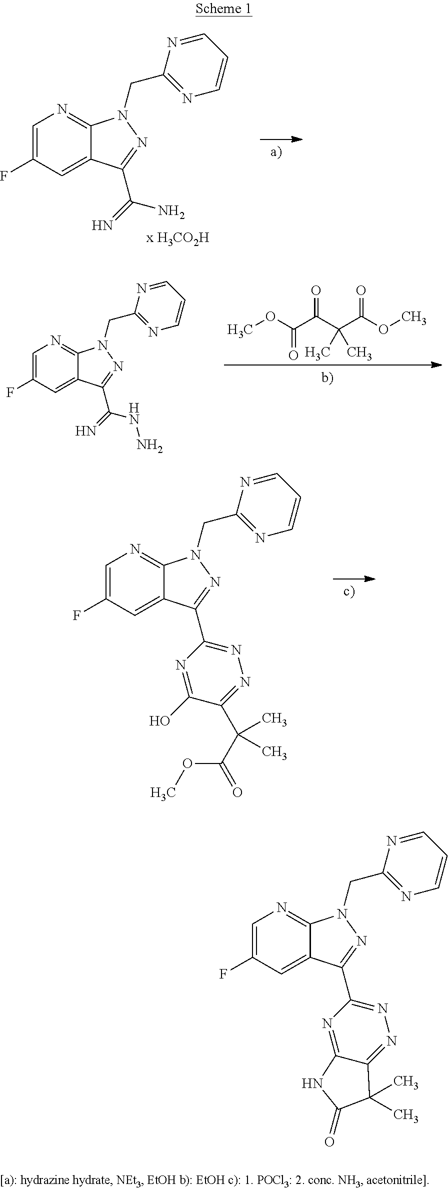

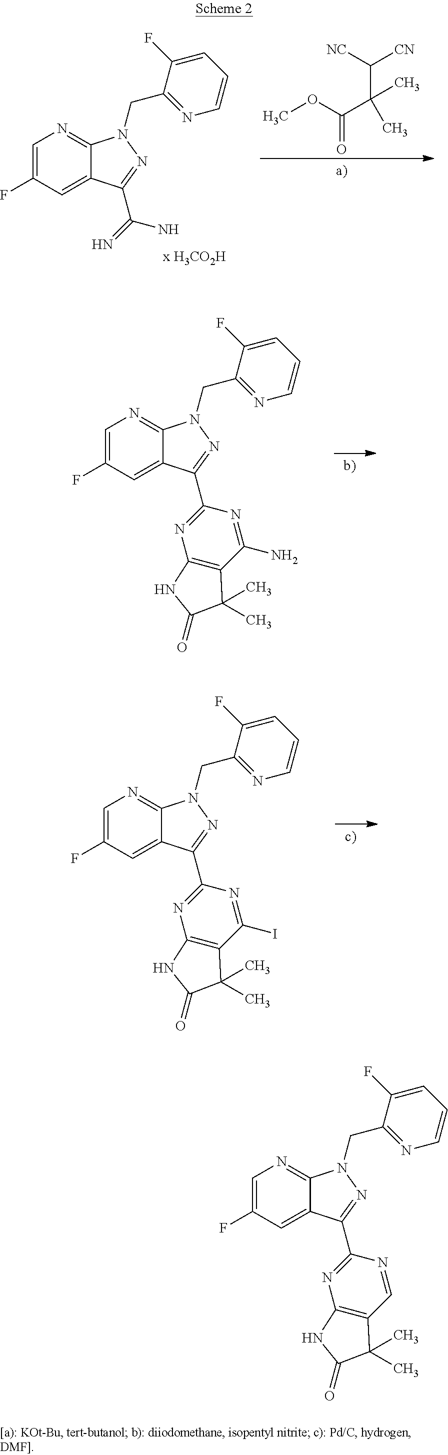 [SMILES: N=C(N)c1nn(Cc2ncccc2F)c2ncc(F)cc12 ; COC(=O)C(C)(C)C(C#N)C#N ; CC1(C)C(=O)Nc2nc(-c3nn(Cc4ncccc4F)c4ncc(F)cc34)nc(N)c21 ; CC1(C)C(=O)Nc2nc(-c3nn(Cc4ncccc4F)c4ncc(F)cc34)nc(I)c21 ; CC1(C)C(=O)Nc2nc(-c3nn(Cc4ncccc4F)c4ncc(F)cc34)ncc21]

Further compounds according to the invention can optionally also be prepared by conversions of functional groups of individual substituents, especially those listed for L and R³, proceeding from compounds of the formula (I) obtained by the above processes. These conversions are performed by customary methods known to those skilled in the art and include, for example, reactions such as nucleophilic and electrophilic substitutions, oxidations, reductions, hydrogenations, transition metal-catalyzed coupling reactions, eliminations, alkylation, amination, esterification, ester cleavage, etherification, ether cleavage, formation of carbonamides, and introduction and removal of temporary protective groups.

In an alternative process, the preparation of the compounds of the formula (I) according to the invention can take place by reversing the order of the reaction steps using protective group chemistry, as shown by way of example in the synthesis scheme below (Scheme 6):

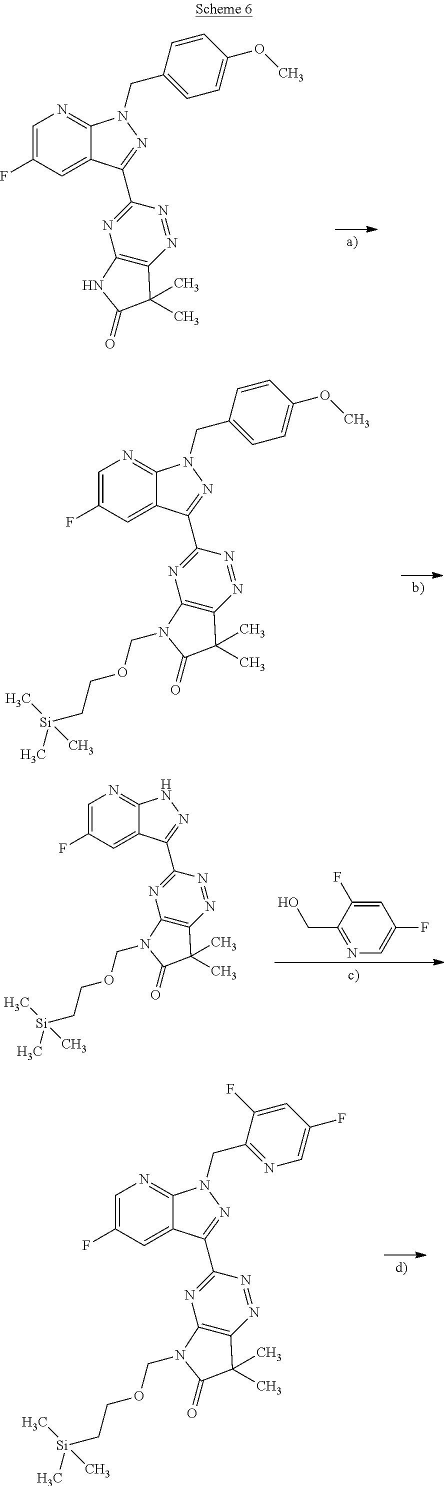

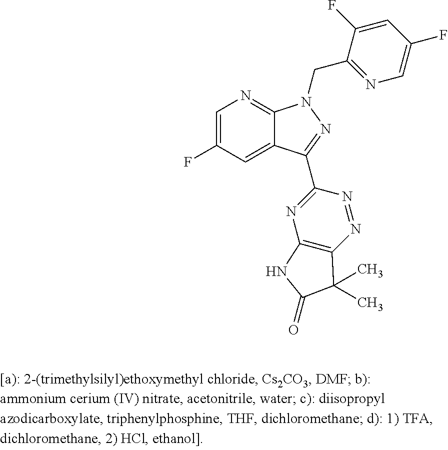 [SMILES: CC1(C)C(=O)Nc2nc(-c3nn(Cc4ncc(F)cc4F)c4ncc(F)cc34)nnc21]

The compounds of the formula (II) can be prepared by cyclizing a compound of the formula (XI)

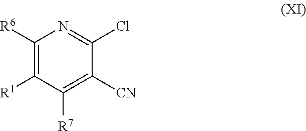

in which R¹, R⁶ and R⁷ have the meaning given above in an inert solvent with hydrazine hydrate to give the compound of the formula (XII)

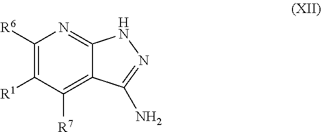

in which R¹, R⁶ and R⁷ have the meaning given above
then reacting the latter, in an inert solvent in the presence of a suitable Lewis acid, first with isopentyl nitrite to give the corresponding diazonium salt, and then converting the latter directly with sodium iodide into the compound of the formula (XIII)

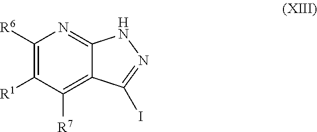

in which R¹, R⁶ and R⁷ have the meaning given above this is subsequently converted in an inert solvent in the presence of a suitable base with the compound of the formula (XIV)

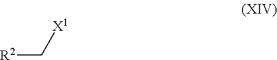

in which R² has the meaning given above and
X¹ represents a suitable leaving group, for example halogen, tosylate or mesylate, or represents hydroxy,
   into a compound of the formula (XV)

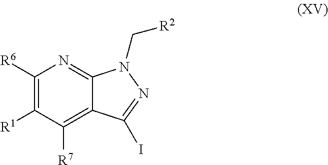

in which R¹, R², R⁶ and R⁷ each have the meanings given above, this is then reacted in an inert solvent with copper cyanide to give a compound of the formula (XVI)

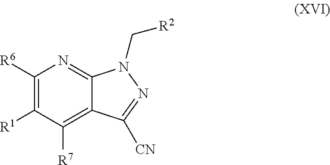

in which R¹, R², R⁶ and R⁷ each have the meanings given above, and this is finally reacted under acidic conditions with an ammonia equivalent.

Inert solvents for the process step (XI)→(XII) are alcohols such as methanol, ethanol, n-propanol, isopropanol, n-butanol, tert-butanol or 1,2-ethanediol, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulphoxide (DMSO), N,N′-dimethylpropyleneurea (DMPU), N-methylpyrrolidone (NMP), pyridine, acetonitrile or else water. It is likewise possible to use mixtures of the solvents mentioned. 1,2-Ethanediol is preferred.

The reaction (XI)→(XII) is generally carried out in a temperature range of from +60° C. to +200° C., preferably from +120° C. to +180° C. The reaction can be carried out at atmospheric, elevated or reduced pressure (for example from 0.5 to 5 bar). In general, atmospheric pressure is employed.

Inert solvents for the reaction (XII)→(XIII) are, for example, halohydrocarbons such as dichloromethane, trichloromethane, tetrachloromethane, trichloroethylene or chlorobenzene, ethers such as diethyl ether, dioxane, tetrahydrofuran, glycol dimethyl ether or diethylene glycol dimethyl ether, or other solvents such as dimethylformamide (DMF), dimethyl sulphoxide (DMSO), N,N′-dimethylpropyleneurea (DMPU), N-methylpyrrolidone (NMP), pyridine or acetonitrile. Preference is given to DMF.

Suitable Lewis acids for the process step (XII)→(XIII) are boron trifluoride/diethyl ether complex, cerium(IV) ammonium nitrate (CAN), tin(II) chloride, lithium perchlorate, zinc(II) chloride, indium(III) chloride or indium(III) bromide. Preference is given to boron trifluoride/diethyl ether complex.

The reaction (XII)→(XIII) is generally carried out in a temperature range from −78° C. to +40° C., preferably at from 0° C. to +20° C. The reaction can be carried out at atmospheric, elevated or reduced pressure (for example from 0.5 to 5 bar). In general, atmospheric pressure is employed.

Inert solvents for the reaction (XIII)+(XIV)→(XV) are, for example, halohydrocarbons such as dichloromethane, trichloromethane, tetrachloromethane, trichloroethylene or chlorobenzene, ethers such as diethyl ether, dioxane, tetrahydrofuran, glycol dimethyl ether or diethylene glycol dimethyl ether, or other solvents such as dimethylformamide (DMF), dimethyl sulphoxide (DMSO), N,N′-dimethylpropyleneurea (DMPU), N-methylpyrrolidone (NMP), pyridine, acetonitrile. Preference is given to DMF.

Suitable bases for the process step (XIII)+(XIV)→(XV) are alkali metal hydrides such as potassium hydride or sodium hydride, alkali metal carbonates such as lithium carbonate, sodium carbonate, potassium carbonate or caesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, amides such as sodium amide, lithium bis(trimethylsilyl)amide, sodium bis(trimethylsilyl)amide or potassium bis(trimethylsilyl)amide or lithium diisopropylamide, organometallic compounds such as butyllithium or phenyllithium, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to caesium carbonate.

The reaction (XIII)+(XIV)→(XV) is generally carried out in a temperature range of from 0° C. to +60° C., preferably from +10° C. to +25° C. The reaction can be carried out at atmospheric, elevated or reduced pressure (for example from 0.5 to 5 bar). In general, atmospheric pressure is employed.

If X² represents hydroxy, the reaction (XIII)+(XIV)→(XV) is carried out under Mitsunobu conditions. The Mitsunobu reaction is carried out using triphenylphosphine, or tri-n-butylphosphine, 1,2-bis(diphenylphosphino)ethane (DPPE), diphenyl(2-pyridyl)phosphine (Ph2P-Py), (p-dimethylaminophenyl)diphenylphosphine (DAP-DP), tris(4-dimethylaminophenyl)phosphine (tris-DAP), and a suitable dialkyl azodicarboxylate, for example diethyl azodicarboxylate (DEAD), diisopropyl azodicarboxylate (DIAD), di-tert-butyl azodicarboxylate, N,N,N′ N′-tetramethylazodicarboxamide (TMAD), 1,1′-(azodicarbonyl)dipiperidine (ADDP) or 4,7-dimethyl-3,5,7-hexahydro-1,2,4,7-tetrazocine-3,8-dione (DHTD). Preference is given to using triphenylphosphine and diisopropyl azodicarboxylate (DIAD), or a suitable azodicarboxamide such as, for example, N,N,N′,N′-tetramethyldiazene-1,2-dicarboxamide.

Inert solvents for the Mitsunobu reaction (XIII)+(XIV)→(XV) are, for example, ethers such as tetrahydrofuran, diethyl ether, hydrocarbons such as benzene, toluene, xylene, halohydrocarbons such as dichloromethane, dichloroethane or other solvents such as acetonitrile, DMF or NMP. It is also possible to use mixtures of the solvents mentioned. Preference is given to using THF.

The Mitsunobu reaction (XIII)+(XIV)→(XV) is generally carried out in a temperature range of from −78° C. to +180° C., preferably from 0° C. to +50° C., optionally in a microwave. The conversions can be performed at atmospheric, elevated or reduced pressure (for example from 0.5 to 5 bar).

Inert solvents for the process step (XV)→(XVI) are, for example, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulphoxide (DMSO), N,N′-dimethylpropyleneurea (DMPU), N-methylpyrrolidone (NMP), pyridine or acetonitrile. It is likewise possible to use mixtures of the solvents mentioned. Preference is given to DMSO.

The reaction (XV)→(XVI) is generally carried out in a temperature range of from +20° C. to +180° C., preferably at from +100° C. to +160° C., optionally in a microwave. The reaction can be carried out at atmospheric, elevated or reduced pressure (for example from 0.5 to 5 bar). In general, atmospheric pressure is employed.

The reaction (XVI)→(II) is carried out using methods known to the person skilled in the art in a two-step process, initially with formation of the imino ester using sodium methoxide in methanol at from 0° C. to +40° C. and then nucleophilic addition of an ammonia equivalent such as, for example, ammonia or ammonium chloride in a suitable acid with formation of the amidine (III) at from +50 to +150° C.

Suitable acids for the formation of the amidine (II) are inorganic acids, for example hydrogen chloride/hydrochloric acid, sulphuric acid, polyphosphoric acid or phosphoric acid, or organic acids, for example acetic acid, trifluoroacetic acid or formic acid. Preference is given to using hydrochloric acid or acetic acid.

The preparation process described can be illustrated in an exemplary manner by the synthesis scheme below (Scheme 3):

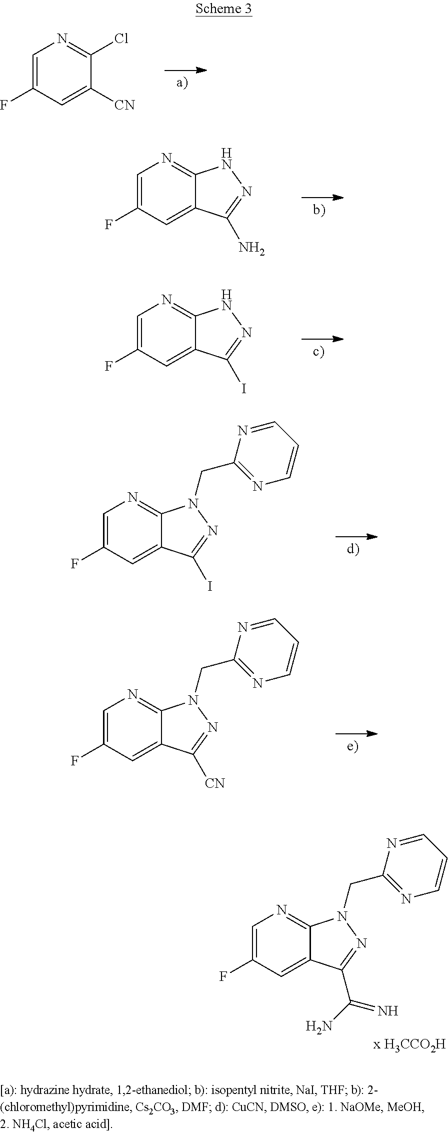

Alternatively, the preparation of the compounds of the formula (II) is carried out as shown in the synthesis scheme below (Scheme 4):

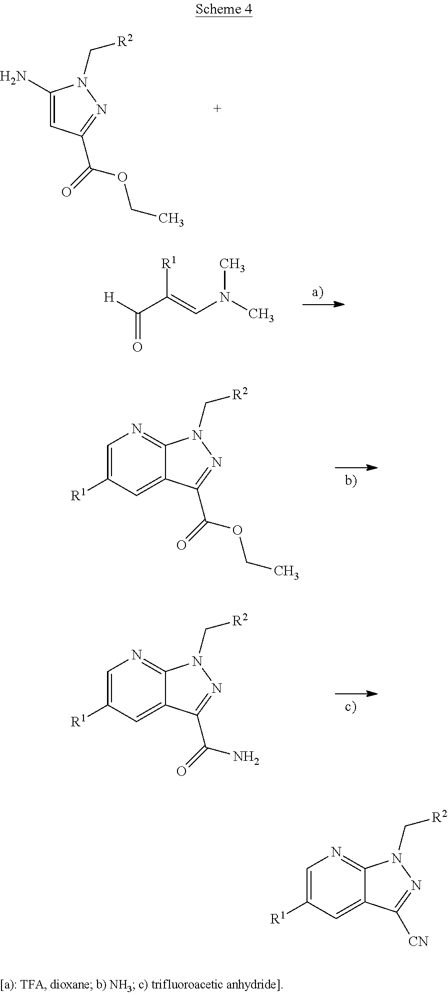

The compound of the formula (XI) is known from the literature [cf., for example, Winn M., J. Med. Chem. 1993, 36, 2676-7688; EP 634 413-A1; CN 1613849-A; EP 1626045-A1; WO 2009/018415] and can be prepared in analogy to literature processes or as shown in the synthesis scheme below (Scheme 5):

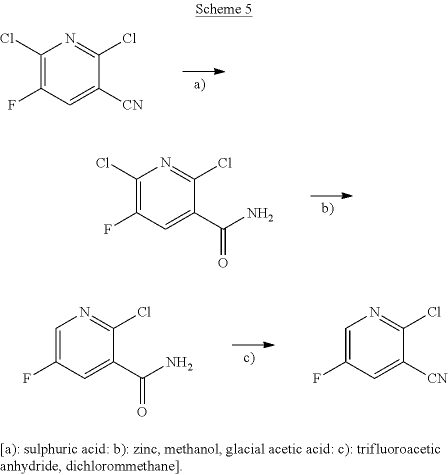 [SMILES: N#Cc1cc(F)c(Cl)nc1Cl ; NC(=O)c1cc(F)c(Cl)nc1Cl ; NC(=O)c1cc(F)cnc1Cl ; N#Cc1cc(F)cnc1Cl]

The compounds of the formulae (III) and (VII) are commercially available, known from the literature or can be prepared in analogy to processes known from the literature.

The compounds according to the invention act as stimulators of soluble guanylate cyclase and have an identical or improved therapeutic profile compared to the compounds known from the prior art, such as, for example, with respect to their in vivo properties such as, for example, their pharmacokinetic and pharmacodynamic behaviour and/or their metabolism profile and/or their dose-activity relationship. They are therefore suitable for the treatment and/or prophylaxis of diseases in man and animals.

The compounds according to the invention cause vasorelaxation and inhibition of platelet aggregation, and lead to a decrease in blood pressure and to a rise in coronary blood flow. These effects are mediated via direct stimulation of soluble guanylate cyclase and intracellular cGMP increase. Moreover, the compounds according to the invention enhance the effect of substances increasing the cGMP concentration, such as, for example, EDRF (endothelium-derived relaxing factor), NO donors, protoporphyrin IX, arachidonic acid or phenylhydrazine derivatives.

The compounds according to the invention are suitable for the treatment and/or prophylaxis of cardiovascular, pulmonary, thromboembolic and fibrotic disorders.

Accordingly, the compounds according to the invention can be used in medicaments for the treatment and/or prophylaxis of cardiovascular disorders such as, for example, hypertension, acute and chronic heart failure, coronary heart disease, stable and unstable angina pectoris, peripheral and cardiac vascular disorders, arrhythmias, atrial and ventricular arrhythmias and impaired conduction such as, for example, atrioventricular blocks degrees I-III (AB block supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for the treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation such as, for example, pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for the treatment and/or prophylaxis of erectile dysfunction and female sexual dysfunction.

In the context of the present invention, the term “heart failure” also encompasses both acute and chronic forms of heart failure, and also more specific or related types of disease, such as acute decompensated heart failure, right heart failure, left heart failure, global failure, ischemic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure, and acute phases of worsening of existing chronic heart failure (worsening heart failure).

In addition, the compounds according to the invention can also be used for the treatment and/or prophylaxis of arteriosclerosis, impaired lipid metabolism, hypolipoproteinemias, dyslipidemias, hypertriglyceridemias, hyperlipidemias, hypercholesterolemias, abetalipoproteinemia, sitosterolemia, xanthomatosis, Tangier disease, adiposity, obesity and of combined hyperlipidemias and metabolic syndrome.

The compounds according to the invention can additionally be used for the treatment and/or prophylaxis of primary and secondary Raynaud's phenomenon, of microcirculation impairments, claudication, peripheral and autonomic neuropathies, diabetic microangiopathies, diabetic retinopathy, diabetic ulcers on the extremities, gangrene, CREST syndrome, erythematosis, onychomycosis, rheumatic disorders and for promoting wound healing.

The compounds according to the invention are furthermore suitable for treating urological disorders such as, for example, benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic overactive bladder (OAB) and (IC), incontinence (UI) such as, for example, mixed urinary incontinence, urge urinary incontinence, stress urinary incontinence or overflow urinary incontinence (MUI, UUI, SUI, OUI), pelvic pain, benign and malignant disorders of the organs of the male and female urogenital system.

The compounds according to the invention are furthermore suitable for the treatment and/or prophylaxis of kidney disorders, in particular of acute and chronic renal insufficiency and acute and chronic renal failure. In the context of the present invention, the term renal insufficiency comprises both acute and chronic manifestations thereof, as well as underlying or related kidney diseases such as renal hypoperfusion, intradialytic hypotension, obstructive uropathy, glomerulopathies, glomerulonephritis, acute glomerulonephritis, glomerulosclerosis, tubulointerstitial diseases, nephropathic diseases such as primary and congenital kidney disease, nephritis, immunological kidney diseases such as kidney graft rejection and immunocomplex-induced kidney diseases, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome, which can be characterized diagnostically for example by abnormally reduced creatinine and/or water excretion, abnormally raised blood concentrations of urea, nitrogen, potassium and/or creatinine, altered activity of renal enzymes such as, for example, glutamyl synthetase, altered urine osmolarity or urine volume, increased microalbuminuria, macroalbuminuria, lesions on glomerulae and arterioles, tubular dilatation, hyperphosphataemia and/or need for dialysis. The present invention also encompasses the use of the compounds according to the invention for treatment and/or prophylaxis of sequelae of renal insufficiency, for example pulmonary oedema, heart failure, uraemia, anaemia, electrolyte disturbances (for example hyperkalaemia, hyponatraemia) and disturbances in bone and carbohydrate metabolism.

Furthermore, the compounds according to the invention are also suitable for the treatment and/or prophylaxis of asthmatic disorders, pulmonary arterial hypertension (PAH) and other forms of pulmonary hypertension (PH) including left-heart disease, HIV, sickle cell anaemia, thromboembolisms (CTEPH), sarcoidosis, COPD or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelinisation, multiple sclerosis, thalamic degeneration, Creutzfeld-Jacob dementia, HIV dementia, schizophrenia with dementia or Korsakoff's psychosis. They are also suitable for the treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

Furthermore, the compounds according to the invention are also suitable for regulating cerebral blood flow and are thus effective agents for control of migraine. They are also suitable for prophylaxis and control of sequelae of cerebral infarction (cerebral apoplexy) such as stroke, cerebral ischaemia and craniocerebral trauma. The compounds according to the invention can likewise be employed for controlling states of pain and tinnitus.

In addition, the compounds according to the invention have antiinflammatory action and can therefore be used as antiinflammatory agents for the treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin diseases and inflammatory eye diseases.

Furthermore, the compounds according to the invention can also be used for the treatment and/or prophylaxis of autoimmune diseases.

The compounds according to the invention are furthermore suitable for the treatment and/or prophylaxis of fibrotic disorders of the internal organs such as, for example, the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarkoidosis).

The compounds according to the invention are furthermore suitable for controlling postoperative scarring, for example as a result of glaucoma operations.

The compounds according to the invention can also be used cosmetically for ageing and keratinized skin.

Moreover, the compounds according to the invention are suitable for the treatment and/or prophylaxis of hepatitis, neoplasms, osteoporosis, glaucoma and gastroparesis.

The present invention further provides for the use of the inventive compounds for treatment and/or prophylaxis of disorders, especially of the aforementioned disorders.

The present invention further provides the use of the compounds according to the invention for the treatment and/or prophylaxis of heart failure, angina pectoris, hypertension, pulmonary hypertension, ischaemias, vascular disorders, kidney failure, thromboembolic disorders, fibrotic disorders and arteriosclerosis.

The present invention further provides the compounds according to the invention for use in a method for the treatment and/or prophylaxis of heart failure, angina pectoris, hypertension, pulmonary hypertension, ischaemias, vascular disorders, kidney failure, thromboembolic disorders, fibrotic disorders and arteriosclerosis.

The present invention further provides for the use of the compounds according to the invention for production of a medicament for treatment and/or prophylaxis of disorders, especially of the aforementioned disorders.

The present invention further provides for the use of the compounds according to the invention for producing a medicament for treatment and/or prophylaxis of heart failure, angina pectoris, hypertension, pulmonary hypertension, ischamias, vascular disorders, kidney failure, thromboembolic disorders, fibrotic disorders and arteriosclerosis.

The present invention further provides a method for treatment and/or prophylaxis of disorders, in particular the disorders mentioned above, using an effective amount of at least one of the compounds according to the invention.

The present invention further provides a method for treatment and/or prophylaxis of heart failure, angina pectoris, hypertension, pulmonary hypertension, ischaemias, vascular disorders, kidney failure, thromboembolic disorders, fibrotic disorders and arteriosclerosis using an effective amount of at least one of the compounds according to the invention.

The compounds according to the invention can be employed alone or, if required, in combination with other active compounds. The present invention further provides medicaments comprising at least one of the compounds according to the invention and one or more further active compounds, especially for the treatment and/or prophylaxis of the aforementioned disorders. Preferred examples of suitable active ingredient combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, in particular PDE 5 inhibitors such as sildenafil, vardenafil and tadalafil;
agents having an antithrombotic effect, for example and with preference from the group of platelet aggregation inhibitors, of anticoagulants or of profibrinolytic substances;
active compounds which lower blood pressure, for example and preferably from the group of calcium antagonists, angiotensin AII antagonists, ACE inhibitors, endothelin antagonists, renin inhibitors, alpha-receptor blockers, beta-receptor blockers, mineralocorticoid receptor antagonists, and of diuretics; and/or
active compounds which alter lipid metabolism, for example and with preference from the group of thyroid receptor agonists, cholesterol synthesis inhibitors such as, by way of example and preferably, HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

Agents having antithrombotic activity preferably mean compounds from the group of platelet aggregation inhibitors, of anticoagulants or of profibrinolytic substances.

In a preferred embodiment of the invention, the inventive compounds are administered in combination with a platelet aggregation inhibitor, by way of example and with preference aspirin, clopidogrel, ticlopidin or dipyridamol.

In a preferred embodiment of the invention, the compounds according to the invention are administered in combination with a thrombin inhibitor, by way of example and with preference ximelagatran, dabigatran, melagatran, bivalirudin or clexane.

In a preferred embodiment of the invention, the inventive compounds are administered in combination with a GPIIb/IIIa antagonist, by way of example and with preference tirofiban or abciximab.

In a preferred embodiment of the invention, the inventive compounds are administered in combination with a factor Xa inhibitor, preferred examples being rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the inventive compounds are administered in combination with a vitamin K antagonist, by way of example and with preference coumarin.

Hypotensive agents are preferably understood to mean compounds from the group of calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the inventive compounds are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the inventive compounds are administered in combination with a beta receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with an angiotensin AII antagonist, by way of example and with preference losartan, candesartan, valsartan, telmisartan or embursartan.

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the inventive compounds are administered in combination with an endothelin antagonist, by way of example and with preference bosentan, darusentan, ambrisentan or sitaxsentan.

In a preferred embodiment of the invention, the compounds according to the invention are administered in combination with a renin inhibitor, by way of example and with preference aliskiren, SPP-600 or SPP-800.

In a preferred embodiment of the invention, the inventive compounds are administered in combination with a mineralocorticoid receptor antagonist, by way of example and with preference spironolactone or eplerenone.

In a preferred embodiment of the invention, the compounds according to the invention are administered in combination with a loop diuretic such as, for example, furosemide, torasemide, bumetanide and piretanide, with potassium-sparing diuretics such as, for example, amiloride and triamterene, with aldosterone antagonists such as, for example, spironolactone, potassium canrenoate and eplerenone and also thiazide diuretics such as, for example, hydrochlorothiazide, chlorthalidone, xipamide and indapamide.

Agents which modify lipid metabolism are preferably understood to mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and lipoprotein(a) antagonists.

In a preferred embodiment of the invention, the compounds according to the invention are administered in combination with a CETP inhibitor, by way of example and with preference dalcetrapib, BAY 60-5521, anacetrapib oder CETP vaccine (CETi-1).

In a preferred embodiment of the invention, the inventive compounds are administered in combination with a thyroid receptor agonist, by way of example and with preference D-thyroxin, 3,5,3′-triiodothyronin (T3), CGS 23425 or axitirome (CGS 26214).

In a preferred embodiment of the invention, the inventive compounds are administered in combination with a HMG-CoA reductase inhibitor from the class of the statins, by way of example and with preference lovastatin, simvastatin, pravastatin, fluvastatin, atorvastatin, rosuvastatin or pitavastatin.

In a preferred embodiment of the invention, the inventive compounds are administered in combination with a squalene synthesis inhibitor, by way of example and with preference BMS-188494 or TAK-475.

In a preferred embodiment of the invention, the compounds according to the invention are administered in combination with an ACAT inhibitor, by way of example and with preference avasimibe, melinamide, pactimibe, eflucimibe or SMP-797.

In a preferred embodiment of the invention, the compounds according to the invention are administered in combination with an MTP inhibitor, by way of example and with preference implitapide, BMS-201038, R-103757 or ITT-130.

In a preferred embodiment of the invention, the inventive compounds are administered in combination with a PPAR-gamma agonist, by way of example and with preference pioglitazone or rosiglitazone.

In a preferred embodiment of the invention, the inventive compounds are administered in combination with a PPAR-delta agonist, by way of example and with preference GW 501516 or BAY 68-5042.

In a preferred embodiment of the invention, the inventive compounds are administered in combination with a cholesterol absorption inhibitor, by way of example and with preference ezetimibe, tiqueside or pamaqueside.

In a preferred embodiment of the invention, the compounds according to the invention are administered in combination with a lipase inhibitor, a preferred example being orlistat.

In a preferred embodiment of the invention, the inventive compounds are administered in combination with a polymeric bile acid adsorbent, by way of example and with preference cholestyramine, colestipol, colesolvam, CholestaGel or colestimide.

In a preferred embodiment of the invention, the compounds according to the invention are administered in combination with a bile acid reabsorption inhibitor, by way of example and with preference ASBT(=IBAT) inhibitors, for example AZD-7806, S-8921, AK-105, BARI-1741, SC-435 or SC-635.

In a preferred embodiment of the invention, the inventive compounds are administered in combination with a lipoprotein(a) antagonist, by way of example and with preference gemcabene calcium (CI-1027) or nicotinic acid.

The present invention further provides medicaments which comprise at least one compound according to the invention, typically together with one or more inert nontoxic pharmaceutically suitable auxiliaries, and for the use thereof for the aforementioned purposes.

The inventive compounds can act systemically and/or locally. For this purpose, they can be administered in a suitable manner, for example by the oral, parenteral, pulmonal, nasal, sublingual, lingual, buccal, rectal, dermal, transdermal, conjunctival, otic route, or as an implant or stent.

The compounds according to the invention can be administered in administration forms suitable for these administration routes.

Administration forms which function according to the prior art, release the compounds according to the invention rapidly and/or in a modified manner and contain the compounds according to the invention in crystalline and/or amorphized and/or dissolved form are suitable for oral administration, such as e.g. tablets (non-coated or coated tablets, for example with enteric coatings or coatings that dissolve in a delayed manner or are insoluble and control the release of the compound according to the invention), tablets or films/oblates, films/lyophilisates or capsules which disintegrate rapidly in the oral cavity (for example hard or soft gelatine capsules), sugar-coated tablets, granules, pellets, powders, emulsions, suspensions, aerosols or solutions.

Parenteral administration can bypass an absorption step (e.g. intravenously, intraarterially, intracardially, intraspinally or intralumbally) or include an absorption (e.g. intramuscularly, subcutaneously, intracutaneously, percutaneously or intraperitoneally). Administration forms suitable for parenteral administration include preparations for injection and infusion in the form of solutions, suspensions, emulsions, lyophilisates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds according to the invention can be converted to the administration forms mentioned. This can be accomplished in a manner known per se by mixing with inert nontoxic pharmaceutically suitable auxiliaries. These auxiliaries include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavour and/or odour correctors.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

In spite of this, it may be necessary to deviate from the amounts specified, specifically depending on body weight, administration route, individual behaviour towards the active ingredient, type of formulation, and time or interval of administration. For instance, less than the aforementioned minimum amount may be sufficient in some cases, while the upper limit mentioned has to be exceeded in other cases. In the case of administration of greater amounts, it may be advisable to divide them into several individual doses over the day.

The working examples which follow illustrate the invention. The invention is not restricted to the examples.

The percentages in the tests and examples which follow are, unless stated otherwise, percentages by weight; parts are parts by weight. Solvent ratios, dilution ratios and concentration figures for liquid/liquid solutions are each based on volume.

### A. EXAMPLES

### Abbreviations and Acronyms

aq. aqueous solution
calc. calculated
br s broad singlet (in NMR)
DCI direct chemical ionization (in MS)
DMF dimethylformamide
DMSO dimethyl sulphoxide
eq. equivalent(s)
ESI electrospray ionization (in MS)
Et ethyl
fnd. found
h hour(s)
HPLC high-pressure, high-performance liquid chromatography
HRMS high-resolution mass spectrometry
conc. concentrated
LC-MS liquid chromatography-coupled mass spectrometry
Me methyl
min minute(s)
MS mass spectrometry
NMR nuclear magnetic resonance spectrometry
Ph phenyl
RT room temperature
Rₜ retention time (in HPLC)
THF tetrahydrofuran
UV ultraviolet spectrometry
v/v ratio by volume (of a solution)

### LC/MS Methods:

### Method 1:

MS instrument type: Waters ZQ; HPLC instrument type: Agilent 1100 Series; UV DAD; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% formic acid; gradient: 0.0 min 100% A→3.0 min 10% A→4.0 min 10% A→4.1 min 100% A (flow rate 2.5 ml/min); oven: 55° C.; flow rate: 2 ml/min; UV detection: 210 nm.

### Method 2:

Instrument: Waters ACQUITY SQD UPLC System; column: Waters Acquity UPLC HSS T3 1.8μ 50×1 mm; mobile phase A: 1 l of water+0.25 ml of 99% strength formic acid, mobile phase B: 1 l of acetonitrile+0.25 ml of 99% formic acid; gradient: 0.0 min 90% A→1.2 min 5% A→2.0 min 5% A; oven: 50° C.; flow rate: 0.40 ml/min; UV detection: 210-400 nm.

### Method 3:

Instrument: Waters ACQUITY SQD UPLC System; column: Waters Acquity UPLC HSS T3 1.8μ 30×2 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.60 ml/min; UV detection: 208-400 nm.

### Method 4:

Instrument: Micromass Quattro Premier with Waters UPLC Acquity; column: Thermo Hypersil GOLD 1.9μ 50×1 mm; mobile phase A: 1 l of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

### Method 5:

MS instrument: Waters (Micromass) Quattro Micro; HPLC instrument: Agilent 1100 series; column: YMC-Triart C18 3μ 50×3 mm; mobile phase A: 1 l of water+0.01 mol of ammonium carbonate, mobile phase B: 1 l of acetonitrile; gradient: 0.0 min 100% A→2.75 min 5% A→4.5 min 5% A; oven: 40° C.; flow rate: 1.25 ml/min; UV detection: 210 nm.

### Starting Compounds and Intermediates

### Example 1A

### 2,6-Dichloro-5-fluoronicotinamide

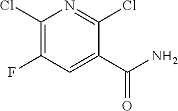 [SMILES: NC(=O)c1cc(F)c(Cl)nc1Cl]

A suspension of 25 g (130.90 mmol) of 2,6-dichloro-5-fluoro-3-cyanopyridine in conc. sulphuric acid (125 ml) was stirred at 60-65° C. for 1 h. After cooling to RT, the contents of the flask were poured into ice-water and extracted three times with ethyl acetate (100 ml each time). The combined organic phases were washed with water (100 ml) and then with saturated aqueous sodium bicarbonate solution (100 ml), dried and concentrated on a rotary evaporator. The material obtained was dried under a high vacuum.

Yield: 24.5 g (90% of theory)

¹H NMR (400 MHz, DMSO-d₆): δ=7.95 (br s, 1H), 8.11 (br s, 1H), 8.24 (d, 1H).

### Example 2A

### 2-Chloro-5-fluoronicotinamide

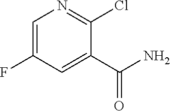 [SMILES: NC(=O)c1cc(F)cnc1Cl]

At RT, 44 g (210.58 mmol) of 2,6-dichloro-5-fluoronicotinamide were added to a suspension of 21.9 g (335.35 mmol) of zinc in methanol (207 ml). Acetic acid (18.5 ml) was then added, and the mixture was heated with stirring at reflux for 24 h. The contents of the flask were then decanted from the zinc, and ethyl acetate (414 ml) and saturated aqueous sodium hydrogen carbonate solution (414 ml) were added, followed by intense extractive stirring. Subsequently the reaction mixture was filtered with suction through kieselguhr and the filter product was washed three times with ethyl acetate (517 ml each time). The organic phase was separated off and the aqueous phase was washed with ethyl acetate (258 ml). The combined organic phases were washed once with saturated aqueous sodium bicarbonate solution (414 ml), dried and concentrated under reduced pressure. Dichloromethane (388 ml) was added to the crystals obtained in this manner, and the mixture was stirred for 20 min. The mixture was once more filtered off with suction, washed with diethyl ether and sucked dry.

Yield: 20.2 g (53% of theory)

¹H NMR (400 MHz, DMSO-d₆): δ=7.87 (br s, 1H), 7.99 (dd, 1H), 8.10 (br s, 1H), 8.52 (d, 1H).

### Example 3A

### 2-Chloro-5-fluoronicotinonitrile

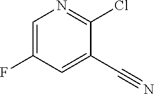

81.2 ml (582.25 mmol) of triethylamine were added to a suspension of 46.2 g (264.66 mmol) of 2-chloro-5-fluoronicotinamide in dichloromethane (783 ml), and the mixture was cooled to 0° C. Then, with stirring, 41.12 ml (291.13 mmol) of trifluoroacetic anhydride were added slowly dropwise, and the mixture was stirred at 0° C. for 1.5 h. The reaction solution was subsequently washed twice with saturated aqueous sodium bicarbonate solution (391 ml each time), dried and concentrated under reduced pressure.

Yield: 42.1 g (90% of theory)

¹H NMR (400 MHz, DMSO-d₆): δ=8.66 (dd, 1H), 8.82 (d, 1H).

### Example 4A

### 5-Fluoro-1H-pyrazolo[3,4-b]pyridine-3-amine

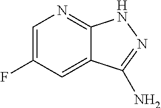 [SMILES: Nc1n[nH]c2ncc(F)cc12]

A suspension of 38.5 g (245.93 mmol) of 2-chloro-5-fluoronicotinonitrile was introduced in 1,2-ethanediol (380 ml), and hydrazine hydrate (119.6 ml, 2.459 mol) was then added. The mixture was heated under reflux with stirring for 4 h. The product precipitated on cooling. Water (380 ml) was added to the yellow crystals, and the mixture was subjected to extractive stirring at RT for 10 min. The suspension was then filtered with suction over a frit, and the filter product was washed with water (200 ml) and with −10° C. cold THF (200 ml). The residue was dried under a high vacuum over phosphorus pentoxide.

Yield: 22.8 g (61% of theory)

²H NMR (400 MHz, DMSO-d₆): δ=5.54 (s, 2H), 7.96 (dd, 1H), 8.38 (m, 1H), 12.07 (m, 1H).

### Example 5A

### 5-Fluoro-3-iodo-1H-pyrazolo[3,4-b]pyridine

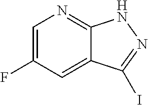 [SMILES: Fc1cnc2[nH]nc(I)c2c1]

10 g (65.75 mmol) of 5-fluoro-1H-pyrazolo[3,4-b]pyridine-3-amine were initially charged in THF (329 ml), and the mixture was cooled to 0° C. 16.65 ml (131.46 mmol) of boron trifluoride diethyl ether complex were then added slowly. The reaction mixture was cooled further to −10° C. A solution of 10.01 g (85.45 mmol) of isopentyl nitrite in THF (24.39 ml) was then added slowly, and the mixture was stirred for a further 30 min. The mixture was diluted with cold diethyl ether (329 ml) and the resulting solid was isolated by filtration. The diazonium salt thus prepared was added a little at a time to a solution at 0° C. of 12.81 g (85.45 mmol) of sodium iodide in acetone (329 ml), and the mixture was stirred at RT for 30 min. The reaction mixture was poured into ice-water (1.8 l) and extracted twice with ethyl acetate (487 ml each time). The collected organic phases were washed with saturated aqueous sodium chloride solution (244 ml), dried, filtered and concentrated. This gave 12.1 g (86% pure, 60% of theory) of the desired compound as a brown solid. The crude product was converted without further purification.

LC-MS (Method 1): Rₜ=1.68 min; MS (ESIpos): m/z=264 (M+H)⁺

### Example 6A

### 5-Fluoro-3-iodo-1-(pyrimidin-2-ylmethyl)-1H-pyrazolo[3,4-b]pyridine

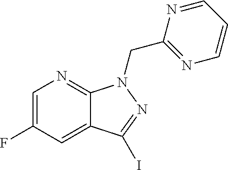 [SMILES: Fc1cnc2c(c1)c(I)nn2Cc1ncccn1]

3.72 g (14.142 mmol) of 5-fluoro-3-iodo-1H-pyrazolo[3,4-b]pyridine and 5.069 g (15.557 mmol) of caesium carbonate were initially charged in DMF (50 ml), and 2.00 g (15.557 mmol) of 2-(chloromethyl)pyrimidine dissolved in DMF (20 ml) were then added dropwise. The mixture was stirred at RT overnight. The mixture was then cooled and poured into 200 ml of water. A precipitate was formed, and this precipitate was filtered off, washed with water and dried under high vacuum overnight. This gave 2.26 g of the title compound. A precipitate then formed in the filtrate, and this precipitate was again filtered off, washed with water and dried under high vacuum overnight. This gave a further 162 mg of the title compound. In total, 2.42 g (48% of theory) of the title compound were obtained.

LC-MS (Method 4): Rₜ=1.85 min

MS (ESIpos): m/z=356 (M+H)⁺

¹H NMR (400 MHz, DMSO-d₆): δ=5.91 (s, 2H), 7.43 (t, 1H), 7.95 (dd, 1H), 8.64 (m, 1H), 8.72 (d, 2H).

### Example 7A

### 5-Fluoro-1-(pyrimidin-2-ylmethyl)-1H-pyrazolo[3,4-b]pyridine-3-carbonitrile

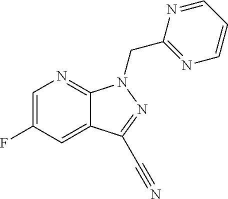 [SMILES: N#Cc1nn(Cc2ncccn2)c2ncc(F)cc12]

2.418 g (6.809 mmol) of Example 6A and 0.671 g (7.490 mmol) of copper(I) cyanide were initially charged in DMSO (40 ml) and stirred at 150° C. for 3 h. After cooling, the reaction mixture was stirred with saturated aqueous ammonium chloride solution and conc. aqueous ammonia (3:1 v/v) for 30 min and then filtered through Celite, and the filter cake was washed with ethyl acetate. The phases of the filtrate were separated and the organic phase was then washed three times with a solution of saturated aqueous ammonium chloride solution and conc. aqueous ammonia (3:1 v/v). After extraction with saturated aqueous sodium chloride solution, the organic phase was dried over sodium sulphate, filtered and concentrated under reduced pressure.

Yield: 0.89 g (51% of theory)

LC-MS (Method 4): Rₜ=0.76 min

MS (ESIpos): m/z=255 (M+H)⁺

¹H NMR (400 MHz, DMSO-d₆): δ=6.08 (s, 2H), 7.45 (t, 1H), 8.54 (dd, 1H), 8.72-8.74 (m, 2H), 8.80 (m, 1H).

### Example 8A

### 5-Fluoro-1-(pyrimidin-2-ylmethyl)-1H-pyrazolo[3,4-b]pyridine-3-carboximidamide acetate

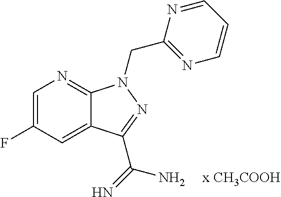

850 mg (3.343 mmol) of Example 7A in methanol (2 ml) were added to 180 mg (3.343 mmol) of sodium methoxide in methanol (5 ml), and the mixture was stirred at RT for 2 h. 214 mg (4.012 mmol) of ammonium chloride and acetic acid (0.746 ml) were then added, and the mixture was heated at reflux overnight. The mixture was then concentrated to dryness, and ethyl acetate and 1N sodium hydroxide solution were added to the residue. The phases were separated. The aqueous phase was extracted twice with ethyl acetate. The aqueous phase was concentrated and the residue was taken up in DMF. The mixture was filtered and the filter cake was washed repeatedly with DMF. The filtrate was then concentrated and dried under high vacuum overnight. This gave 514 mg (46% of theory) of the title compound.

LC-MS (Method 2): Rₜ=0.26 min

MS (ESIpos): m/z=272 (M+H)⁺

### Example 9A

### Methyl 3,3-dicyano-2,2-dimethylpropanoate

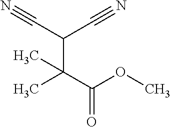

In THF (91 ml), 3 g (45.411 mmol) of malononitrile were added slowly to 1.816 g (45.411 mmol) of sodium hydride (60% in mineral oil). Subsequently, 5.876 ml (45.411 mmol) of methyl 2-bromo-2-methylpropanoate were added and the mixture was stirred at RT overnight. Another 5.876 ml (45.411 mmol) of methyl 2-bromo-2-methylpropanoate were then added and the mixture was heated at 50° C. overnight. Then yet another 1.762 ml (13.623 mmol) of methyl 2-bromo-2-methylpropanoate were added and the mixture was heated at 50° C. for a further 4 h. Saturated aqueous sodium bicarbonate solution was then added, and the reaction mixture was extracted three times with ethyl acetate. The combined organic phases were washed with saturated aqueous sodium chloride solution, dried over sodium sulphate, filtered and concentrated to dryness. This gave 8.9 g of crude product, which was purified by chromatography on silica gel (cyclohexane/ethyl acetate 4:1).

Yield: 6.47 g (85% of theory)

¹H NMR (400 MHz, DMSO-d₆): δ [ppm]=1.40 (s, 6H), 3.74 (s, 3H), 5.27 (s, 1H).

### Example 10A

### 5-Fluoro-1-(pyrimidin-2-ylmethyl)-1H-pyrazolo[3,4-b]pyridine-3-carboximidohydrazide

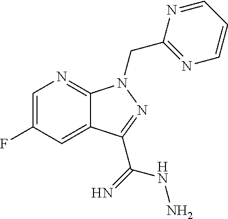 [SMILES: N=C(NN)c1nn(Cc2ncccn2)c2ncc(F)cc12]

514 mg (1.551 mmol) of the compound from Example 8A were dissolved in 10.0 ml of ethanol, and 627 mg (6.206 mmol) of triethylamine and 97 mg (1.551 mmol) of hydrazine hydrate (80% strength solution in water) were added at 0° C. The mixture was stirred at RT overnight and then concentrated on a rotary evaporator. The residue was extracted with ethyl acetate and 10% strength aqueous sodium chloride solution. The phases were separated and the aqueous phase was extracted three times with ethyl acetate. The combined organic phases were dried with sodium sulphate, filtered and concentrated to dryness. This gave 234 mg (52% of theory) of the title compound.

LC-MS (Method 4): Rₜ=0.72 min; MS (ESIpos): m/z=287 (M+H)⁺

### Example 11A

### Methyl 2-{3-[5-fluoro-1-(pyrimidin-2-ylmethyl)-1H-pyrazolo[3,4-b]pyridin-3-yl]-5-hydroxy-1,2,4-triazin-6-yl}-2-methylpropanoate

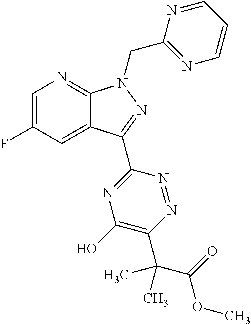 [SMILES: COC(=O)C(C)(C)c1nnc(-c2nn(Cc3ncccn3)c3ncc(F)cc23)nc1O]

229 mg (1.221 mmol) of dimethyl 2,2-dimethyl-3-oxobutanedioate (described in J. Am. Chem. Soc. 124(14), 3680-3691; 2002) were initially charged in 5 ml of ethanol and heated to reflux. 233 mg (0.814 mmol) of Example 10A, suspended in 10 ml of ethanol, were then added dropwise. The mixture was heated at reflux overnight. After cooling, the reaction mixture was concentrated and filtered off from a residue which was washed with ethanol. The filtrate was concentrated and treated with diethyl ether. A precipitate formed, which was separated off and washed with diethyl ether. This gave 164 mg of the title compound (47% of theory).

LC-MS (Method 2): Rₜ=0.76 min; MS (ESIpos): m/z=425 (M+H)⁺

¹H NMR (400 MHz, DMSO-d₆): δ [ppm]=1.44 (s, 6H), 3.56 (s, 3H), 6.09 (s, 2H), 7.45 (t, 1H), 8.44 (dd, 1H), 8.74-8.77 (m, 3H), 14.60 (s br, 1H).

### Example 12A

### 5-Fluoro-1-[(3-fluoropyridin-2-yl)methyl]-3-iodo-1H-pyrazolo[3,4-b]pyridine

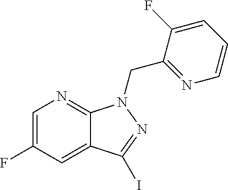 [SMILES: Fc1cnc2c(c1)c(I)nn2Cc1ncccc1F]

6.291 g (23.921 mmol) of 5-fluoro-3-iodo-1H-pyrazolo[3,4-b]pyridine and 8.573 g (26.313 mmol) of caesium carbonate were initially charged in DMF (10 ml), and 5.00 g (26.313 mmol) of 2-(bromomethyl)-3-fluoropyridine dissolved in DMF (20 ml) were then added dropwise. The mixture was stirred at RT overnight. The mixture was then cooled and poured into 200 ml of water. A precipitate was filtered off with suction, washed with water and dried under high vacuum overnight. This gave 6.28 g (70% of theory) of the title compound.

LC-MS (Method 4): Rₜ=2.17 min

MS (ESIpos): m/z=373 (M+H)⁺

¹H NMR (400 MHz, DMSO-d₆): δ=5.88 (s, 2H), 7.42-7.46 (m, 1H), 7.77 (dd, 1H), 7.93 (dd, 1H), 8.27 (d, 1H), 8.67 (t, 1H).

### Example 13A

### 5-Fluoro-1-[(3-fluoropyridin-2-yl)methyl]-1H-pyrazolo[3,4-b]pyridine-3-carbonitrile

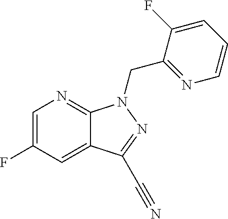 [SMILES: N#Cc1nn(Cc2ncccc2F)c2ncc(F)cc12]

6.280 g (16.876 mmol) of Example 12A and 1.663 g (18.564 mmol) of copper(I) cyanide were initially charged in DMSO (100 ml) and stirred at 150° C. for 3 h. After cooling, the reaction mixture was filtered through Celite and the filter cake was washed with ethyl acetate. The filtrate was extracted four times with saturated aqueous ammonium chloride solution and conc. aqueous ammonia (3:1 v/v), and the organic phase was separated off. The organic phase was then washed with saturated aqueous sodium chloride solution, dried over sodium sulphate, filtered and concentrated under reduced pressure. This gave 3.97 g (86% of theory) of the title compound.

LC-MS (Method 2): Rₜ=0.92 min

MS (ESIpos): m/z=272 (M+H)⁺

¹H NMR (400 MHz, DMSO-d₆): δ=6.04 (s, 2H), 7.44-7.48 (m, 1H), 7.61 (t, 1H), 8.26 (d, 1H), 8.52 (dd, 1H), 8.83 (dd, 1H).

### Example 14A

### 5-Fluoro-1-[(3-fluoropyridin-2-yl)methyl]-1H-pyrazolo[3,4-b]pyridine-3-carboximidamide acetate

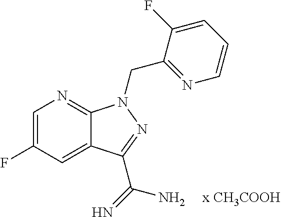

3.900 g (14.379 mmol) of Example 13A in methanol (40 ml) were added to 777 mg (14.379 mmol) of sodium methoxide in methanol (20 ml), and the mixture was stirred at RT for 2 h. 932 mg (17.255 mmol) of ammonium chloride and acetic acid (3.210 ml) were then added, and the mixture was heated at reflux overnight. The reaction mixture was then concentrated to dryness, ethyl acetate and 1N sodium hydroxide solution were added to the residue and the mixture was stirred at RT for 2 h. A solid was then filtered off and washed with ethyl acetate and water. The solid was dried under high vacuum overnight. This gave 0.56 g (11% of theory) of the title compound. The phases of the filtrate were separated, and the aqueous phase was extracted twice with ethyl acetate. The combined organic phases were dried over sodium sulphate and concentrated. This gave a further 1.86 g (14% of theory, purity 39%) of the title compound. The aqueous phase was likewise concentrated, DMF was added to the residue and the mixture was stirred at RT for 30 min A precipitate was filtered off with suction and washed with DMF, and the filtrate was concentrated and dried under high vacuum overnight. This gave a further 1.77 g (35% of theory) of the title compound.

LC-MS (Method 4): Rₜ=1.25 min

MS (ESIpos): m/z=289 (M+H)⁺

### Example 15A

### 5-Fluoro-1-[(3-fluoropyridin-2-yl)methyl]-1H-pyrazolo[3,4-b]pyridine-3-carboximidohydrazide

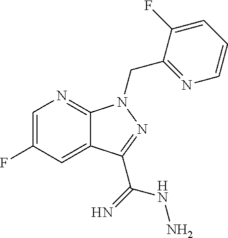 [SMILES: N=C(NN)c1nn(Cc2ncccc2F)c2ncc(F)cc12]

1.770 g (5.082 mmol) of the compound from Example 14A were dissolved in 40 ml of ethanol, and 2.057 g (20.326 mmol) of triethylamine and 317 mg (5.082 mmol) of hydrazine hydrate (80% strength solution in water) were added at 0° C. The mixture was stirred at RT overnight and then concentrated on a rotary evaporator. This gave 1.70 g of the title compound as a crude product which was reacted in the next step without further purification.

LC-MS (Method 4): Rₜ=1.23 min; MS (ESIpos): m/z=304 (M+H)⁺

### Example 16A

### Methyl 2-{3-[5-fluoro-1-(pyrimidin-2-ylmethyl)-1H-pyrazolo[3,4-b]pyridin-3-yl]-5-hydroxy-1,2,4-triazin-6-yl}-2-methylpropanoate

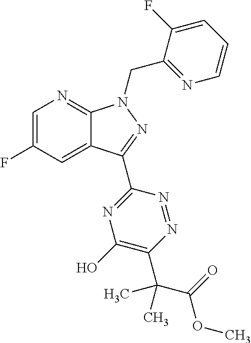 [SMILES: COC(=O)C(C)(C)c1nnc(-c2nn(Cc3ncccc3F)c3ncc(F)cc23)nc1O]

1.434 g (7.620 mmol) of dimethyl 2,2-dimethyl-3-oxobutanedioate (described in J. Am. Chem. Soc. 124(14), 3680-3691; 2002) were initially charged in 30 ml of ethanol and heated to reflux. 1.70 g of the crude compound from Example 15A, suspended in 30 ml of ethanol, were then added dropwise. The mixture was heated at reflux overnight. After cooling, the reaction mixture was concentrated and filtered off from a residue. The filtrate was concentrated and treated with diethyl ether. A precipitate formed, which was separated off and washed with diethyl ether. The filtrate was concentrated and dried under high vacuum. Diethyl ether was then added to this residue, the mixture was treated in an ultrasonic bath for 10 min and then stirred at RT for 10 min. A precipitate was filtered off, washed with a little diethyl ether and then dried under high vacuum. This gave 356 mg of the title compound (15% of theory).

LC-MS (Method 2): Rₜ=0.93 min; MS (ESIpos): m/z=442 (M+H)⁺

¹H NMR (400 MHz, DMSO-d₆): δ [ppm]=1.43 (s, 6H), 3.55 (s, 3H), 6.06 (s, 2H), 7.43-7.47 (m, 1H), 7.78-7.83 (m, 1H), 8.25 (d, 1H), 8.43 (dd, 1H), 8.78 (dd, 1H), 14.54 (br s, 1H).

### Example 17A

### (3,5-Difluoropyridin-4-yl)methyl methanesulphonate

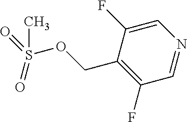

1.950 g (13.438 mmol) of (3,5-difluoropyridin-4-yl)methanol (described in WO 2010/132999) were initially charged in 50 ml of dichloromethane, and 2.341 ml (13.438 mmol) of N-ethyl-N-isopropylpropane-2-amine were added at 0° C. 1.144 ml (14.782 mmol) of methanesulphonyl chloride were then added dropwise, and the mixture was stirred at 0° C. for a further 15 min and then warmed to room temperature overnight. The mixture was then concentrated and co-distilled twice with toluene. Drying under high vacuum gave 5.39 g (about 15% of theory) of the title compound which was used without further purification for the next step.

LC-MS (Method 2): Rₜ=0.59 min; MS (ESIpos): m/z=224 (M+H)⁺

### Example 18A

### 1-[(3,5-Difluoropyridin-4-yl)methyl]-5-fluoro-3-iodo-1H-pyrazolo[3,4-b]pyridine

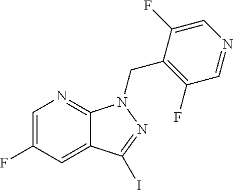 [SMILES: Fc1cnc2c(c1)c(I)nn2Cc1c(F)cncc1F]

Analogously to the procedure of Example 12A, 3.214 g (12.218 mmol) of 5-fluoro-3-iodo-1H-pyrazolo[3,4-b]pyridine were reacted with Example 17A. This gave 2.80 g (58% of theory) of the title compound.

LC-MS (Method 2): Rₜ=1.06 min

MS (ESIpos): m/z=391 (M+H)⁺

### Example 19A

### 1-[(3,5-Difluoropyridin-4-yl)methyl]-5-fluoro-1H-pyrazolo[3,4-b]pyridine-3-carbonitrile

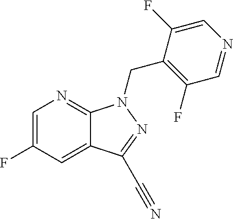 [SMILES: N#Cc1nn(Cc2c(F)cncc2F)c2ncc(F)cc12]

2.795 g (7.165 mmol) of Example 18A were reacted analogously to the procedure of Example 13A. This gave 1.95 g (93% of theory) of the title compound.

LC-MS (Method 3): Rₜ=0.95 min

MS (ESIpos): m/z=290 (M+H)⁺

### Example 20A

### 1-[(3,5-Difluoropyridin-4-yl)methyl]-5-fluoro-1H-pyrazolo[3,4-b]pyridine-3-carboximidamide acetate

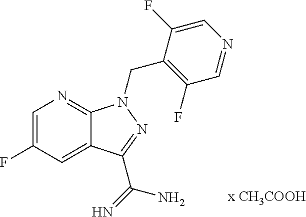 [SMILES: N=C(N)c1nn(Cc2c(F)cncc2F)c2ncc(F)cc12]

1.944 g (6.721 mmol) of Example 19A were reacted analogously to the procedure of Example 14A. This gave 1.96 g (79% of theory) of the title compound.

LC-MS (Method 3): Rₜ=0.44 min

MS (ESIpos): m/z=307 (M+H)⁺

### Example 21A

### 1-[(3,5-Difluoropyridin-4-yl)methyl]-5-fluoro-1H-pyrazolo[3,4-b]pyridine-3-carboximidohydrazide

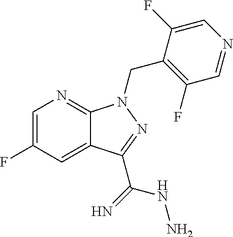 [SMILES: N=C(NN)c1nn(Cc2c(F)cncc2F)c2ncc(F)cc12]

1.360 g (3.713 mmol) of the compound from example 20A were reacted analogously to the procedure of Example 15A. This gave 1.40 g of the title compound as a crude product which was reacted in the next step without further purification.

LC-MS (Method 4): Rₜ=1.30 min; MS (ESIpos): m/z=322 (M+H)⁺

### Example 22A

### Methyl 2-(3-{1-[(3,5-difluoropyridin-4-yl)methyl]-5-fluoro-1H-pyrazolo[3,4-b]pyridin-3-yl}-5-hydroxy-1,2,4-triazin-6-yl)-2-methylpropanoate

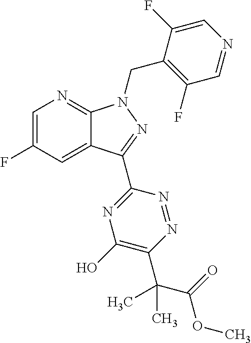 [SMILES: COC(=O)C(C)(C)c1nnc(-c2nn(Cc3c(F)cncc3F)c3ncc(F)cc23)nc1O]

1.062 g (3.306 mmol) of Example 21A were reacted analogously to the procedure of Example 16A. Purification by preparative HPLC (acetonitrile:water gradient). This gave 240 mg of the title compound (15% of theory).

LC-MS (Method 4): Rₜ=2.01 min; MS (ESIpos): m/z=460 (M+H)⁺

### Example 23A

### 4-(Chloromethyl)-3-fluoropyridine hydrochloride

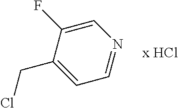

6.710 g (52.785 mmol) of (3-fluoropyridin-4-yl)methanol were initially charged in 29 ml of acetonitrile and heated to 50° C. A solution of 7.701 ml of thionyl chloride in 14.5 ml of acetonitrile was then added dropwise, and the reaction mixture was stirred at 50° C. for 4 h. The reaction mixture was then concentrated and co-distilled three times with dichloromethane. Drying under high vacuum gave 10.27 g of the title compound which was used without further purification for the next step.

### Example 24A

### 5-Fluoro-1-[(3-fluoropyridin-4-yl)methyl]-3-iodo-1H-pyrazolo[3,4-b]pyridine

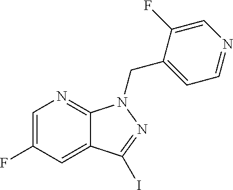 [SMILES: Fc1cnc2c(c1)c(I)nn2Cc1ccncc1F]

Analogously to the procedure of Example 12A, 12.225 g (46.482 mmol) of 5-fluoro-3-iodo-1H-pyrazolo[3,4-b]pyridine were reacted with Example 23A. This gave 11.34 g (65% of theory) of the title compound.

LC-MS (Method 3): Rₜ=1.01 min

MS (ESIpos): m/z=373 (M+H)⁺

### Example 25A

### 5-Fluoro-1-[(3-fluoropyridin-4-yl)methyl]-1H-pyrazolo[3,4-b]pyridine-3-carbonitrile

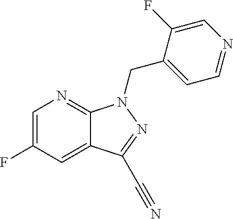 [SMILES: N#Cc1nn(Cc2ccncc2F)c2ncc(F)cc12]

11.340 g (30.474 mmol) of Example 24A were reacted analogously to the procedure of Example 13A. This gave 6.31 g (76% of theory) of the title compound.

LC-MS (Method 3): Rₜ=0.89 min

MS (ESIpos): m/z=272 (M+H)⁺

### Example 26A

### 5-Fluoro-1-[(3-fluoropyridin-4-yl)methyl]-1H-pyrazolo[3,4-b]pyridine-3-carboximidamide acetate

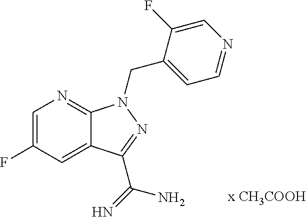 [SMILES: N=C(N)c1nn(Cc2ccncc2F)c2ncc(F)cc12]

6.310 g (23.264 mmol) of Example 25A were reacted analogously to the procedure of Example 14A. This gave 6.12 g (75% of theory) of the title compound.

LC-MS (Method 2): Rₜ=0.45 min

MS (ESIpos): m/z=289 (M+H)⁺

### Example 27A

### 5-Fluoro-1-[(3-fluoropyridin-4-yl)methyl]-1H-pyrazolo[3,4-b]pyridine-3-carboximidohydrazide

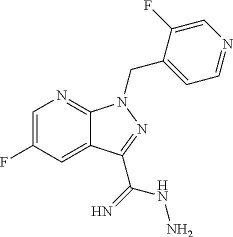 [SMILES: N=C(NN)c1nn(Cc2ccncc2F)c2ncc(F)cc12]

2.00 g (5.742 mmol) of the compound from example 26A were reacted analogously to the procedure of Example 15A. This gave 2.07 g of the title compound as a crude product which was reacted in the next step without further purification.

LC-MS (Method 2): Rₜ=0.37 min; MS (ESIpos): m/z=304 (M+H)⁺

### Example 28A

### Methyl 2-(3-{5-fluoro-1-[(3-fluoropyridin-4-yl)methyl]-1H-pyrazolo[3,4-b]pyridin-3-yl}-5-hydroxy-1,2,4-triazin-6-yl)-2-methylpropanoate

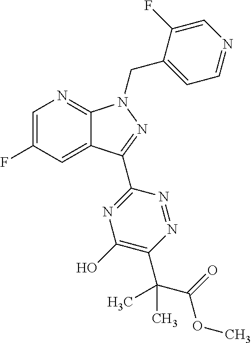 [SMILES: COC(=O)C(C)(C)c1nnc(-c2nn(Cc3ccncc3F)c3ncc(F)cc23)nc1O]

1.741 g (5.742 mmol) of Example 27A were reacted analogously to the procedure of Example 16A. Purification by preparative HPLC (acetonitrile:water gradient). This gave 740 mg of the title compound (29% of theory).

LC-MS (Method 2): Rₜ=0.84 min; MS (ESIpos): m/z=442 (M+H)⁺

### Example 29A

### 5-Fluoro-3-iodo-1-(4-methoxybenzyl)-1H-pyrazolo[3,4-b]pyridine

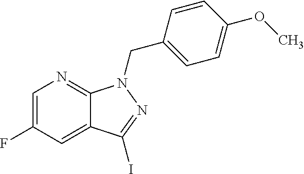 [SMILES: COc1ccc(Cn2nc(I)c3cc(F)cnc32)cc1]

10.00 g (38.021 mmol) of Example 5A were reacted analogously to the procedure of Example 6A with 4-methoxybenzyl chloride. Chromatography on silica gel (mobile phase: cyclohexane/ethyl acetate mixture) gave 8.94 g (61% of theory) of the title compound.

LC-MS (Method 3): Rₜ=1.25 min

MS (ESIpos): m/z=384 (M+H)⁺

### Example 30A

### 5-Fluoro-1-(4-methoxybenzyl)-1H-pyrazolo[3,4-b]pyridine-3-carbonitrile

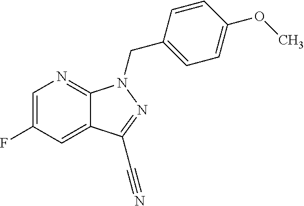 [SMILES: COc1ccc(Cn2nc(C#N)c3cc(F)cnc32)cc1]

8.94 g (23.332 mmol) of Example 29A were reacted analogously to the procedure of Example 7A. The crude product obtained in this manner was reacted without further purification.

Yield: 6.52 g (99% of theory)

LC-MS (Method 2): Rₜ=1.11 min

MS (ESIpos): m/z=283 (M+H)⁺

### Example 31A

### 5-Fluoro-1-(4-methoxybenzyl)-1H-pyrazolo[3,4-b]pyridine-3-carboximidamide acetate

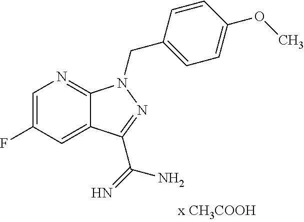 [SMILES: COc1ccc(Cn2nc(C(=N)N)c3cc(F)cnc32)cc1]

6.52 g (23.098 mmol) of Example 30A were reacted analogously to the procedure of Example 8A.

Yield: 6.16 g (74% of theory)

LC-MS (Method 3): Rₜ=0.55 min

MS (ESIpos): m/z=300 (M+H)⁺

### Example 32A

### 5-Fluoro-1-(4-methoxybenzyl)-1H-pyrazolo[3,4-b]pyridine-3-carboximidohydrazide

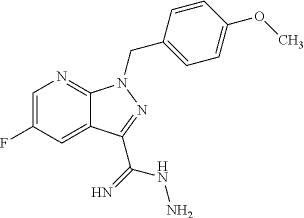 [SMILES: COc1ccc(Cn2nc(C(=N)NN)c3cc(F)cnc32)cc1]

6.16 g (17.141 mmol) of Example 31A were reacted analogously to the procedure of Example 15A. Purification on silica gel was dispensed with. This gave 4.90 g (90% of theory) of the title compound.

LC-MS (Method 3): Rₜ=0.57 min; MS (ESIpos): m/z=315 (M+H)⁺

### Example 33A

### Methyl 2-{3-[5-fluoro-1-(4-methoxybenzyl)-1H-pyrazolo[3,4-b]pyridin-3-yl]-5-hydroxy-1,2,4-triazin-6-yl}-2-methylpropanoate

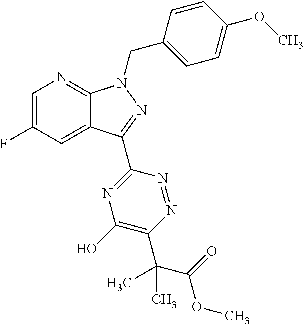 [SMILES: COC(=O)C(C)(C)c1nnc(-c2nn(Cc3ccc(OC)cc3)c3ncc(F)cc23)nc1O]

4.89 g (15.557 mmol) of the crude compound from Example 32A were reacted analogously to the procedure of Example 16A with 4.391 g (23.336 mmol) of dimethyl 2,2-dimethyl-3-oxobutanedioate (described in J. Am. Chem. Soc. 124(14), 3680-3691; 2002). After complete conversion, a solid was filtered off, washed with ethanol and then dried under high vacuum. This gave 6.04 g (85% of theory) of the title compound.

LC-MS (Method 3): Rₜ=1.05 min; MS (ESIpos): m/z=453 (M+H)⁺

### Example 34A

### 3-[5-Fluoro-1-(4-methoxybenzyl)-1H-pyrazolo[3,4-b]pyridin-3-yl]-7,7-dimethyl-5,7-dihydro-6H-pyrrolo[2,3-e][1,2,4]triazin-6-one

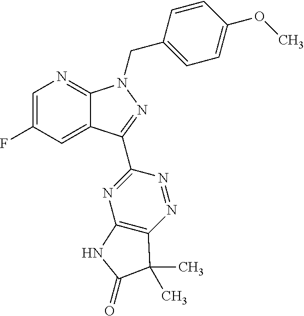 [SMILES: COc1ccc(Cn2nc(-c3nnc4c(n3)NC(=O)C4(C)C)c3cc(F)cnc32)cc1]

6.04 g (13.350 mmol) of the compound from Example 33A were reacted analogously to the procedure of Example 1. After drying under high vacuum, this gave 1.27 g (22% of theory) of the title compound.

LC-MS (Method 2): Rₜ=1.02 min; MS (EIpos): m/z=420 [M+H]⁺.

¹H NMR (400 MHz, DMSO-d₆): δ [ppm]=1.45 (s, 6H), 3.70 (s, 3H), 5.75 (s, 2H), 6.88 (d, 2H), 7.29 (d, 2H), 8.53 (dd, 1H), 8.78 (dd, 1H), 12.18 (s br, 1H).

### Example 35A

### 3-[5-Fluoro-1-(4-methoxybenzyl)-1H-pyrazolo[3,4-b]pyridin-3-yl]-7,7-dimethyl-5-{[2-(trimethylsilyl)ethoxy]methyl}-5,7-dihydro-6H-pyrrolo[2,3-e][1,2,4]triazin-6-one

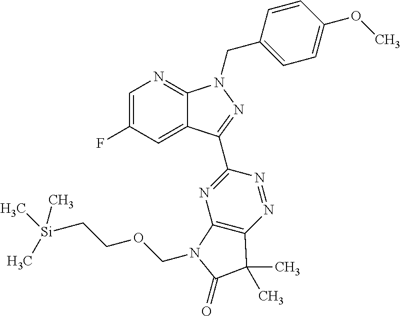

2.067 g (6.345 mmol) of caesium carbonate in DMF (30 ml) were added to 2.45 g (5.768 mmol) of the compound from Example 34A. 1.221 ml (6.922 mmol) of 2-(trimethylsilyl)ethoxymethyl chloride were then added, and the mixture was stirred at room temperature for 1 h. The solids were then filtered off and washed with DMF, the filtrate was concentrated and the residue was dried under high vacuum. This gave 4.45 g of crude material which were used without further purification for the next step.

LC-MS (Method 2): Rₜ=1.43 min; MS (EIpos): m/z=550 [M+H]⁺.

### Example 36A

### 3-(5-Fluoro-1H-pyrazolo[3,4-b]pyridin-3-yl)-7,7-dimethyl-5-{[2-(trimethylsilyl)ethoxy]methyl}-5,7-dihydro-6H-pyrrolo[2,3-e][1,2,4]triazin-6-one

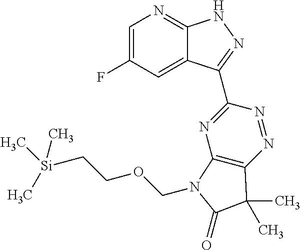

4.148 g (7.546 mmol) of the compound from Example 35A were taken up in acetonitrile (110 ml) and water (55 ml), 12.411 g (22.638 mmol) of ammonium cerium(IV) nitrate were added and the mixture was stirred at room temperature for 20 min. Plenty of water was then added, and a precipitate was filtered off. This solid was washed with water and subsequently with a little diethyl ether. This gave, after drying under high vacuum, 1.53 g (47% of theory) of the title compound.

LC-MS (Method 2): Rₜ=1.14 min; MS (EIpos): m/z=430 [M+H]⁺.

### Example 37A

### 3-{1-[(3,5-Difluoropyridin-2-yl)methyl]-5-fluoro-1H-pyrazolo[3,4-b]pyridin-3-yl}-7,7-dimethyl-5-{[2-(trimethylsilyl)ethoxy]methyl}-5,7-dihydro-6H-pyrrolo[2,3-e][1,2,4]triazin-6-one

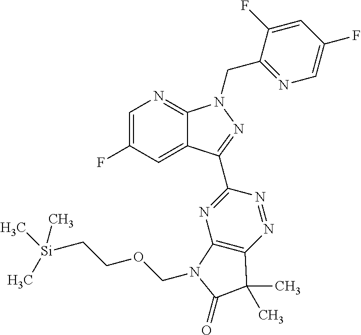 [SMILES: CC1(C)C(=O)N(COCC[Si](C)(C)C)c2nc(-c3nn(Cc4ncc(F)cc4F)c4ncc(F)cc34)nnc21]

In a flask, 137 mg (0.524 mmol) of triphenylphosphine were dissolved in 3 ml of tetrahydrofuran and 3 ml dichloromethane, and the mixture was cooled to 0° C. 101 μl (0.524 mmol) of diisopropyl azodicarboxylate were then added, and the solution was stirred at 0° C. for 1 h (solution 1). In a further flask, 0.150 g (0.349 mmol) of the compound from Example 36A and 76 mg (0.542 mmol) of (3,5-difluoropyridin-2-yl)methanol were dissolved in tetrahydrofuran (6 ml), and the mixture was cooled to 0° C. (solution 2). Solution 1 was then added to this solution 2, and the reaction mixture was stirred at room temperature for 2 h. Subsequently, once more solution 1 was prepared as described above from 274 mg (1.048 mmol) of triphenylphosphine and 203 μl (1.048 mmol) of diisopropyl azodicarboxylate and, together with 152 mg (1.048 mmol) of (3,5-difluoropyridin-2-yl)methanol, added to the reaction mixture at 0° C. After 2 h at room temperature, the product was purified by preparative HPLC (acetonitrile:water (+0.05% formic acid) gradient). This gave 64 mg of the title compound as a mixture of isomers (N1/N2-alkylated, ratio 4.5:1) (33% of theory).

LC-MS (Method 2): Rₜ=1.32 min (N2) and 1.36 min (N1); MS (EIpos): m/z=557 [M+H]⁺.

### Example 38A

### 5-Fluoro-3-iodo-1-(pyrimidin-5-ylmethyl)-1H-pyrazolo[3,4-b]pyridine

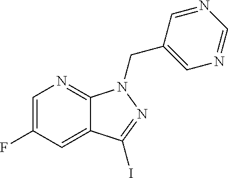 [SMILES: Fc1cnc2c(c1)c(I)nn2Cc1cncnc1]

7.479 g (28.516 mmol) of triphenylphosphine were dissolved in 120 ml of a mixture of THF/dichloromethane (v/v=1:1), and the solution was cooled to 0° C. 5.766 g (28.516 mmol) of diisopropyl azodicarboxylate (DIAD) were then added, and the solution was stirred at 0° C. for 1 h. Parallel thereto, 5.000 g (19.010 mmol) of 5-fluoro-3-iodo-1H-pyrazolo[3,4-b]pyridine and 3.140 g (28.516 mmol) of pyrimidin-5-ylmethanol (the preparation of the compound is described in J. Org. Chem. 2000, 65, 9261) were dissolved in 150 ml of a mixture of THF/dichloromethane (v/v=1:1). At 0° C., the triphenylphosphine/DIAD solution was added dropwise to this solution. The mixture was allowed to warm to RT and stirred at RT for 3 h. The reaction mixture was concentrated on a rotary evaporator and purified by means of preparative HPLC (mobile phase: water/methanol/water with 1% TFA, ratio 60:35:5). This gave 3.730 g of the target compound (purity 93%; 51% of theory).

LC-MS (Method 2): Rₜ=0.80 min; MS (ESIpos): m/z=356 (M+H)±

### Example 39A

### 5-Fluoro-1-(pyrimidin-5-ylmethyl)-1H-pyrazolo[3,4-b]pyridine-3-carbonitrile

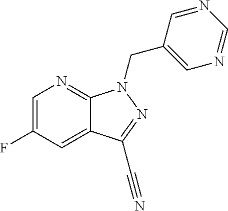 [SMILES: N#Cc1nn(Cc2cncnc2)c2ncc(F)cc12]

Under an argon atmosphere, 2.000 g (5.632 mmol) of 1,5-fluoro-3-iodo-1-(pyrimidin-5-ylmethyl)-1H-pyrazolo[3,4-b]pyridine and 555 mg (6.195 mmol) of copper(I) cyanide were initially charged in 16 ml of absolute DMSO, and the mixture was heated at 150° C. for 2 h. After cooling, the mixture was filtered through Celite and the filter cake was washed with THF and ethyl acetate. The solution was washed twice with a mixture of 25% strength ammonia solution and saturated aqueous ammonium chloride solution (v/v=1:3). The organic phase was then washed with a saturated aqueous sodium chloride solution, dried over sodium sulphate and concentrated on a rotary evaporator. The residue was taken up in ethyl acetate and filtered off with suction through silica gel, and the filter cake was washed with ethyl acetate. The organic phase was concentrated and the residue was dried under high vacuum. This gave 931 mg (65% of theory) of the target compound.

LC-MS (Method 4): Rₜ=1.63 min; MS (ESIpos): m/z=255 (M+H)⁺

### Example 40A

### 5-Fluoro-1-(pyrimidin-5-ylmethyl)-1H-pyrazolo[3,4-b]pyridine-3-carboximidamide acetate

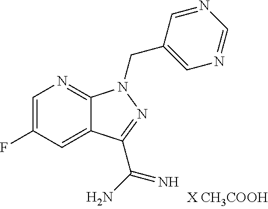 [SMILES: N=C(N)c1nn(Cc2cncnc2)c2ncc(F)cc12]

Under an argon atmosphere, 206 mg (3.664 mmol) of sodium methoxide were dissolved in 25 ml of absolute methanol, 931 mg (3.664 mmol) of 5-fluoro-1-(pyrimidin-5-ylmethyl)-1H-pyrazolo[3,4-b]pyridine-3-carbonitrile were added and the mixture was stirred at RT for 1 h. 31 mg (0.550 mmol) of sodium methoxide were added, and the mixture was stirred at RT for 15 min. 858 mg (14.288 mmol) of acetic acid and 482 mg (14.288 mmol) of ammonium chloride were then added, and the suspension was heated at reflux for 1 h. The reaction mixture was concentrated and the residue was partitioned between 1N aqueous sodium hydroxide solution and ethyl acetate. The organic phase was dried over sodium sulphate and concentrated. 902 mg (86% of theory) of the target compound were obtained.

LC-MS (Method 5): Rₜ=1.86 min; MS (ESIpos): m/z=272 (M+H)⁺

### Example 41A

### 5-Fluoro-1-(pyrimidin-5-ylmethyl)-1H-pyrazolo[3,4-b]pyridine-3-carboximidohydrazide

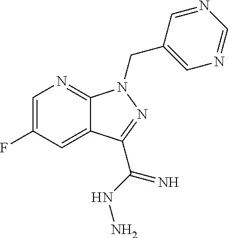 [SMILES: N=C(NN)c1nn(Cc2cncnc2)c2ncc(F)cc12]

512 mg (1.700 mmol) of 5-fluoro-1-(pyrimidin-5-ylmethyl)-1H-pyrazolo[3,4-b]pyridine-3-carboximidamide were initially charged in 15 ml of ethanol, and the mixture was cooled to 0° C. 688 mg (6.800 mmol) of triethylamine and 106 mg (1.700 mmol) of 80% hydrazine hydrate were added, and the mixture was stirred at room temperature for 18 h. The mixture was concentrated on a rotary evaporator and the residue was stirred with ethyl acetate. The filtrate was concentrated, taken up in dichloromethane and washed with 1 N aqueous sodium hydroxide solution and saturated aqueous sodium chloride solution. The organic phase was dried over sodium sulphate, concentrated on a rotary evaporator and dried under high vacuum. 88 mg (purity 92%, 17% of theory) of the target compound were obtained.

LC-MS (Method 5): Rₜ=1.78 min; MS (ESIpos): m/z=287 (M+H)⁺

### Example 42A

### Methyl 2-{3-[5-fluoro-1-(pyrimidin-5-ylmethyl)-1H-pyrazolo[3,4-b]pyridin-3-yl]-5-hydroxy-1,2,4-triazin-6-yl}-2-methylpropanoate

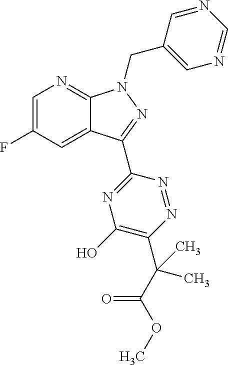 [SMILES: COC(=O)C(C)(C)c1nnc(-c2nn(Cc3cncnc3)c3ncc(F)cc23)nc1O]

455 mg (2.418 mmol) of dimethyl 2,2-dimethyl-3-oxobutanedioate were initially charged in 10 ml of ethanol, and the mixture was heated to reflux. 486 mg (1.612 mmol) of 5-fluoro-1-(pyrimidin-5-ylmethyl)-1H-pyrazolo[3,4-b]pyridine-3-carboximidohydrazide suspended in 10 ml of ethanol were then added, and the mixture was boiled under reflux overnight. After cooling, the mixture was concentrated, triturated with dichloromethane and filtered. The filtrate was purified by preparative HPLC (mobile phase: acetonitrile/water, gradient 30:70→95:5). 51 mg of the target compound were obtained (7% of theory).

LC-MS (Method 5): Rₜ=1.75 min; MS (ESIpos): m/z=425 (M+H)⁺

### Example 43A

### (3-Fluoro-2-thienyl)methanol

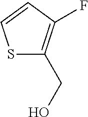

483 mg (3.016 mmol) of methyl 3-fluorothiophene-2-carboxylate were initially charged in 10 ml of tetrahydrofuran, and 3.016 ml (3.016 mmol) of lithium aluminium hydride (1 M in tetrahydrofuran) were added at 0° C. After 1 h at 0° C., the mixture was stirred at room temperature overnight. 1 ml of water, 1 ml of aqueous sodium hydroxide solution (15% strength) and 3 ml of water were then added slowly to the mixture. The mixture was then decanted from a solid and the supernatant was collected. The solid was briefly treated with tetrahydrofuran and ethyl acetate in an ultrasonic bath and then decanted. Water was added to the combined supernatants, and the phases were then separated. The organic phase was washed once more with saturated sodium chloride solution and then dried with sodium sulphate. After filtration, the mixture was concentrated under reduced pressure. This gave 417 mg of the target compound (100% of theory), which were used in the next step without further purification.

¹H NMR (400 MHz, DMSO-d₆): δ [ppm]=4.54 (dd, 2H), 5.43 (t, 1H), 6.91 (dd, 1H), 7.44 (dd, 1H).

### Example 44A

### 3-{5-Fluoro-1-[(3-fluoro-2-thienyl)methyl]-1H-pyrazolo[3,4-b]pyridin-3-yl}-7,7-dimethyl-5-{[2-(trimethylsilyl)ethoxy]methyl}-5,7-dihydro-6H-pyrrolo[2,3-e][1,2,4]triazin-6-one

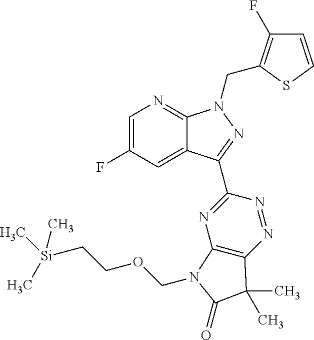 [SMILES: CC1(C)C(=O)N(COCC[Si](C)(C)C)c2nc(-c3nn(Cc4sccc4F)c4ncc(F)cc34)nnc21]

200 mg (0.466 mmol) of the compound from Example 36A were reacted analogously to the procedure of Example 37A with 184 mg (1.397 mmol) of Example 43A. After 2 h at room temperature, the product was purified by preparative HPLC (acetonitrile:water (+0.05% formic acid) gradient). This gave 58 mg of the title compound as a mixture of isomers (N1/N2-alkylated, ratio 4:1) (23% of theory).

LC-MS (Method 2): Rₜ=1.37 min (N2) and 1.42 min (N1); MS (EIpos): m/z=544 [M+H]⁺.

### WORKING EXAMPLES

### Example 1

### 3-[5-Fluoro-1-(pyrimidin-2-ylmethyl)-1H-pyrazolo[3,4-b]pyridin-3-yl]-7,7-dimethyl-5,7-dihydro-6H-pyrrolo[2,3-e][1,2,4]triazin-6-one

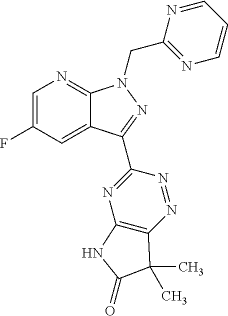 [SMILES: CC1(C)C(=O)Nc2nc(-c3nn(Cc4ncccn4)c4ncc(F)cc34)nnc21]

2.600 ml of phosphoryl chloride were added to 162 mg (0.382 mmol) of the compound from Example 11A, and the mixture was stirred at RT overnight. The reaction mixture was dissolved in 25 ml of acetonitrile and, with ice-cooling, stirred into 16 ml of concentrated aqueous ammonia solution (33% strength). The mixture was stirred at room temperature for 3 days. The reaction mixture was then concentrated. The residue was extracted with water and ethyl acetate. The phases were separated and the aqueous phase was extracted twice with ethyl acetate. The combined organic phases were washed with saturated aqueous sodium chloride solution, dried over sodium sulphate, filtered and concentrated. Acetonitrile was added to the residue. A precipitate was formed, and this precipitate was filtered off, washed with acetonitrile and then dried under high vacuum. This gave 64 mg of the title compound (43% of theory).

LC-MS (Method 3): Rₜ=0.76 min; MS (EIpos): m/z=392 [M+H]⁺.

¹H NMR (400 MHz, DMSO-d₆): δ [ppm]=1.46 (s, 6H), 6.06 (s, 2H), 7.44 (t, 1H), 8.58 (dd, 1H), 8.71-8.74 (m, 3H), 12.17 (br s, 1H).

### Example 2

### 3-{5-Fluoro-1-[(3-fluoropyridin-2-yl)methyl]-1H-pyrazolo[3,4-b]pyridin-3-yl}-7,7-dimethyl-5,7-dihydro-6H-pyrrolo[2,3-e][1,2,4]triazin-6-one

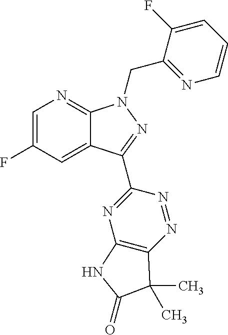 [SMILES: CC1(C)C(=O)Nc2nc(-c3nn(Cc4ncccc4F)c4ncc(F)cc34)nnc21]

5 ml of phosphoryl chloride were added to 355 mg (0.804 mmol) of the compound from Example 16A, and the mixture was stirred at RT overnight. The reaction mixture was dissolved in 53 ml of acetonitrile and, with ice-cooling, stirred into 34 ml of concentrated aqueous ammonia solution (33% strength). The mixture was stirred at room temperature for 3 days. The reaction mixture was then concentrated. Water and ethanol were added to the residue and the mixture was stirred at RT for 1 h. A precipitate was formed, and this precipitate was filtered off, washed successively with ethanol and diethyl ether and then dried under high vacuum. This gave 225 mg of the title compound (66% of theory).

LC-MS (Method 3): Rₜ=0.89 min; MS (EIpos): m/z=409 [M+H]⁺.

¹H NMR (400 MHz, DMSO-d₆): δ [ppm]=1.45 (s, 6H), 6.03 (s, 2H), 7.42-7.47 (m, 1H), 7.76-7.81 (m, 1H), 8.27 (d, 1H), 8.55 (dd, 1H), 8.75 (dd, 1H), 12.19 (br s, 1H).

### Example 3

### 4-Amino-2-{5-fluoro-1-[(3-fluoropyridin-2-yl)methyl]-1H-pyrazolo[3,4-b]pyridin-3-yl}-5,5-dimethyl-5,7-dihydro-6H-pyrrolo[2,3-d]pyrimidin-6-one

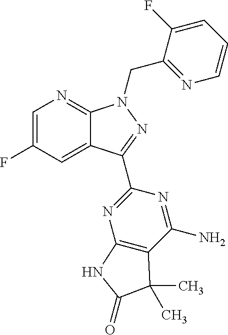 [SMILES: CC1(C)C(=O)Nc2nc(-c3nn(Cc4ncccc4F)c4ncc(F)cc34)nc(N)c21]

567 mg (1.628 mmol) of Example 14A were initially charged in tert-butanol (10 ml), and 274 mg (2.442 mmol) of potassium tert-butoxide were added. Subsequently, 324 mg (1.953 mmol) of Example 9A in tert-butanol (5 ml) were added and the mixture was heated at reflux overnight. After cooling, water and ethanol were added and the reaction mixture was stirred for 1 h. The precipitate formed was filtered off with suction and washed with a little ethanol. The solid was dried under high vacuum. This gave 568 mg of the title compound (80% of theory).

LC-MS (Method 3): Rₜ=0.82 min; MS (ESIpos): m/z=423 (M+H)⁺

¹H NMR (400 MHz, DMSO-d₆): δ [ppm]=1.34 (s, 6H), 5.94 (s, 2H), 6.87 (br s, 2H), 7.42-7.46 (m, 1H), 7.75-7.80 (m, 1H), 8.27 (d, 1H), 8.67 (dd, 1H), 8.83 (dd, 1H), 10.95 (br s, 1H).

### Example 4

### 2-{5-Fluoro-1-[(3-fluoropyridin-2-yl)methyl]-1H-pyrazolo[3,4-b]pyridin-3-yl}-4-iodo-5,5-dimethyl-5,7-dihydro-6H-pyrrolo[2,3-d]pyrimidin-6-one

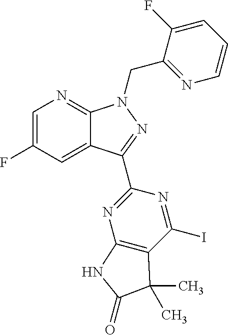 [SMILES: CC1(C)C(=O)Nc2nc(-c3nn(Cc4ncccc4F)c4ncc(F)cc34)nc(I)c21]

300 mg (0.710 mmol) of Example 3 were initially charged in isopentyl nitrite (2.03 ml) and diiodomethane (5.391 ml), and the mixture was heated to 85° C. for 1 h. After cooling, a solid was filtered off and washed with a little acetonitrile. The solid was then purified by means of preparative HPLC (acetonitrile:water (+0.05% formic acid) gradient). This gave 58 mg of the title compound (15% of theory).

LC-MS (Method 4): Rₜ=2.38 min; MS (ESIpos): m/z=534 (M+H)⁺

¹H NMR (400 MHz, DMSO-d₆): δ [ppm]=1.42 (s, 6H), 6.02 (s, 2H), 7.42-7.46 (m, 1H), 7.76-7.81 (m, 1H), 8.26 (d, 1H), 8.48 (dd, 1H), 8.73 (dd, 1H), 11.75 (s, 1H).

In addition to the title compound, 25 mg (8% of theory) of 2-[5-fluoro-1-[(3-fluoropyridin-2-yl)methyl]-1H-pyrazolo[3,4-b]pyridin-3-yl]-4-hydroxy-5,5-dimethyl-5,7-dihydro-6H-pyrrolo[2,3-d]pyrimidin-6-one (Example 5) were obtained.

### Example 5

### 2-{5-Fluoro-1-[(3-fluoropyridin-2-yl)methyl]-1H-pyrazolo[3,4-b]pyridin-3-yl}-4-hydroxy-5,5-dimethyl-5,7-dihydro-6H-pyrrolo[2,3-d]pyrimidin-6-one

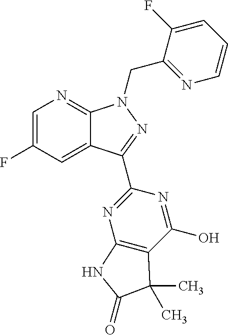 [SMILES: CC1(C)C(=O)Nc2nc(-c3nn(Cc4ncccc4F)c4ncc(F)cc34)nc(O)c21]

The title compound was formed as a byproduct in the procedure of Example 4. 25 mg (8% of theory) were obtained.

LC-MS (Method 2): Rₜ=0.83 min; MS (ESIpos): m/z=424 (M+H)⁺

¹H NMR (400 MHz, DMSO-d₆): δ[ppm]=1.32 (s, 6H), 6.00 (s, 2H), 7.43-7.47 (m, 1H), 7.77-7.81 (m, 1H), 8.26 (d, 1H), 8.57 (br s, 1H), 8.75 (s, 1H), 11.09 (br s, 1H), 12.51 (br s, 1H).

### Example 6

### 2-{5-Fluoro-1-[(3-fluoropyridin-2-yl)methyl]-1H-pyrazolo[3,4-b]pyridin-3-yl}-5,5-dimethyl-5,7-dihydro-6H-pyrrolo[2,3-d]pyrimidin-6-one

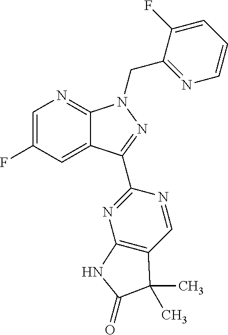 [SMILES: CC1(C)C(=O)Nc2nc(-c3nn(Cc4ncccc4F)c4ncc(F)cc34)ncc21]

53 mg (0.099 mmol) of Example 4 were dissolved in DMF (5 ml) and added to 23.88 mg of palladium on carbon (10%) in DMF (1 ml), and the mixture was hydrogenated at standard hydrogen pressure for 12 h. The mixture was then filtered through Celite, the filter cake was washed with DMF and the filtrate was concentrated to dryness. The residue was purified by preparative HPLC (acetonitrile:water (+0.05% formic acid) gradient). This gave 29 mg of the title compound (71% of theory).

LC-MS (Method 2): Rₜ=0.94 min; MS (ESIpos): m/z=408 (M+H)⁺

¹H NMR (400 MHz, DMSO-d₆): δ [ppm]=1.38 (s, 6H), 5.99 (s, 2H), 7.42-7.47 (m, 1H), 7.76-7.81 (m, 1H), 8.27 (d, 1H), 8.59 (dd, 1H), 8.63 (s, 1H), 8.71 (dd, 1H), 11.56 (br s, 1H).

### Example 7

### 4-Amino-2-{1-[(3,5-difluoropyridin-4-yl)methyl]-5-fluoro-1H-pyrazolo[3,4-b]pyridin-3-yl}-5,5-dimethyl-5,7-dihydro-6H-pyrrolo[2,3-d]pyrimidin-6-one

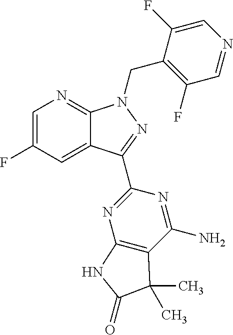 [SMILES: CC1(C)C(=O)Nc2nc(-c3nn(Cc4c(F)cncc4F)c4ncc(F)cc34)nc(N)c21]

600 mg (1.638 mmol) of Example 20A were reacted analogously to the procedure of Example 3. This gave 435 mg of the title compound in a purity of about 59%. Some of this was purified by preparative HPLC (acetonitrile:water (+0.05% formic acid) gradient). This gave 40 mg of the title compound (5% of theory).

LC-MS (Method 2): Rₜ=0.81 min; MS (ESIpos): m/z=441 (M+H)⁺

¹H NMR (400 MHz, DMSO-d₆): δ [ppm]=1.33 (s, 6H), 5.91 (s, 2H), 6.88 (br s, 2H), 8.58 (s, 2H), 8.72 (m, 1H), 8.86 (dd, 1H), 10.99 (br s, 1H).

### Example 8

### 2-{1-[(3,5-Difluoropyridin-4-yl)methyl]-5-fluoro-1H-pyrazolo[3,4-b]pyridin-3-yl}-4-iodo-5,5-dimethyl-5,7-dihydro-6H-pyrrolo[2,3-d]pyrimidin-6-one

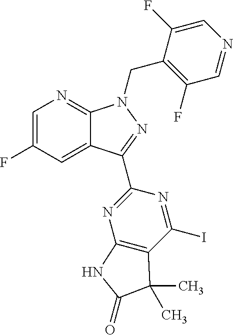 [SMILES: CC1(C)C(=O)Nc2nc(-c3nn(Cc4c(F)cncc4F)c4ncc(F)cc34)nc(I)c21]

434 mg (0.985 mmol) of Example 7 were reacted analogously to the procedure of Example 4. This gave 123 mg of the title compound (22% of theory).

LC-MS (Method 3): Rₜ=1.15 min; MS (ESIpos): m/z=552 (M+H)⁺

¹H NMR (400 MHz, DMSO-d₆): δ [ppm]=1.41 (s, 6H), 5.97 (s, 2H), 8.47 (dd, 1H), 8.58 (s, 2H), 8.79 (dd, 1H), 11.77 (s, 1H).

In addition to the title compound, 13 mg (3% of theory) of 2-{1-[(3,5-difluoropyridin-4-yl)methyl]-5-fluoro-1H-pyrazolo[3,4-b]pyridin-3-yl}-4-hydroxy-5,5-dimethyl-5,7-dihydro-6H-pyrrolo[2,3-d]pyrimidin-6-one (Example 9) were also obtained.

### Example 9

### 2-{1-[(3,5-Difluoropyridin-4-yl)methyl]-5-fluoro-1H-pyrazolo[3,4-b]pyridin-3-yl}-4-hydroxy-5,5-dimethyl-5,7-dihydro-6H-pyrrolo[2,3-d]pyrimidin-6-one

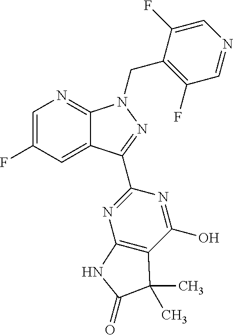 [SMILES: CC1(C)C(=O)Nc2nc(-c3nn(Cc4c(F)cncc4F)c4ncc(F)cc34)nc(O)c21]

The title compound was formed as a byproduct in the procedure of Example 8. 13 mg (3% of theory) were obtained.

LC-MS (Method 2): Rₜ=0.83 min; MS (ESIpos): m/z=442 (M+H)⁺

¹H NMR (400 MHz, DMSO-d₆): δ [ppm]=1.32 (s, 6H), 5.94 (s, 2H), 8.58 (s br, 3H), 8.81 (s br, 1H), 11.10 (s br, 1H), 12.53 (s br, 1H).

### Example 10

### 2-[1-[(3,5-Difluoropyridin-4-yl)methyl]-5-fluoro-1H-pyrazolo[3,4-b]pyridin-3-yl]-5,5-dimethyl-5,7-dihydro-6H-pyrrolo[2,3-d]pyrimidin-6-one

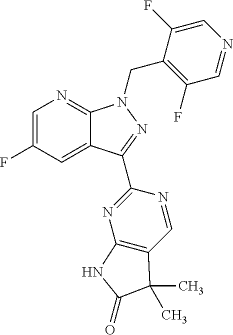 [SMILES: CC1(C)C(=O)Nc2nc(-c3nn(Cc4c(F)cncc4F)c4ncc(F)cc34)ncc21]

122 mg (0.221 mmol) of Example 8 were hydrogenated analogously to the procedure of Example 6. This gave 72 mg of the title compound (76% of theory).

LC-MS (Method 2): Rₜ=0.96 min; MS (ESIpos): m/z=426 (M+H)⁺

¹H NMR (400 MHz, DMSO-d₆): δ [ppm]=1.37 (s, 6H), 5.95 (s, 2H), 8.58-8.62 (m, 3H), 8.62 (s br, 1H), 8.77 (s br, 1H), 11.59 (br s, 1H).

### Example 11

### 3-{1-[(3,5-Difluoropyridin-4-yl)methyl]-5-fluoro-1H-pyrazolo[3,4-b]pyridin-3-yl}-7,7-dimethyl-5,7-dihydro-6H-pyrrolo[2,3-e][1,2,4]triazin-6-one

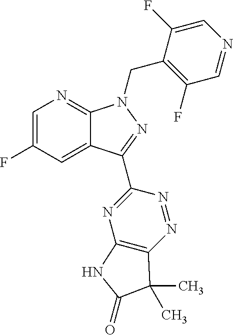 [SMILES: CC1(C)C(=O)Nc2nc(-c3nn(Cc4c(F)cncc4F)c4ncc(F)cc34)nnc21]

240 mg (0.522 mmol) of the compound from Example 22A were reacted analogously to the procedure of Example 2. This gave 181 mg of the title compound (80% of theory).

LC-MS (Method 2): Rₜ=0.91 min; MS (EIpos): m/z=427 [M+H]⁺.

¹H NMR (400 MHz, DMSO-d₆): δ [ppm]=1.44 (s, 6H), 5.98 (s, 2H), 8.55-8.59 (m, 3H), 8.81 (m, 1H), 12.19 (br s, 1H).

### Example 12

### 4-Amino-2-[5-fluoro-1-[(3-fluoropyridin-4-yl)methyl]-1H-pyrazolo[3,4-b]pyridin-3-yl]-5,5-dimethyl-5,7-dihydro-6H-pyrrolo[2,3-d]pyrimidin-6-one

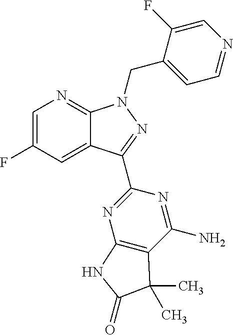 [SMILES: CC1(C)C(=O)Nc2nc(-c3nn(Cc4ccncc4F)c4ncc(F)cc34)nc(N)c21]

3.050 g (8.756 mmol) of Example 26A were reacted analogously to the procedure of Example 3. Purification by preparative chromatography on silica gel (dichloromethane:methanol gradient). This gave 528 mg of the title compound (14% of theory).

LC-MS (Method 2): Rₜ=0.80 min; MS (ESIpos): m/z=423 (M+H)⁺

¹H NMR (400 MHz, DMSO-d₆): δ [ppm]=1.35 (s, 6H), 5.90 (s, 2H), 6.89 (br s, 2H), 7.11 (t, 1H), 8.35 (d, 1H), 8.59 (d, 1H), 8.70 (dd, 1H), 8.87 (dd, 1H), 10.99 (br s, 1H).

### Example 13

### 2-{5-Fluoro-1-[(3-fluoropyridin-4-yl)methyl]-1H-pyrazolo[3,4-b]pyridin-3-yl}-4-iodo-5,5-dimethyl-5,7-dihydro-6H-pyrrolo[2,3-d]pyrimidin-6-one

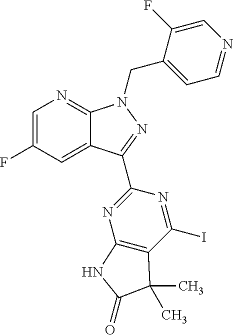 [SMILES: CC1(C)C(=O)Nc2nc(-c3nn(Cc4ccncc4F)c4ncc(F)cc34)nc(I)c21]

576 mg (1.364 mmol) of Example 12 were reacted analogously to the procedure of Example 4. This gave 74 mg of the title compound (10% of theory).

LC-MS (Method 3): Rₜ=1.10 min; MS (ESIpos): m/z=534 (M+H)⁺

¹H NMR (400 MHz, DMSO-d₆): δ [ppm]=1.43 (s, 6H), 5.97 (s, 2H), 7.12 (t, 1H), 8.36 (d, 1H), 8.50 (dd, 1H), 8.60 (m, 1H), 8.78 (m, 1H), 11.77 (s, 1H).

### Example 14

### 2-{5-Fluoro-1-[(3-fluoropyridin-4-yl)methyl]-1H-pyrazolo[3,4-b]pyridin-3-yl}-5,5-dimethyl-5,7-dihydro-6H-pyrrolo[2,3-d]pyrimidin-6-one

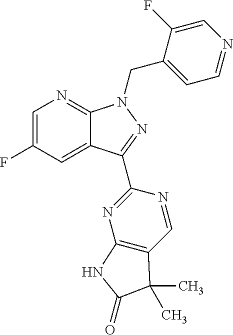 [SMILES: CC1(C)C(=O)Nc2nc(-c3nn(Cc4ccncc4F)c4ncc(F)cc34)ncc21]

130 mg (0.244 mmol) of Example 13 were hydrogenated analogously to the procedure of Example 6. This gave 54 mg of the title compound (55% of theory).

LC-MS (Method 4): Rₜ=1.99 min; MS (ESIpos): m/z=408 (M+H)⁺

¹H NMR (400 MHz, DMSO-d₆): δ [ppm]=1.38 (s, 6H), 5.94 (s, 2H), 7.16 (t, 1H), 8.37 (d, 1H), 8.60-8.63 (m, 2H), 8.65 (s, 1H), 8.75 (dd, 1H), 11.60 (s, 1H).

### Example 15

### 3-{5-Fluoro-1-[(3-fluoropyridin-4-yl)methyl]-1H-pyrazolo[3,4-b]pyridin-3-yl}-7,7-dimethyl-5,7-dihydro-6H-pyrrolo[2,3-e][1,2,4]triazin-6-one

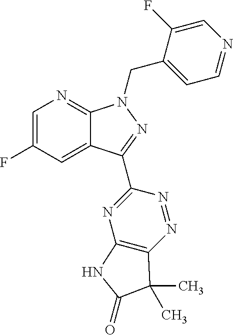 [SMILES: CC1(C)C(=O)Nc2nc(-c3nn(Cc4ccncc4F)c4ncc(F)cc34)nnc21]

772 mg (1.680 mmol) of the compound from Example 28A were reacted analogously to the procedure of Example 2. This gave 370 mg of the title compound (53% of theory).

LC-MS (Method 4): Rₜ=1.92 min; MS (EIpos): m/z=409 [M+H]⁺.

¹H NMR (400 MHz, DMSO-d₆): δ [ppm]=1.45 (s, 6H), 5.98 (s, 2H), 7.18 (t, 1H), 8.37 (d, 1H), 8.57-8.60 (m, 2H), 8.79 (m, 1H), 12.17 (br s, 1H).

### Example 16

### 3-{1-[(3,5-Difluoropyridin-2-yl)methyl]-5-fluoro-1H-pyrazolo[3,4-b]pyridin-3-yl}-7,7-dimethyl-5,7-dihydro-6H-pyrrolo[2,3-e][1,2,4]triazin-6-one

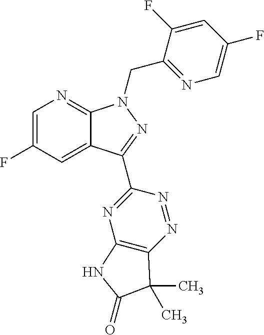 [SMILES: CC1(C)C(=O)Nc2nc(-c3nn(Cc4ncc(F)cc4F)c4ncc(F)cc34)nnc21]

63 mg (0.114 mmol) of the compound from Example 37A were stirred in dichloromethane (4 ml) and trifluoroacetic acid (1 ml) at room temperature for 3 h. The mixture was then concentrated to dryness. The residue was stirred in ethanol/2N hydrochloric acid (4:1, 10 ml) at 45° C. for 3 h. This was followed by concentration to dryness. The residue obtained was purified by means of preparative HPLC (methanol:water gradient). Purification by preparative HPLC (methanol:water gradient) gave 21 mg of the title compound (44% of theory).

LC-MS (Method 2): Rₜ=0.96 min; MS (EIpos): m/z=427 [M+H]⁺.

¹H NMR (400 MHz, DMSO-d₆): δ [ppm]=1.45 (s, 6H), 6.01 (s, 2H), 8.00-8.05 (m, 1H), 8.38 (d, 1H), 8.55 (dd, 1H), 8.75 (dd, 1H), 12.16 (s br, 1H).

### Example 17

### 2-[5-Fluoro-1-(pyrimidin-5-ylmethyl)-1H-pyrazolo[3,4-b]pyridin-3-yl]-4-iodo-5,5-dimethyl-5,7-dihydro-6H-pyrrolo[2,3-d]pyrimidin-6-one

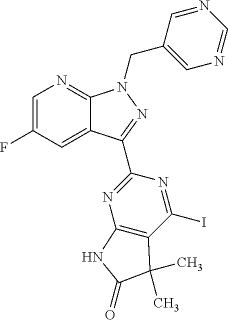 [SMILES: CC1(C)C(=O)Nc2nc(-c3nn(Cc4cncnc4)c4ncc(F)cc34)nc(I)c21]

35.234 g (131.551 mmol) of diiodomethane and 4.1 ml (30.449 mmol) of isopentyl nitrite were added to 634 mg (1.563 mmol) of 4-amino-2-[5-fluoro-1-(pyrimidin-5-ylmethyl)-1H-pyrazolo[3,4-b]pyridin-3-yl]-5,5-dimethyl-5,7-dihydro-6H-pyrrolo[2,3-d]pyrimidin-6-one. The mixture was stirred at 85° C. for 8 h. After cooling, the mixture was filtered and the filtrate was diluted with cyclohexane and sucked through silica gel. The silica gel was washed with cyclohexane and the product was eluted with dichloromethane/methanol (v/v=100:3). The collected fractions were concentrated on a rotary evaporator, and the residue was dried under high vacuum. This gave 410 mg (purity 68%, 34% of theory) of the target compound.

LC-MS (Method 3) Rₜ=0.99 min; MS (ESIpos): m/z=517 (M+H)⁺

### Example 18

### 2-[5-Fluoro-1-(pyrimidin-5-ylmethyl)-1H-pyrazolo[3,4-b]pyridin-3-yl]-5,5-dimethyl-5,7-dihydro-6H-pyrrolo[2,3-d]pyrimidin-6-one

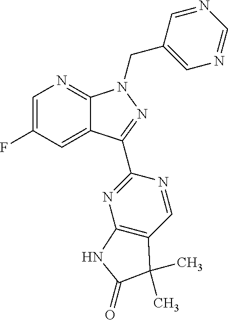 [SMILES: CC1(C)C(=O)Nc2nc(-c3nn(Cc4cncnc4)c4ncc(F)cc34)ncc21]

563 mg (purity 65%, 0.709 mmol) of 2-[5-fluoro-1-(pyrimidin-5-ylmethyl)-1H-pyrazolo[3,4-b]pyridin-3-yl]-4-iodo-5,5-dimethyl-5,7-dihydro-6H-pyrrolo[2,3-d]pyrimidin-6-one were dissolved in 20 ml of absolute DMF, 150 mg of 10% palladium on carbon were added and the mixture was hydrogenated at standard hydrogen pressure overnight. The reaction mixture was filtered through Celite and concentrated. The residue was purified by preparative HPLC (mobile phase: acetonitrile/water, gradient 30:70→95:5). This gave 47 mg (purity 92%, 16% of theory) of the target compound.

LC-MS (Method 3) Rₜ=0.78 min; MS (ESIpos): m/z=391 (M+H)⁺

¹H NMR (400 MHz, DMSO-d₆): δ [ppm], 1.38 (s, 6H), 5.90 (s, 2H), 8.60 (dd, 1H), 8.64 (s, 1H), 8.77 (dd, 1H), 8.64 (s, 2H), 9.15 (s, 1H).

### Example 19

### 3-[5-Fluoro-1-(pyrimidin-5-ylmethyl)-1H-pyrazolo[3,4-b]pyridin-3-yl]-7,7-dimethyl-5,7-dihydro-6H-pyrrolo[2,3-e][1,2,4]triazin-6-one

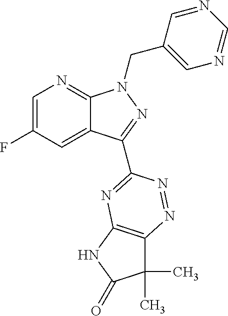 [SMILES: CC1(C)C(=O)Nc2nc(-c3nn(Cc4cncnc4)c4ncc(F)cc34)nnc21]

5 ml (53.642 mmol) of phosphoryl chloride were added to 305 mg (purity 62%, 0.445 mmol) of methyl 2-{3-[5-fluoro-1-(pyrimidin-5-ylmethyl)-1H-pyrazolo[3,4-b]pyridin-3-yl]-5-hydroxy-1,2,4-triazin-6-yl}-2-methylpropanoate, and the mixture was stirred at RT overnight. The reaction solution was diluted with 20 ml of dry acetonitrile and gradually added dropwise while cooling with ice to 112 ml of a 25% aqueous ammonia solution, and the mixture was stirred at RT overnight. The reaction mixture was concentrated on a rotary evaporator until the acetonitrile had evaporated, and the precipitate was filtered off and dried under high vacuum. This gave 194 mg of the target compound (97% of theory).

LC-MS (Method 2) Rₜ=0.79 min; MS (ESIpos): m/z=392 (M+H)⁺

¹H NMR (400 MHz, DMSO-d₆): δ [ppm], 1.30 (s, 6H), 5.91 (s, 2H), 8.56 (dd, 1H), 8.76 (dd, 1H), 8.84 (s, 2H), 9.14 (s, 1H).

### Example 20

### 3-{5-Fluoro-1-[(3-fluoro-2-thienyl)methyl]-1H-pyrazolo[3,4-b]pyridin-3-yl}-7,7-dimethyl-5,7-dihydro-6H-pyrrolo[2,3-e][1,2,4]triazin-6-one

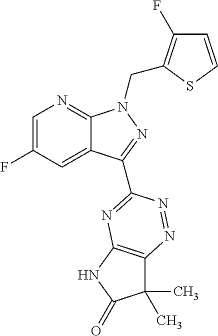 [SMILES: CC1(C)C(=O)Nc2nc(-c3nn(Cc4sccc4F)c4ncc(F)cc34)nnc21]

57.7 mg (0.106 mmol) of the compound from Example 44A were reacted analogously to the procedure of Example 16. Purification by preparative HPLC (acetonitrile: water: water with 1% trifluoroacetic acid −50:40:10) gave 16 mg of the title compound (37% of theory).

LC-MS (Method 2): Rₜ=1.02 min; MS (EIpos): m/z=414 [M+H]⁺.

¹H NMR (400 MHz, DMSO-d₆): δ [ppm]=1.46 (s, 6H), 5.93 (s, 2H), 6.98 (d, 1H), 7.50 (t, 1H), 8.54 (dd, 1H), 8.81 (s br, 1H), 12.20 (s br, 1H).

### Example 21

### 4-Amino-2-[5-fluoro-1-(pyrimidin-5-ylmethyl)-1H-pyrazolo[3,4-b]pyridin-3-yl]-5,5-dimethyl-5,7-dihydro-6H-pyrrolo[2,3-d]pyrimidin-6-one

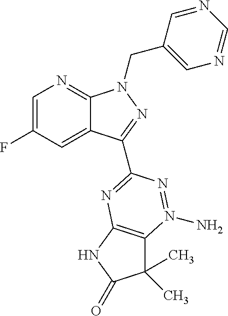

6 ml of tert-butanol, 582 mg (2.803 mmol) of methyl 3,3-dicyano-2,2-dimethylpropanoate dissolved in 3 ml of tert-butanol and 377 mg (3.363 mmol) of potassium tert-butoxide were added to 802 mg (2.803 mmol) of 5-fluoro-1-(pyrimidin-5-ylmethyl)-1H-pyrazolo[3,4-b]pyridin-3-carboximidamide, and the mixture was heated under reflux for 2 h. After cooling, the reaction mixture was filtered and the filtrate was concentrated on a rotary evaporator. The residue was triturated with water/acetonitrile and then with dichloromethane, filtered off with suction and dried under high vacuum. 263 mg (23% of theory) of the target compound were obtained.

LC-MS (Method 4) Rₜ=1.65 min; MS (ESIpos): m/z=406 (M+H)⁺

### B. ASSESSMENT OF PHARMACOLOGICAL EFFICACY

The pharmacological effect of the compounds according to the invention can be shown in the following assays:

### B-1. Vasorelaxant Effect In Vitro

Rabbits are stunned by a blow to the neck and exsanguinated. The aorta is removed, freed from adhering tissue and divided into rings of a width of 1.5 mm. The rings are placed individually under an initial tension in 5 ml organ baths with Krebs-Henseleit solution which is at 37° C., is gassed with carbogen and has the following composition (in each case mM): sodium chloride: 119; potassium chloride: 4.8; calcium chloride dihydrate: 1; magnesium sulphate heptahydrate: 1.4; potassium dihydrogenphosphate: 1.2; sodium bicarbonate: 25; glucose: 10. The contractile force is determined with Statham UC2 cells, amplified and digitalized using A/D transducers (DAS-1802 HC, Keithley Instruments Munich), and recorded in parallel on linear recorders. To produce a contraction, phenylephrine is added to the bath cumulatively in increasing concentration. After several control cycles, the substance to be investigated is added in each further run in increasing dosage in each case, and the height of the contraction achieved is compared with the height of the contraction reached in the last preceding run. This is used to calculate the concentration needed to reduce the magnitude of the control value by 50% (IC₅₀ value). The standard administration volume is 5 μl; the DMSO content in the bath solution corresponds to 0.1%.

Representative IC₅₀ values for the compounds according to the invention are shown in the table below (Table 1):

TABLE 1

| Example No. | IC₅₀ [nM] |
|---|---|
| 1 | 1320 |
| 2 | 234 |
| 3 | 34 |
| 5 | 627 |
| 6 | 82 |
| 7 | 116 |
| 10 | 259 |
| 11 | 2540 |
| 12 | 121 |
| 14 | 207 |
| 15 | 2230 |
| 18 | 196 |
| 19 | 361 |
| 21 | 99 |

### B-2. Effect on a Recombinant Guanylate Cyclase Reporter Cell Line

The cellular activity of the compounds according to the invention is determined using a recombinant guanylate cyclase reporter cell line, as described in F. Wunder et al., Anal. Biochem. 339, 104-112 (2005).

Representative values (MEC=minimum effective concentration) for the compounds according to the invention are shown in the table below (Table 2):

TABLE 2

| Example No. | MEC [μM] |
|---|---|
| 1 | 10 |
| 2 | 1 |
| 3 | 0.3 |
| 4 | 0.3 |
| 5 | 3 |
| 6 | 0.03 |
| 7 | 0.3 |
| 9 | 3.0 |
| 10 | 1.0 |
| 11 | 3.0 |
| 12 | 1.0 |
| 14 | 1.0 |
| 15 | 3.0 |
| 16 | 1.0 |
| 18 | 0.1 |
| 19 | 0.1 |
| 20 | 0.01 |
| 21 | 0.1 |

### B-3. Radiotelemetric Measurement of Blood Pressure on Conscious Spontaneously Hypertensive Rats

A commercially available telemetry system from DATA SCIENCES INTERNATIONAL DSI, USA, is employed for the blood pressure measurement on conscious rats described below.

The system consists of 3 main components:
implantable transmitters (Physiotel® telemetry transmitter)
receivers (Physiotel® receiver) which are linked via a multiplexer (DSI Data Exchange Matrix) to a
data acquisition computer.

The telemetry system makes it possible to continuously record blood pressure, heart rate and body motion of conscious animals in their usual habitat.

### Animal Material

The investigations are carried out on adult female spontaneously hypertensive rats (SHR Okamoto) with a body weight of >200 g. SHR/NCrl from the Okamoto Kyoto School of Medicine, 1963 were a cross of male Wistar Kyoto rats with highly elevated blood pressure and female rats having a slightly elevated blood pressure and at F13 handed over to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6.00 am and at 7.00 pm.

### Transmitter Implantation

The telemetry transmitters TA11 PA-C40 used are surgically implanted under aseptic conditions in the experimental animals at least 14 days before the first experimental use. The animals instrumented in this way can be employed repeatedly after the wound has healed and the implant has settled.

For the implantation, the fasted animals are anaesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and layered closure of the wound is performed.

An antibiotic (Tardomyocel COMP, Bayer, 1 ml/kg s.c.) is administered postoperatively for prophylaxis of infection.

### Substances and Solutions

Unless indicated otherwise, the substances to be investigated are administered orally by gavage in each case to a group of animals (n=6). The test substances are dissolved in suitable solvent mixtures, or suspended in 0.5% strength Tylose, appropriate for an administration volume of 5 ml/kg of body weight.

A solvent-treated group of animals is employed as control.

### Test Procedure

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch and are switched to transmission in the run-up to the experiment. The signals emitted can be detected online by a data acquisition system (Dataquest™ A.R.T. for WINDOWS, DSI) and processed accordingly. The data are stored in each case in a file created for this purpose and bearing the experiment number.

In the standard procedure, the following are measured for 10-second periods in each case:
systolic blood pressure (SBP)
diastolic blood pressure (DBP)
mean arterial pressure (MAP)
heart rate (HR)
activity (ACT).

The acquisition of measurements is repeated under computer control at 5-minute intervals. The source data obtained as absolute value are corrected in the diagram with the currently measured barometric pressure (Ambient Pressure Reference Monitor; APR-1) and stored as individual data. Further technical details are given in the extensive documentation from the manufacturing company (DSI).

Unless indicated otherwise, the test substances are administered at 9.00 am on the day of the experiment. Following the administration, the parameters described above are measured over 24 hours.

### Evaluation

After the end of the experiment, the acquired individual data are sorted using the analysis software (DATAQUEST™ A.R.T.™ ANALYSIS). The blank value is assumed to be the time 2 hours before administration, and so the selected data set encompasses the period from 7.00 am on the day of the experiment to 9.00 am the following day.

The data are smoothed over a presettable time by determination of the average (15-minute average) and transferred as a text file to a storage medium. The measured values presorted and compressed in this way are transferred into Excel templates and tabulated. For each day of the experiment, the data obtained are stored in a dedicated file bearing the number of the experiment. Results and test protocols are filed in paper form sorted by numbers.

### REFERENCES

Klaus Witte, Kai Hu, Johanna Swiatek, Claudia Müssig, Georg Ertl and Bjorn Lemmer: Experimental heart failure in rats: effects on cardiovascular circadian rhythms and on myocardial 3-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994

### B-4. Determination of Pharmacokinetic Parameters Following Intravenous and Oral Administration

The pharmacokinetic parameters of the compounds of the formula (I) according to the invention are determined in male CD-1 mice, male Wistar rats and/or female beagles. The administration volume is 5 ml/kg for mice, 5 ml/kg for rats and 0.5 ml/kg for dogs. Intravenous administration is via a formulation of species-specific plasma/DMSO (99/1) in the case of mice and rats and via water/PEG400/ethanol (50/40/10 or 30/60/10) in the case of dogs. The removal of blood from rats is simplified by inserting a silicone catheter into the right Vena jugularis externa prior to substance administration. The surgical intervention takes place one day prior to the experiment with isofluran anesthesia and administration of an analgetic (atropine/rimadyl (3/1) 0.1 ml s.c.). Substance administration is as i.v. bolus in the case of mice, as i.v. bolus or via a 15-minute infusion in the case of rats and via a 15-minute infusion in the case of dogs. Removal of blood is after 0.033, 0.083, 0.17, 0.5, 1, 2, 3, 4, 6, 7 and 24 hours in the case of mice and, after a 15-minute infusion, after 0.083, 0.25, 0.28, 0.33, 0.42, 0.75, 1, 2, 3, 4, 6, 7 (or 8 in the case of Example 3) and 24 hours in the case of dogs and rats and after an i.v. bolus administration, after 0.033, 0.083, 0.17, 0.5, 1, 2, 3, 4, 6, 7 and 24 hours in the case of rats. For all species, oral administration of the dissolved substance via gavage is carried out based on a water/PEG400/ethanol formulation (50/40/10). Here, the removal of blood from rats and dogs is after 0.083, 0.17, 0.5, 0.75, 1, 2, 3, 4, 6, 7 and 24 hours. The blood is removed into heparinized tubes. The blood plasma is then obtained by centrifugation; if required, it can be stored at −20° C. until further processing.

An internal standard (ZK 228859) is added to the samples of the compounds of the formula (I) according to the invention, calibration samples and QCs, and the protein is precipitated using excess acetonitrile. After addition of an ammonium acetate buffer (0.01 M, pH 6.8) and subsequent vortexing, the mixture is centrifuged at 1000 g and the supernatant is examined by LC-MS/MS (API 4000, AB Sciex). Chromatographic separation is carried out on an Agilent 1100-HPLC. The injection volume is 10 μl. The separation column used is a Phenomenex Luna 5μ C8(2) 100A 50×2 mm, adjusted to a temperature of 40° C. A binary mobile phase gradient at 500 μl/min is used (A: 0.01M ammonium acetate buffer pH 6.8, B: 0.1% formic acid in acetonitrile): 0 min (90% A), 1 min (90% A), 3 min (10% A), 4 min (10% A), 4.50 min (90% A), 6 min (90% A). The temperature of the Turbo V ion source is 500° C. (Example 2), 400° C. (Example 3) or 450° C. (Example 6). The following MS instrument parameters are used: curtain gas 15 units (Examples 2 and 3) or 10 units (Example 6), ion spray voltage 4.8 kV, gas 1 45 units (Example 2) or 50 units (Examples 3 and 6), gas 2 35 units (Examples 2 and 6) or 40 units (Example 3), CAD gas 4 units (Examples 2 and 6) or 8 units (Example 3). The substances are quantified by peak heights or areas using extracted ion chromatograms of specific MRM experiments.

The plasma concentration/time plots determined are used to calculate the pharmacokinetic parameters such as AUC, Cₘₐₓ, t112 (terminal half life), MRT (mean residence time) and CL (clearance), using the validated pharmacokinetic calculation program KinEx (Vers. 3).

Since the substance quantification is performed in plasma, it is necessary to determine the blood/plasma distribution of the substance in order to be able to adjust the pharmacokinetic parameters correspondingly. For this purpose, a defined amount of substance is incubated in heparinized whole blood of the species in question in a rocking roller mixer for 20 min. After centrifugation at 1000 g, the plasma concentration is measured (see above) and determined by calculating the quotient of the C_{blood}/Cₚₗₐₛₘₐ values.

Table 3 shows data of representative compounds of the present invention following intravenous administration of 0.3 mg/kg in rats:

TABLE 3

| Example | 2 | 3 | 6 |
|---|---|---|---|
| AUCₙₒᵣₘ [kg · h/l] | 13 | 0.93 | 3.5 |
| CL_{blood} [l/h/kg] | 0.13 | 0.93 | 0.37 |
| MRT [h] | 7.9 | 1.3 | 5.6 |
| t_{1/2} [h] | 6.1 | 1.0 | 4.4 |

### B-5. Metabolic Study

To determine the metabolic profile of the compounds according to the invention, they are incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds according to the invention were incubated with a concentration of about 0.1-10 μM. To this end, stock solutions of the compounds according to the invention having a concentration of 0.01-1 mM in acetonitrile were prepared, and then pipetted with 1:100 dilution into the incubation mixture. Liver microsomes and recombinant enzymes were incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation reactions were stopped with acetonitrile (final concentration about 30%) and the protein was centrifuged off at about 15 000×g. The samples thus stopped were either analyzed directly or stored at −20° C. until analysis.

The analysis is effected by means of high-performance liquid chromatography with ultraviolet and mass spectrometry detection (HPLC-UV-MS/MS). To this end, the supernatants of the incubation samples are chromatographed with suitable C18 reversed-phase columns and variable mobile phase mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic assessment of the compound according to the invention in the incubation mixtures.

### C. WORKING EXAMPLES OF PHARMACEUTICAL COMPOSITIONS

The compounds according to the invention can be converted to pharmaceutical formulations as follows:

### Tablet:

### Composition:

100 mg of the compound according to the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg, diameter 8 mm, radius of curvature 12 mm

### Production:

The mixture of compound according to the invention, lactose and starch is granulated with a 5% solution (w/w) of the PVP in water. The granules are dried and mixed with the magnesium stearate for 5 minutes. This mixture is pressed with a conventional tableting press (for tablet dimensions see above). The guide value used for the pressing is a pressing force of 15 kN.

Suspension which can be Administered Orally:

### Composition:

1000 mg of the inventive compound, 1000 mg of ethanol (96%), 400 mg of Rhodigel® (xanthan gum from FMC, Pennsylvania, USA) and 99 g of water.

A single dose of 100 mg of the compound according to the invention corresponds to 10 ml of oral suspension.

### Production:

The Rhodigel is suspended in ethanol and the compound according to the invention is added to the suspension. The water is added while stirring. The mixture is stirred for about 6 h until swelling of the Rhodigel is complete.

### Solution for Oral Administration:

### Composition:

500 mg of the compound according to the invention, 2.5 g of polysorbate and 97 g of polyethylene glycol 400. A single dose of 100 mg of the compound according to the invention corresponds to 20 g of oral solution.

### Production:

The compound according to the invention is suspended in the mixture of polyethylene glycol and polysorbate while stirring. The stirring operation is continued until dissolution of the inventive compound is complete.

i.v. solution:

The compound according to the invention is dissolved in a concentration below the saturation solubility in a physiologically acceptable solvent (e.g. isotonic saline, glucose solution 5% and/or PEG 400 solution 30%). The solution is subjected to sterile filtration and dispensed into sterile and pyrogen-free injection vessels.

1. A compound of formula (I) 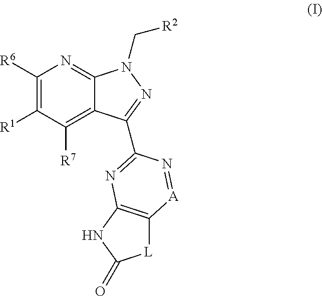
wherein
A represents nitrogen or CR³,
where
R³ represents hydrogen, deuterium, halogen, difluoromethyl, trifluoromethyl, (C₁-C₄)-alkyl, (C₂-C₄)-alkenyl, (C₂-C₄)-alkynyl, cyclopropyl, cyclobutyl, hydroxy, amino, phenyl or 5- or 6-membered heteroaryl,
in which (C₁-C₄)-alkyl, (C₂-C₄)-alkenyl, (C₂-C₄)-alkynyl, phenyl and 5- or 6-membered heteroaryl may be substituted by 1 to 3 substituents independently of one another selected from the group consisting of fluorine, difluoromethyl, trifluoromethyl, (C₁-C₄)-alkyl, difluoromethoxy, trifluoromethoxy, (C₁-C₄)-alkoxy, (C₁-C₄)-alkoxycarbonyl, cyclopropyl and cyclobutyl,
L represents a *—CR^{4A}R^{4B}—(CR^{5A}R^{5B})ₚ—# group,
where
* represents the point of attachment to the carbonyl group,
# represents the point of attachment to the pyrimidine ring or triazine ring,
p represents a number 0, 1 or 2,
R^{4A} represents hydrogen, fluorine, (C₁-C₄)-alkyl, hydroxy or amino,
in which (C₁-C₄)-alkyl may be substituted by 1 to 3 substituents independently of one another selected from the group consisting of fluorine, trifluoromethyl, hydroxy, hydroxycarbonyl, (C₁-C₄)-alkoxycarbonyl and amino,
R^{4B} represents hydrogen, fluorine, difluoromethyl, trifluoromethyl, (C₁-C₆)-alkyl, (C₁-C₄)-alkoxycarbonylamino, cyano, (C₃-C₇)-cycloalkyl, difluoromethoxy, trifluoromethoxy, phenyl or a group of the formula -M-R⁸,
in which (C₁-C₆)-alkyl may be substituted by 1 to 3 substituents independently of one another selected from the group consisting of fluorine, cyano, trifluoromethyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl and amino,
and in which
M represents a bond or (C₁-C₄)-alkanediyl,
R⁸ represents —(C═O)ₙ—OR⁹, —(C═O)ᵣ—NR⁹R¹⁰, —C(═S)—NR⁹R¹⁰, —NR⁹—(C═O)—R¹², —NR⁹—(C═O)—NR¹⁰R¹¹, —NR⁹—SO₂—NR¹⁰R¹¹, —NR⁹—SO₂—R¹², —S(O)ₛ—R¹², —SO₂—NR⁹R¹⁰, 4- to 7-membered heterocyclyl, phenyl or 5- or 6-membered heteroaryl,
in which
r represents the number 0 or 1,
s represents the number 0, 1 or 2,
R⁹, R¹⁰ and R¹¹ independently of one another each represent hydrogen, (C₁-C₆)-alkyl, (C₃-C₈)-cycloalkyl, 4- to 7-membered heterocyclyl, phenyl or 5- or 6-membered heteroaryl,
or
R⁹ and R¹⁰ together with the atom(s) to which they are respectively attached form a 4- to 7-membered heterocycle,
in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkylamino and di-(C₁-C₆)-alkylamino,
or
R¹⁰ and R¹¹ together with the atom(s) to which they are respectively attached form a 4- to 7-membered heterocycle,
in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkylamino and di-(C₁-C₆)-alkylamino,
R¹² represents (C₁-C₆)-alkyl or (C₃-C₇)-cycloalkyl,
or
R⁹ and R¹² together with the atom(s) to which they are respectively attached form a 4- to 7-membered heterocycle,
in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkylamino and di-(C₁-C₆)-alkylamino,
and
in which 4- to 7-membered heterocyclyl, phenyl and 5- or 6-membered heteroaryl for their part may be substituted by 1 to 3 substituents independently of one another selected from the group consisting of halogen, cyano, difluoromethyl, trifluoromethyl, (C₁-C₆)-alkyl, (C₃-C₇)-cycloalkyl, hydroxy, oxo, thioxo and (C₁-C₄)-alkoxy,
and
in which the aforementioned (C₁-C₄)-alkyl, (C₁-C₆)-alkyl, (C₃-C₈)-cycloalkyl and 4- to 7-membered heterocyclyl groups, unless stated otherwise, may each independently of one another additionally be substituted by 1 to 3 substituents independently of one another selected from the group consisting of fluorine, difluoromethyl, trifluoromethyl, (C₁-C₆)-alkyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl, amino, phenyl, 4- to 7-membered heterocyclyl and 5- or 6-membered heteroaryl,
or
R^{4A} and R^{4B} together with the carbon atom to which they are attached form a (C₂-C₄)-alkenyl group, an oxo group, a 3- to 6-membered carbocycle or a 4- to 7-membered heterocycle,
in which the 3- to 6-membered carbocycle and the 4- to 7-membered heterocycle may be substituted by 1 or 2 substituents independently of one another selected from the group consisting of fluorine and (C₁-C₄)-alkyl,
R^{5A} represents hydrogen, fluorine, (C₁-C₄)-alkyl, (C₁-C₄)-alkoxycarbonyl or hydroxy,
R^{5B} represents hydrogen, fluorine, (C₁-C₄)-alkyl or trifluoromethyl,
R¹ represents hydrogen, halogen, cyano, difluoromethyl, trifluoromethyl, (C₁-C₄)-alkyl or (C₃-C₇)-cycloalkyl,
R² represents 5- or 6-membered heteroaryl,
where 5- and 6-membered heteroaryl may be substituted by 1 or 2 fluorine substituents,
R⁶ represents hydrogen, cyano, difluoromethyl, trifluoromethyl, (C₁-C₄)-alkyl or (C₃-C₇)-cycloalkyl,
R⁷ represents hydrogen, cyano, difluoromethyl, trifluoromethyl, (C₁-C₄)-alkyl or (C₃-C₇)-cycloalkyl,
or an N-oxide, salt, or a salt of the N-oxide thereof.

2. The compound of claim 1, wherein
A represents nitrogen or CR³,
where
R³ represents hydrogen, deuterium, fluorine, iodine, difluoromethyl, trifluoromethyl, (C₁-C₄)-alkyl, vinyl, allyl, ethynyl, cyclopropyl, cyclobutyl, hydroxy, pyrazolyl or pyridyl,
where (C₁-C₄)-alkyl, vinyl, allyl, ethynyl and pyridyl may be substituted by 1 or 2 substituents independently of one another selected from the group consisting of methyl, cyclopropyl and cyclobutyl,
L represents a *—CR^{4A}R^{4B}—(CR^{5A}R^{5B})ₚ—# group,
where
* represents the point of attachment to the carbonyl group,
# represents the point of attachment to the pyrimidine ring or triazine ring,
p represents a number 0 or 1,
R^{4A} represents hydrogen, fluorine, methyl, ethyl, hydroxy or amino,
R^{4B} represents hydrogen, fluorine, difluoromethyl, trifluoromethyl, (C1-C4)-alkyl, methoxycarbonylamino, cyano, cyclopropyl, cyclobutyl, cyclopentyl, phenyl or a group of the formula -M-R⁸,
in which (C₁-C₄)-alkyl may be substituted by 1 to 3 substituents independently of one another selected from the group consisting of fluorine, cyano, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
and in which
M represents a bond or methylene,
R⁸ represents —(C═O)ₙ—NR⁹R¹⁰, —C(═S)—NR⁹R¹⁰, oxadiazolonyl, oxadiazolethionyl, phenyl, oxazolyl, thiazolyl, pyrazolyl, triazolyl, oxadiazolyl, thiadiazolyl, pyridyl, pyrimidinyl or pyrazinyl,
in which
r represents the number 0 or 1,
R⁹ and R¹⁰ independently of one another each represent hydrogen, methyl, ethyl, isopropyl, cyclopropyl, cyclobutyl, cyclopentyl, oxetanyl, azetidinyl, tetrahydrofuranyl, pyrrolidinyl, tetrahydropyranyl, piperidinyl, piperazinyl, morpholinyl, phenyl, pyrazolyl or pyridyl,
in which methyl, ethyl and isopropyl may additionally be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
and
in which oxadiazolonyl, oxadiazolethionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
or
R^{4A} and R^{4B} together with the carbon atom to which they are attached form a cyclopropyl, cyclobutyl, cyclopentyl, azetidinyl, tetrahydrofuranyl, pyrrolidinyl or tetrahydropyranyl ring,
in which the cyclopropyl, cyclobutyl, cyclopentyl, azetidinyl, tetrahydrofuranyl, pyrrolidinyl and tetrahydropyranyl ring may be substituted by 1 or 2 substituents independently of one another selected from the group consisting of fluorine and methyl,
R^{5A} represents hydrogen, fluorine, methyl, ethyl or hydroxy,
R^{5B} represents hydrogen, fluorine, methyl, ethyl or trifluoromethyl,
R¹ represents hydrogen or fluorine,
R² represents thienyl, pyridyl, pyrimidinyl, pyrazinyl or pyridazinyl,
where thienyl, pyridyl, pyrimidinyl, pyrazinyl and pyridazinyl may be substituted by 1 or 2 fluorine substituents,
R⁶ represents hydrogen or methyl,
R⁷ represents hydrogen,
or a salt thereof.

3. The compound of claim 1, wherein
A represents nitrogen or CR³,
where
R³ is hydrogen, fluorine, difluoromethyl, trifluoromethyl, methyl, ethyl, cyclopropyl or cyclobutyl,
L represents a *—CR^{4A}R^{4B}—(CR^{5A}R^{5B})ₚ—# group,
where
* represents the point of attachment to the carbonyl group,
# represents the point of attachment to the pyrimidine ring or triazine ring,
p represents a number 0,
R^{4A} represents hydrogen, fluorine, methyl, ethyl, hydroxy or amino,
R^{4B} represents hydrogen, fluorine, difluoromethyl, trifluoromethyl, methyl, ethyl, methoxycarbonylamino, cyclopropyl, cyclobutyl, cyclopentyl or a group of the formula -M-R⁸,
in which methyl and ethyl may be substituted by 1 to 3 substituents independently of one another selected from the group consisting of fluorine, cyano, trifluoromethyl, cyclopropyl, cyclobutyl, hydroxy, difluoromethoxy, trifluoromethoxy, methoxy, ethoxy, hydroxycarbonyl, methoxycarbonyl, ethoxycarbonyl and amino,
and in which
M represents a bond,
R⁸ represents —(C═O)ᵣ—NR⁹R¹⁰, phenyl, thiazolyl, triazolyl, oxadiazolyl, thiadiazolyl or pyrimidinyl,
in which
r represents the number 1,
R⁹ and R¹⁰ independently of one another each represent hydrogen or cyclopropyl,
and
in which phenyl, thiazolyl, triazolyl, oxadiazolyl, thiadiazolyl and pyrimidinyl for their part may be substituted by 1 or 2 substituents independently of one another selected from the group consisting of fluorine, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl and cyclobutylmethyl,
or
R^{4A} and R^{4B} together with the carbon atom to which they are attached form a cyclopropyl, cyclobutyl, cyclopentyl, azetidinyl, tetrahydrofuranyl, pyrrolidinyl or tetrahydropyranyl ring,
in which the cyclopropyl, cyclobutyl, cyclopentyl, azetidinyl, tetrahydrofuranyl, pyrrolidinyl and tetrahydropyranyl ring may be substituted by 1 or 2 substituents independently of one another selected from the group consisting of fluorine and methyl,
R¹ represents hydrogen or fluorine,
R² represents thienyl, pyridyl or pyrimidinyl,
where thienyl, pyridyl and pyrimidinyl may be substituted by 1 or 2 fluorine substituents,
R⁶ represents hydrogen or methyl,
R⁷ represents hydrogen,
or a salt thereof.

4. The compound of claim 1, wherein
A represents nitrogen or CR³,
where
R³ represents hydrogen,
L represents a *—CR^{4A}R^{4B}—(CR^{5A}R_{5B})ₚ—# group,
where
* represents the point of attachment to the carbonyl group,
# represents the point of attachment to the pyrimidine ring or triazine ring,
represents a number 0,
R^{4A} represents hydrogen, fluorine, methyl or hydroxy,
R^{4B} represents hydrogen, fluorine, trifluoromethyl, 2,2,2-trifluoroethyl or methyl,
R¹ represents hydrogen or fluorine,
R² represents thienyl, pyridyl or pyrimidinyl,
where thienyl, pyridyl and pyrimidinyl may be substituted by 1 or 2 fluorine substituents,
R⁶ represents hydrogen,
R⁷ represents hydrogen,
or salt thereof.

5. A process for preparing a compound of formula (I) as defined in claim 1, comprising reacting the compound of formula (II) 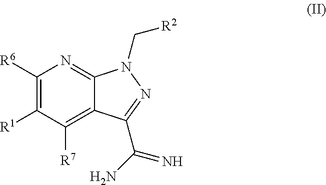
in which R¹, R², R⁶ and R⁷ each have the meanings given in claim 1,
[A] in an inert solvent in the presence of a suitable base with a compound of formula (III) 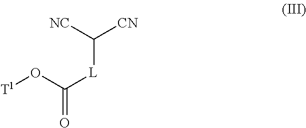
in which L has the meaning given in claim 1 and
T¹ represents (C₁-C₄)-alkyl
to give a compound of formula (IV) 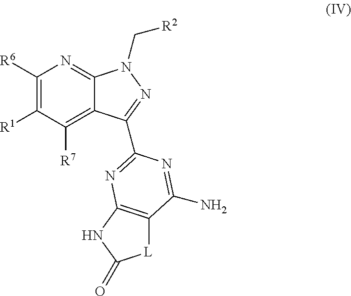
in which L, R¹, R², R⁶ and R⁷ each have the meanings given in claim 1,
converting the compound of formula (IV) with isopentyl nitrite and a halogen equivalent into a compound of formula (V) 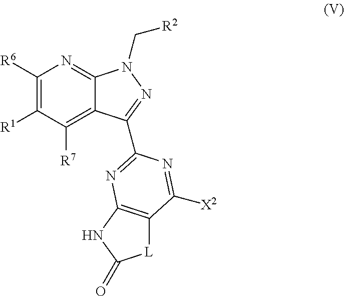
in which L, R¹, R², R⁶ and R⁷ each have the meanings given in claim 1 and
X² represents bromine or iodine,
and reacting the compound of formula (V) in an inert solvent, in the presence of a suitable transition metal catalyst, to give a compound of formula (I-A) 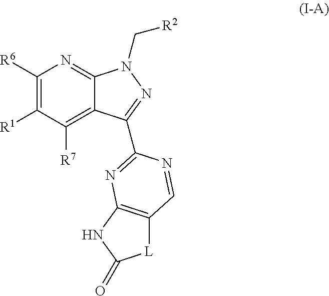
in which L, R¹, R², R⁶ and R⁷ each have the meanings given in claim 1,
or
[B] in an inert solvent in the presence of a suitable base with hydrazine hydrate to give a compound of formula (VI) 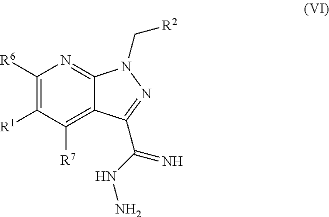
in which R¹, R², R⁶ and R⁷ each have the meanings given in claim 1,
reacting the compound of formula (VI) with a compound of formula (VII) 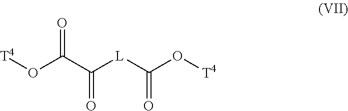
in which L has the meaning given in claim 1 and
T⁴ represents (C₁-C₄)-alkyl
to give a compound of formula (VIII) 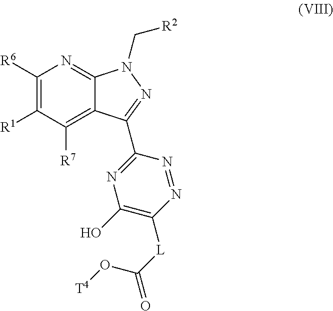
in which L, R¹, R², R⁶, and R⁷ each have the meanings given in claim 1 and T⁴ has the meaning given above,
converting the compound of formula (VIII) with phosphoryl chloride into a compound of formula (IX) 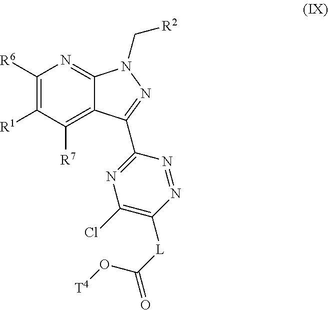
in which L, R¹, R², R⁶, and R⁷ each have the meanings given in claim 1 and T⁴ has the meaning given above,
and reacting the compound of formula (IX) directly with ammonia to give a compound of formula (X) 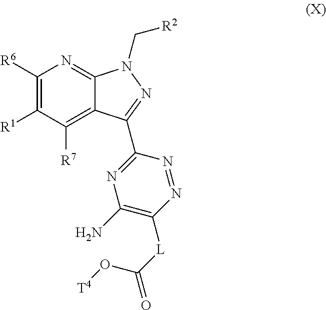
in which L, R¹, R², R⁶, and R⁷ each have the meanings given in claim 1 and T⁴ has the meaning given above,
and cyclizing the compound of formula (X) in an inert solvent, optionally in the presence of a suitable base, to give a compound of the formula (I-B) 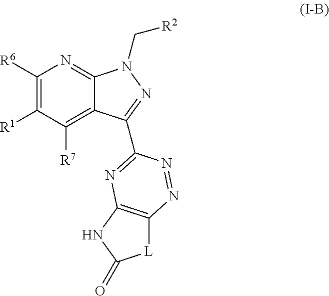
in which L, R¹, R², R⁶ and R⁷ each have the meanings given in claim 1 to,
and optionally converting the resulting compounds of formulae (I-A) and (I B) with the appropriate (i) solvents and/or (ii) acids or base into a salt thereof.

6. (canceled)

7. (canceled)

8. (canceled)

9. A pharmaceutical composition comprising a compound claim 1 and an inert, non-toxic, pharmaceutically suitable excipient

10. The pharmaceutical composition of claim 9, further comprising an active ingredient selected from the group consisting of an organic nitrates, an NO donors, a cGMP-PDE inhibitors, an agents having antithrombotic activity, an agents lowering blood pressure, and an agents altering lipid metabolism.

11. (canceled)

12. A method for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis comprising administering an effective amount of at least one compound of claim 1 to a human or animal in need thereof.

13. A method for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis comprising administering an effective amount of the pharmaceutical composition of claim 9 to a human or animal in need thereof.
